(19) United States
(12) Patent Application Publication
(10) Pub. No.: US 2025/0059585 A1
(43) Pub. Date: Feb. 20, 2025

(54) METHODS OF REMOVING EMBEDDING AGENTS FROM EMBEDDED SAMPLES

(71) Applicant: Foundation Medicine, Inc., Boston, MA (US)

(72) Inventors: Christine Marie MALBOEUF, Sharon, MA (US); Daniela Beatriz MUNAFO, South Hamilton, MA (US)

(73) Assignee: Foundation Medicine, Inc., Boston, MA (US)

(21) Appl. No.: 18/720,536

(22) PCT Filed: Dec. 15, 2022
(86) PCT No.: PCT/US2022/081719

§ 371 Date: Jun. 14, 2024

Related U.S. Application Data
(60) Provisional application No. 63/290,537, filed on Dec. 16, 2021.

(51) Int. Cl. C12Q 1/6806; C12N 15/10; C40B 50/00

(52) U.S. Cl. CPC C12Q 1/6806; C12N 15/1003; C40B 50/00

(57) ABSTRACT

The present disclosure provides methods of detecting analytes such as RNA and/or DNA, extracting analytes such as RNA and/or DNA, improving library construction for nucleic acid sequencing, and reducing the level of embedding agent in analyte samples such as RNA and/or DNA samples that are extracted from embedded samples such as paraffin-embedded samples.

### CROSS-REFERENCE TO RELATED APPLICATIONS

This application claims priority from U.S. Provisional Patent Application No. 63/290,537, filed Dec. 16, 2021, the contents of which are herein incorporated by reference in their entirety.

### FIELD

The present disclosure relates to methods of removing embedding agents such as paraffin from embedded samples. The methods described herein may be used to detect analytes such as RNA and/or DNA, extract analytes such as RNA and/or DNA, and improve library construction for nucleic acid sequencing from embedded samples, as well as methods of diagnosis, assessment, and treatment of diseases such as cancer.

### BACKGROUND

Cancer represents the phenotypic end-point of multiple genetic lesions that endow cells with a full range of biological properties required for tumorigenesis. Indeed, a hallmark genomic feature of many cancers is the presence of numerous complex chromosome structural aberrations, including translocations, intra-chromosomal inversions, point mutations, deletions, gene copy number changes, gene expression level changes, gene fusions, and germline mutations, among others. The presence of these hallmark genomic features can serve as biomarkers for cancer.

One method of detecting such biomarkers is the analysis of nucleic acids extracted from tumor cells in tissue samples, such as formalin-fixed paraffin-embedded (FFPE) tissues. In particular, it is desirable to extract both RNA and DNA from a single tissue sample. Methods of extracting RNA and/or DNA from paraffin-embedded tissue samples involve a step of removing the paraffin prior to nucleic acid extraction. One method of removing paraffin from embedded tissues involves dissolving the paraffin in a toxic organic solvent such as xylene, or another miscible solvent. Such solvent-based methods of removing paraffin are not amenable to robotic automation, for example, for the purpose of multiplexing nucleic acid sample preparation. Accordingly, there exists a need in the art for methods of removing embedding agents such as paraffin from embedded samples, particularly, methods that are amenable to robotic automation and high-throughput sample preparation.

All references cited herein, including patents, patent applications and publications, are hereby incorporated by reference in their entirety. To the extent that any reference incorporated by reference conflicts with the instant disclosure, the instant disclosure shall control.

### BRIEF SUMMARY

Provided herein is a method of detecting alterations in RNA and/or DNA, wherein the method comprises: a) providing a paraffin-embedded sample; b) removing paraffin from the paraffin-embedded sample, thereby generating a deparaffinized sample, wherein the paraffin is removed by inducing a phase transition in the paraffin and separating the paraffin from the sample; c) extracting RNA and/or DNA from the deparaffinized sample; and d) analyzing the RNA and/or DNA to detect alterations in the RNA and/or DNA. In some embodiments, the detection of alterations in the RNA and/or DNA is improved relative to a method wherein the paraffin is dissolved with a miscible solvent, optionally wherein the miscible solvent is xylene, ethyl acetate, CitriSolv™, or UltraClear™. In some embodiments, the detection of alterations in the RNA and/or DNA is improved relative to a method wherein the paraffin is removed by cutting the paraffin from the paraffin-embedded sample. In some embodiments, the detection of alterations in the RNA and/or DNA is improved relative to a method wherein the paraffin-embedded sample is not deparaffinized. In some embodiments, the alteration in the RNA and/or DNA is selected from the group consisting of: a) a copy number alteration; b) a point mutation; c) an in-frame deletion of one or more codons; d) an intragenic deletion; e) an intragenic insertion; f) a deletion of a full gene; g) an inversion; h) an interchromosomal translocation; i) a tandem duplication; j) a gene fusion; k) a genomic rearrangement that comprises an intron sequence; and/or l) a gene amplification or duplication. In some embodiments, the alteration in the RNA and/or the DNA is a copy number alteration.

Further provided herein is a method of extracting RNA and/or DNA, wherein the method comprises: a) providing a paraffin-embedded sample; b) removing paraffin from the paraffin-embedded sample, thereby generating a deparaffinized sample, wherein the paraffin is removed by inducing a phase transition in the paraffin and separating the paraffin from the sample; and c) extracting RNA and/or DNA from the deparaffinized sample. In some embodiments, the extraction of the RNA and/or DNA is improved relative to a method wherein the paraffin is dissolved with a miscible solvent, optionally wherein the miscible solvent is xylene, ethyl acetate, CitriSolv™, or UltraClear™. In some embodiments, the extraction of the RNA and/or DNA is improved relative to a method wherein the paraffin is removed by cutting the paraffin from the paraffin-embedded sample.

Further provided herein is a method of improving library construction for nucleic acid sequencing, wherein the method comprises: a) providing a paraffin-embedded sample; b) removing paraffin from the paraffin-embedded sample, thereby generating a deparaffinized sample, wherein the paraffin is removed by inducing a phase transition in the paraffin and separating the paraffin from the sample; c) extracting RNA and/or DNA from the deparaffinized sample; and d) preparing a sequencing library for sequencing the RNA and/or the DNA. In some embodiments, the preparation of the sequencing library is improved relative to a method wherein the paraffin is dissolved with a miscible solvent, optionally wherein the miscible solvent is xylene, ethyl acetate, CitriSolv™, or UltraClear™. In some embodiments, the preparation of the sequencing library is improved relative to a method wherein the paraffin is removed by cutting the paraffin from the paraffin-embedded sample.

Further provided herein is a method of reducing the level of paraffin in an RNA or DNA sample extracted from a paraffin-embedded sample, wherein the method comprises: a) providing a paraffin-embedded sample; b) removing paraffin from the paraffin-embedded sample, thereby generating a deparaffinized sample, wherein the paraffin is removed by inducing a phase transition in the paraffin and separating the paraffin from the sample; c) extracting RNA and/or DNA from the deparaffinized sample; and d) purifying the extracted RNA and/or DNA to provide an RNA and/or DNA sample. In some embodiments, the level of paraffin in the RNA and/or DNA sample is reduced relative to a method wherein the paraffin is dissolved with a miscible solvent, optionally wherein the miscible solvent is xylene, ethyl acetate, CitriSolv™, or UltraClear™. In some embodiments, the level of paraffin in the RNA and/or DNA sample is reduced relative to a method wherein the paraffin is removed by cutting the paraffin from the paraffin-embedded sample.

Further provided herein is a method of improving separation of paraffin from a paraffin-embedded sample, wherein the method comprises: a) providing a paraffin-embedded sample; b) removing paraffin from the paraffin-embedded sample, thereby generating a deparaffinized sample, wherein the paraffin is removed by inducing a phase transition in the paraffin and separating the paraffin from the sample; and c) extracting RNA and/or DNA from the deparaffinized sample. In some embodiments, the separation of paraffin from the paraffin-embedded sample is improved relative to a method wherein the paraffin is dissolved with a miscible solvent, optionally wherein the miscible solvent is xylene, ethyl acetate, CitriSolv™, or UltraClear™. In some embodiments, the separation of paraffin from the paraffin-embedded sample is improved relative to a method wherein the paraffin is removed by cutting the paraffin from the paraffin-embedded sample. In some embodiments, the method is performed in a liquid handling robot comprising a filter, wherein the improved separation of paraffin results in less clogging of the filter relative to a method wherein the paraffin is dissolved with a miscible solvent, optionally wherein the miscible solvent is xylene, ethyl acetate, CitriSolv™, or UltraClear™. In some embodiments, the method is performed in a liquid handling robot comprising a filter, wherein the improved separation of paraffin results in less clogging of the filter relative to a method wherein the paraffin is removed by cutting the paraffin from the paraffin-embedded sample. In some embodiments, the filter is a filter in a spin column.

In some embodiments of the preceding methods, step b) does not comprise dissolving the paraffin with a miscible solvent, optionally wherein the miscible solvent is xylene, ethyl acetate, CitriSolv™, or UltraClear™. In some embodiments of the preceding methods, the phase transition is melting. In some embodiments of the preceding methods, step b) comprises heating the paraffin-embedded sample. In some embodiments of the preceding methods, the paraffin-embedded sample is heated to about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., or about 75° C. In some embodiments of the preceding methods, the paraffin-embedded sample is heated for about 5 minutes, about 10 minutes, about 15 minutes, about 20 minutes, about 25 minutes, about 30 minutes, or about 35 minutes. In some embodiments of the preceding methods, step b) comprises centrifuging and filtering the paraffin-embedded sample. In some embodiments of the preceding methods, the paraffin-embedded sample is centrifuged at about 1,700 rcf, about 1,750 rcf, about 1,800 rcf, about 1,850 rcf, or about 1,900 rcf. In some embodiments of the preceding methods, the paraffin-embedded sample is centrifuged at 1,811 rcf or greater. In some embodiments of the preceding methods, the paraffin-embedded sample is centrifuged at 1,811 rcf. In some embodiments of the preceding methods, step b) comprises heating and centrifuging the paraffin-embedded sample. In some embodiments of the preceding methods, the paraffin-embedded sample is heated to about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., or about 75° C. In some embodiments of the preceding methods, the paraffin-embedded sample is heated for about 5 minutes, about 10 minutes, about 15 minutes, about 20 minutes, about 25 minutes, about 30 minutes, or about 35 minutes. In some embodiments of the preceding methods, the paraffin-embedded sample is centrifuged at about 1,700 rcf, about 1,750 rcf, about 1,800 rcf, about 1,850 rcf, or about 1,900 rcf. In some embodiments of the preceding methods, the paraffin-embedded sample is centrifuged at 1,811 rcf or greater. In some embodiments of the preceding methods, the paraffin-embedded sample is centrifuged at 1,811 rcf. In some embodiments of the preceding methods, step b) comprises heating, centrifuging, and filtering the paraffin-embedded sample.

Further provided herein is a method of detecting alterations in RNA and/or DNA, wherein the method comprises: a) providing a paraffin-embedded sample; b) removing paraffin from the paraffin-embedded sample, thereby generating a deparaffinized sample, wherein the paraffin is removed by contacting the paraffin-embedded sample with an immiscible solvent and separating the paraffin from the sample; c) extracting RNA and/or DNA from the deparaffinized sample; and d) analyzing the RNA and/or DNA to detect alterations in the RNA and/or DNA. In some embodiments, the detection of alterations in the RNA and/or DNA is improved relative to a method wherein the paraffin is dissolved with a miscible solvent, optionally wherein the miscible solvent is xylene, ethyl acetate, CitriSolv™ or UltraClear™. In some embodiments, the detection of alterations in the RNA and/or DNA is improved relative to a method wherein the paraffin is removed by cutting the paraffin from the paraffin-embedded sample. In some embodiments, the detection of alterations in the RNA and/or DNA is improved relative to a method wherein the paraffin-embedded sample is not deparaffinized. In some embodiments, the alteration in the RNA and/or DNA is selected from the group consisting of: a) a copy number alteration; b) a point mutation; c) an in-frame deletion of one or more codons; d) an intragenic deletion; e) an intragenic insertion; f) a deletion of a full gene; g) an inversion; h) an interchromosomal translocation; i) a tandem duplication; j) a gene fusion; k) a genomic rearrangement that comprises an intron sequence; and/or 1) a gene amplification or duplication. In some embodiments, the alteration in the RNA and/or the DNA is a copy number alteration.

Further provided herein is a method of extracting RNA and/or DNA, wherein the method comprises: a) providing a paraffin-embedded sample; b) removing paraffin from the paraffin-embedded sample, thereby generating a deparaffinized sample, wherein the paraffin is removed by contacting the paraffin-embedded sample with an immiscible solvent and separating the paraffin from the sample; and c) extracting RNA and/or DNA from the deparaffinized sample. In some embodiments, the extraction of the RNA and/or DNA is improved relative to a method wherein the paraffin is dissolved with a miscible solvent, optionally wherein the miscible solvent is xylene, ethyl acetate, CitriSolv™, or UltraClear™ In some embodiments, the extraction of the RNA and/or DNA is improved relative to a method wherein the paraffin is removed by cutting the paraffin from the paraffin-embedded sample.

Further provided herein is a method of improving library construction for nucleic acid sequencing, wherein the method comprises: a) providing a paraffin-embedded sample; b) removing paraffin from the paraffin-embedded sample, thereby generating a deparaffinized sample, wherein the paraffin is removed by contacting the paraffin-embedded sample with an immiscible solvent and separating the paraffin from the sample; c) extracting RNA and/or DNA from the deparaffinized sample; and d) preparing a sequencing library for sequencing the RNA and/or the DNA. In some embodiments, the preparation of the sequencing library is improved relative to a method wherein the paraffin is dissolved with a miscible solvent, optionally wherein the miscible solvent is xylene, ethyl acetate, CitriSolv™, or UltraClear™ In some embodiments, the preparation of the sequencing library is improved relative to a method wherein the paraffin is removed by cutting the paraffin from the paraffin-embedded sample.

Further provided herein is a method of reducing the level of paraffin in an RNA or DNA sample extracted from a paraffin-embedded sample, wherein the method comprises: a) providing a paraffin-embedded sample; b) removing paraffin from the paraffin-embedded sample, thereby generating a deparaffinized sample, wherein the paraffin is removed by contacting the paraffin-embedded sample with an immiscible solvent and separating the paraffin from the sample; c) extracting RNA and/or DNA from the deparaffinized sample; and d) purifying the extracted RNA and/or DNA to provide an RNA and/or DNA sample. In some embodiments, the level of paraffin in the RNA and/or DNA sample is reduced relative to a method wherein the paraffin is dissolved with a miscible solvent, optionally wherein the miscible solvent is xylene, ethyl acetate, CitriSolv™, or UltraClear™. In some embodiments, the level of paraffin in the RNA and/or DNA sample is reduced relative to a method wherein the paraffin is removed by cutting the paraffin from the paraffin-embedded sample.

Further provided herein is a method of improving separation of paraffin from a paraffin-embedded sample, wherein the method comprises: a) providing a paraffin-embedded sample; b) removing paraffin from the paraffin-embedded sample, thereby generating a deparaffinized sample, wherein the paraffin is removed by contacting the paraffin-embedded sample with an immiscible solvent and separating the paraffin from the sample; and c) extracting RNA and/or DNA from the deparaffinized sample. In some embodiments, the separation of paraffin from the paraffin-embedded sample is improved relative to a method wherein the paraffin is dissolved with a miscible solvent, optionally wherein the miscible solvent is xylene, ethyl acetate, CitriSolv™, or UltraClear™. In some embodiments, the separation of paraffin from the paraffin-embedded sample is improved relative to a method wherein the paraffin is removed by cutting the paraffin from the paraffin-embedded sample. In some embodiments, the method is performed in a liquid handling robot comprising a filter, wherein the improved separation of paraffin results in less clogging of the filter relative to a method wherein the paraffin is dissolved with a miscible solvent, optionally wherein the miscible solvent is xylene, ethyl acetate, CitriSolv™, or UltraClear™. In some embodiments, the method is performed in a liquid handling robot comprising a filter, wherein the improved separation of paraffin results in less clogging of the filter relative to a method wherein the paraffin is removed by cutting the paraffin from the paraffin-embedded sample. In some embodiments, the filter is a filter in a spin column.

In some embodiments of the preceding methods, step b) does not comprise dissolving the paraffin with a miscible solvent, optionally wherein the miscible solvent is xylene, ethyl acetate, CitriSolv™, or UltraClear™. In some embodiments of the preceding methods, the immiscible solvent is mineral oil. In some embodiments, the paraffin-embedded sample is contacted with about 300, 350, 400, 450, 500, or 550 μL of mineral oil. In some embodiments, the paraffin-embedded sample is contacted with mineral oil at a mineral oil to paraffin-embedded sample ratio of about 1:4, 1:2, 1:1, 2:1, 4:1, 5:1, 6:1, 7:1, 8:1, 9:1, 10:1, 15:1, 20:1, 30:1, 40:1, or 50:1. In some embodiments, step b) comprises incubating the paraffin-embedded sample in mineral oil at about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., or about 75° C. In some embodiments, the mineral oil contacts the paraffin-embedded sample for about 5 minutes, about 10 minutes, about 15 minutes, about 20 minutes, about 25 minutes, about 30 minutes, or about 35 minutes. In some embodiments, the mineral oil contacts the paraffin-embedded sample while shaking at about 25 rpm, about 50 rpm, about 100 rpm, about 200 rpm, about 300 rpm, about 400 rpm, about 500 rpm, about 600 rpm, about 700 rpm, about 800 rpm, about 900 rpm, about 1000 rpm, or about 1100 rpm.

In some embodiments of the preceding methods, step b) comprises contacting the sample with an immiscible solvent, centrifuging, and filtering the paraffin-embedded sample. In some embodiments, the paraffin-embedded sample is centrifuged at about 1,700 rcf, about 1,750 rcf, about 1,800 rcf, about 1,850 rcf, or about 1,900 rcf. In some embodiments, the paraffin-embedded sample is centrifuged at 1,811 rcf or greater. In some embodiments, the paraffin-embedded sample is centrifuged at 1,811 rcf.

In some embodiments of the preceding methods, the separation of the paraffin from the sample is automated. In some embodiments of the preceding methods, step b) is automated. In some embodiments of the preceding methods, the method is automated. In some embodiments of the preceding methods, two or more paraffin-embedded samples are processed in parallel. In some embodiments, 12, 24, 48, or 96 paraffin-embedded samples are processed in parallel. In some embodiments of the preceding methods, the method is performed using a liquid handling robot. In some embodiments, the liquid handling robot is an Agilent, BioTek, Hamilton, Tecan, or ThermoFisher Scientific liquid handling robot, optionally wherein the liquid handling robot is a Hamilton AutoLys STAR, a Hamilton STAR, a BioTek Dispenser, or a KingFisher Flex. In some embodiments, the liquid handling robot is a Hamilton AutoLys STAR. In some embodiments of the preceding methods, step c) comprises extracting RNA and DNA from the deparaffinized sample. In some embodiments of the preceding methods, step a) comprises: i) identifying a target region comprising tumor cells of interest in a paraffin-embedded tissue; ii) extracting the paraffin-embedded sample from the paraffin-embedded tissue; iii) identifying the location of the paraffin-embedded sample in the paraffin-embedded tissue; and iv) if the location of the paraffin-embedded sample overlaps with the target region comprising tumor cells of interest, extracting RNA and/or DNA from the sample. In some embodiments, the location of the paraffin-embedded sample does not overlap with the target region comprising tumor cells of interest, steps ii) and iii) are repeated. In some embodiments, step ii) comprises extracting the paraffin-embedded sample using a needle. In some embodiments, the needle is punched through the paraffin-embedded tissue, thereby extracting the paraffin-embedded sample. In some embodiments, the needle is a disposable needle. In some embodiments, the needle is a 13 gauge needle, 14 gauge needle, a 15 gauge needle, a 16 gauge needle, a 17 gauge needle, a 18 gauge needle, a 19 gauge needle, a 20 gauge needle, or a 21 gauge needle. In some embodiments, the paraffin-embedded sample extracted from the paraffin-embedded tissue is about 0.8, 0.9, 1.0, 1.2, 1.4, 1.6, 1.8, 2.0, or 2.3 mm in diameter. In some embodiments, step ii) comprises extracting the paraffin-embedded sample using laser microdissection (LMD) or a razor blade. In some embodiments, step iii) comprises preparing a slide of a section of the paraffin-embedded tissue. In some embodiments, the section of the paraffin-embedded tissue is stained. In some embodiments, the section of the paraffin-embedded tissue is Haematoxylin and Eosin (H&E) stained. In some embodiments, step iii) is performed by visual inspection. In some embodiments, step iii) is performed by a computer system. In some embodiments, step iii) is performed using an image analysis system.

In some embodiments of the preceding methods, the paraffin-embedded sample is from an individual known to have cancer or suspected of having cancer. In some embodiments of the preceding methods, the paraffin-embedded sample is from a biopsy, optionally a tumor biopsy. In some embodiments of the preceding methods, the paraffin-embedded sample is a fixed paraffin-embedded sample. In some embodiments, the fixed paraffin-embedded sample is selected from the group consisting of a formalin-fixed sample, an ethanol-fixed sample, and a methanol-fixed sample. In some embodiments of the preceding methods, the paraffin-embedded sample is derived from a formalin-fixed paraffin-embedded (FFPE) tissue. In some embodiments of the preceding methods, the paraffin-embedded sample is derived from a cryopreserved tissue. In some embodiments of the preceding methods, the paraffin-embedded sample is derived from a fresh-frozen tissue. In some embodiments, the fresh-frozen tissue is frozen in an optimal cutting temperature (OCT) compound. In some embodiments of the preceding methods, the RNA is extracted before the DNA is extracted. In some embodiments, the method further comprises digesting the paraffin-embedded sample before step b). In some embodiments, the paraffin-embedded sample is digested using a proteinase. In some embodiments, the proteinase is proteinase K. In some embodiments, the paraffin-embedded sample is digested using about 10 μL, about 15 μL, about 20 μL, about 25 μL, about 30 μL, or about 35 μL of 20 mg/mL proteinase K. In some embodiments, the paraffin-embedded sample is incubated with the proteinase at about 50° C., about 51° C., about 52° C., about 53° C., about 54° C., about 55° C., about 56° C., about 57° C., about 58° C., about 59° C., about 60° C., about 61° C., or about 62° C. In some embodiments, the paraffin-embedded sample is incubated with the proteinase for about 10 minutes, about 11 minutes, about 12 minutes, about 13 minutes, about 14 minutes, about 15 minutes, about 16 minutes, about 17 minutes, about 18 minutes, about 19 minutes, or about 20 minutes. In some embodiments, the paraffin-embedded sample is incubated with the proteinase shaking at about 500 rpm, 600 rpm, 700 rpm, 800 rpm, 900 rpm, 1000 rpm, 1100 rpm, 1200 rpm, 1300 rpm, 1400 rpm, or 1500 rpm. In some embodiments, the paraffin-embedded sample is partially digested or completely digested. In some embodiments, the method further comprises de-crosslinking the digested sample after step b). In some embodiments, de-crosslinking comprises heating the digested sample to 80-90° C. In some embodiments, the digested sample is heated for about 10 minutes, about 11 minutes, about 12 minutes, about 13 minutes, about 14 minutes, about 15 minutes, about 16 minutes, about 17 minutes, about 18 minutes, about 19 minutes, or about 20 minutes. In some embodiments, step c) comprises collecting a sample lysate comprising RNA from the digested paraffin-embedded sample, and purifying the RNA from the sample lysate comprising RNA. In some embodiments, the method further comprises completely digesting the digested paraffin-embedded sample. In some embodiments, the complete digestion is performed using a proteinase. In some embodiments, the proteinase is proteinase K. In some embodiments, the digested paraffin-embedded sample is digested using about 10 μL, about 15 μL, about 20 μL, about 25 μL, about 30 μL, or about 35 μL of 20 mg/mL proteinase K. In some embodiments, the digested paraffin-embedded sample is incubated with the proteinase at about 65° C., about 66° C., about 67° C., about 68° C., about 69° C., about 70° C., about 71° C., about 72° C., about 73° C., about 74° C., or about 75° C. In some embodiments, the digested paraffin-embedded sample is incubated with the proteinase for about 6 hours, about 7 hours, about 8 hours, about 9 hours, about 10 hours, about 11 hours, 12 hours, about 13 hours, about 14 hours, about 15 hours, about 16 hours, about 17 hours, about 18 hours, about 19 hours, about 20 hours, about 21 hours, or about 22 hours. In some embodiments, the digested paraffin-embedded sample is incubated with the proteinase shaking at about 500 rpm, 600 rpm, 700 rpm, 800 rpm, 900 rpm, 1000 rpm, 1100 rpm, 1200 rpm, 1300 rpm, 1400 rpm, or 1500 rpm. In some embodiments, the method further comprises collecting a sample lysate comprising DNA from the completely digested paraffin-embedded sample. In some embodiments, the method further comprises purifying the DNA from the sample lysate comprising DNA. In some embodiments, the DNA is extracted before the RNA is extracted. In some embodiments, the method further comprises completely digesting the paraffin-embedded sample after step b). In some embodiments, the complete digestion is performed using a proteinase. In some embodiments, the proteinase is proteinase K. In some embodiments, the paraffin-embedded sample is digested using about 10 μL, about 15 μL, about 20 μL, about 25 μL, about 30 μL, or about 35 L of 20 mg/mL proteinase K. In some embodiments, the paraffin-embedded sample is incubated with the proteinase at about 65° C., about 66° C., about 67° C., about 68° C., about 69° C., about 70° C., about 71° C., about 72° C., about 73° C., about 74° C., or about 75° C. In some embodiments, the paraffin-embedded sample is incubated with the proteinase for about 6 hours, about 7 hours, about 8 hours, about 9 hours, about 10 hours, about 11 hours, 12 hours, about 13 hours, about 14 hours, about 15 hours, about 16 hours, about 17 hours, about 18 hours, about 19 hours, about 20 hours, about 21 hours, or about 22 hours. In some embodiments, the method further comprises extracting the DNA from the completely digested paraffin-embedded sample. In some embodiments, the method further comprises extracting the RNA from the completely digested paraffin-embedded sample.

In some embodiments of the preceding methods, the method further comprises measuring the amount of RNA and/or DNA extracted from the paraffin-embedded sample. In some embodiments, the amount of RNA and/or DNA extracted from the paraffin-embedded sample is about 5 ng, about 10 ng, about 20 ng, about 30 ng, about 40 ng, about 50 ng, about 60 ng, about 70 ng, about 80 ng, about 100 ng, about 50 g, or about 50 mg. In some embodiments of the preceding methods, the method further comprises analyzing the RNA and/or the DNA extracted from the paraffin-embedded sample by one or more methods selected from the group consisting of a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), and mass-spectrometric genotyping. In some embodiments of the preceding methods, the method further comprises analyzing the RNA and/or the DNA extracted from the paraffin-embedded sample by next-generation sequencing. In some embodiments of the preceding methods, the method further comprises: e) optionally, ligating one or more adaptors onto the RNA and/or DNA extracted from the paraffin-embedded sample, thereby generating ligated nucleic acids; d) optionally, amplifying nucleic acids from the ligated nucleic acids; f) optionally, capturing a plurality of nucleic acids corresponding to one or more genes of interest; g) sequencing, by a sequencer, the plurality of nucleic acids to obtain a plurality of sequence reads corresponding to the one or more genes of interest; h) analyzing the plurality of sequence reads; and i) based on the analysis, detecting one or more mutations in a gene of interest. In some embodiments, the plurality of nucleic acids corresponding to the one or more genes of interest is captured from the amplified nucleic acids by hybridization with a bait molecule. In some embodiments, the plurality of nucleic acids corresponding to the one or more genes of interest is enriched from the amplified nucleic acids, optionally wherein the plurality of nucleic acids corresponding to the one or more genes of interest is enriched from the amplified nucleic acids by biotin/streptavidin tagging. In some embodiments, prior to step e) the RNA and/or DNA extracted from the paraffin-embedded sample are fragmented, optionally wherein the RNA and/or DNA extracted from the paraffin-embedded sample are fragmented by sonication. In some embodiments, the fragmented RNA and/or DNA extracted from the paraffin-embedded sample are end-repaired. In some embodiments, the end-repaired, fragmented RNA and/or DNA extracted from the sample are dA-tailed or dT-tailed.

In some embodiments of the preceding methods, the method further comprises analyzing the DNA extracted from the paraffin-embedded sample to detect a somatic mutation selected from the group consisting of: i) a point mutation; ii) an in-frame deletion of one or more codons; iii) an intragenic deletion; iv) an intragenic insertion; v) a deletion of a full gene; vi) an inversion; vii) an interchromosomal translocation; viii) a tandem duplication; ix) a gene fusion; x) a genomic rearrangement that comprises an intron sequence; and/or xi) a gene amplification or duplication. In some embodiments, the somatic mutation is in a gene, and wherein the gene is ABL1, AKT1, AKT2, AKT3, ALK, APC, AR, BRAF, CCND1, CDK4, CDKN2A, CEBPA, CTNNB1, EGFR, ERBB2, ESR1, FGFR1, FGFR2, FGFR3, FLT3, HRAS, JAK2, KIT, KRAS, MAP2K1, MAP2K2, MET, MLL, MYC, NF1, NOTCH1, NPM1, NRAS, NTRK3, PDGFRA, PIK3CA, PIK3CG, PIK3R1, PTCH1, PTCH2, PTEN, RB1, RET, SMO, STK11, SUFU, or TP53. In some embodiments, the somatic mutation is in a gene, and wherein the gene is ABL2, ARAF, ARFRP1, ARID1A, ATM, ATR, AURKA, AURKB, BAP1, BCL2, BCL2A1, BCL2L1, BCL2L2, BCL6, BRCA1, BRCA2, CBL, CARD11, CBL, CCND2, CCND3, CCNE1, CD79A, CD79B, CDH1, CDH2, CDH20, CDH5, CDK6, CDK8, CDKN2B, CDKN2C, CHEK1, CHEK2, CRKL, CRLF2, DNMT3A, DOT1L, EPHA3, EPHA5, EPHA6, EPHA7, EPHB1, EPHB4, EPHB6, ERBB3, ERBB4, ERG, ETV1, ETV4, ETV5, ETV6, EWSR1, EZH2, FANCA, FBXW7, FGFR4, FLT1, FLT4, FOXP4, GATA1, GNA11, GNAQ, GNAS, GPR124, GUCY1A2, HOXA3, HSP90AA1, IDH1, IDH2, IGF1R, IGF2R, IKBKE, IKZF1, INHBA, IRS2, JAK1, JAK3, JUN, KDM6A, KDR, LRP1B, LRP6, LTK, MAP2K4, MCL1, MDM2, MDM4, MEN1, MITF, MLH1, MPL, MRE11A, MSH2, MSH6, MTOR, MUTYH, MYCL1, MYCN, NF2, NKX2-1, NTRK1, NTRK2, PAK3, PAX5, PDGFRB, PKHD1, PLCG1, PRKDC, PTPN11, PTPRD, RAF1, RARA, RICTOR, RPTOR, RUNX1, SMAD2, SMAD3, SMAD4, SMARCA4, SMARCB1, SOX10, SOX2, SRC, TBX22, TET2, TGFBR2, TMPRSS2, TNFAIP3, TNK, TNKS2, TOP1, TSC1, TSC2, USP9X, VHL, or WT1.

In some embodiments of the preceding methods, the method further comprises analyzing the RNA extracted from the paraffin-embedded sample to detect an alternation selected from the group consisting of: i) gene fusions; ii) exon-skipping events; iii) splice variants; and/or iv) altered gene expression. In some embodiments of the preceding methods, the method further comprises detecting loss-of-heterozygosity (LOH) of one or more genes of interest in the paraffin-embedded sample. In some embodiments, the method further comprises detecting LOH of ST7/RAY1, ARH1/NOEY2, TSLC1, RB, PTEN, SMAD2, SMAD4, DCC, TP53, ATM, miR-15a, miR-16-1, NAT2, BRCA1, BRCA2, hOGG1, CDH1, IGF2, CDKN1C/P57, MEN1, PRKAR1A, H19, KRAS, BAP1, PTCH1, SMO, SUFU, NOTCH1, PPP6C, LATS1, CASP8, PTPN14, ARID1A, FBXW7, M6P/IGF2R, IFN-alpha, an olfactory receptor gene, CBFA2T3, DUTT1, FHIT, APC, P16, FCMD, TSC2, miR-34, c-MPL, RUNX3, DIRAS3, NRAS, miR-9, FAM50B, PLAGL1, ER, FLT3, ZDBF2, GPR1, c-KIT, NAP1L5, GRB10, EGFR, PEG10, BRAF, MEST, JAK2, DAPK1, LIT1, WT1, NF-1, PR, c-CBL, DLK1, AKT1, SNURF, a cytochrome P450 gene (CYP), ZNF587, SOCS1, TIMP2, RUNX1, AR, CEBPA, C19MC, EMP3, ZNF331, CDKN2A, PEG3, NNAT, GNAS, and/or GATA5 in the paraffin-embedded sample. In some embodiments, the method further comprises detecting LOH of a human leukocyte antigen (HLA) gene in the paraffin-embedded sample. In some embodiments, the method further comprises: ligating one or more adaptors onto one or more of the RNA and/or DNA extracted from the paraffin-embedded sample, thereby generating ligated nucleic acids; amplifying the ligated nucleic acids; capturing a plurality of nucleic acids corresponding to the HLA gene from the amplified nucleic acids using a bait molecule; sequencing the captured nucleic acids to obtain a plurality of sequence reads corresponding to the HLA gene; fitting, by one or more processors, one or more values associated with one or more of the plurality of sequence reads to a model; and based on the model, detecting LOH of the HLA gene and a relative binding propensity for an HLA allele of the HLA gene. In some embodiments, LOH of the HLA gene and relative binding propensity for an HLA allele of the HLA gene are detected by: a) obtaining an observed allele frequency for an HLA allele, wherein observed allele frequency corresponds to frequency of nucleic acid(s) encoding at least a portion of the HLA allele as detected among the plurality of sequence reads corresponding to the HLA gene; b) obtaining a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles; c) applying an objective function to measure a difference between the relative binding propensity and the observed allele frequency of the HLA allele; d) applying an optimization model to minimize the objective function; e) determining an adjusted allele frequency of the HLA allele based on the optimization model and the observed allele frequency; and f) determining that LOH has occurred when the adjusted allele frequency of the HLA allele is less than a predetermined threshold. In some embodiments, the paraffin-embedded sample is from an individual known to have cancer or suspected of having cancer, further comprising, based at least in part on detection of LOH of the HLA gene, administering an effective amount of a treatment other than an immune checkpoint inhibitor (ICI) to the individual. In some embodiments, the method further comprises, based at least in part on detection of LOH of the HLA gene, recommending a treatment other than an immune checkpoint inhibitor (ICI). In some embodiments, the method further comprises: detecting, or acquiring knowledge of, a high tumor mutational burden (TMB) in the paraffin-embedded sample. In some embodiments, the paraffin-embedded sample is from an individual known to have cancer or suspected of having cancer, further comprising, based at least in part on detection of LOH of the HLA gene and high TMB, administering an effective amount of an immune checkpoint inhibitor (ICI) to the individual. In some embodiments, the paraffin-embedded sample is from an individual known to have cancer or suspected of having cancer, further comprising, based at least in part on detection of LOH of the HLA gene and high TMB, recommending a treatment comprising an immune checkpoint inhibitor (ICI) to the individual. In some embodiments, the HLA gene is a human HLA-A, HLA-B, or HLA-C gene. In some embodiments, the TMB is determined based on a number of non-driver somatic coding mutations per megabase of genome sequenced.

In some embodiments of the preceding methods, the method further comprises detecting a loss-of-function mutation in a phosphatase and tensin homolog (PTEN) gene in the paraffin-embedded sample. In some embodiments, the loss-of-function mutation in a PTEN gene comprises one or more of an insertion, deletion or substitution of one or more nucleotides, a genomic rearrangement, an alteration in a promoter, a gene fusion, or a copy number alteration. In some embodiments, the loss-of-function mutation in a PTEN gene is detected in the paraffin-embedded sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping.

In some embodiments of the preceding methods, the method further comprises measuring the level of tumor mutational burden (TMB) in the paraffin-embedded sample. In some embodiments, a TMB of at least about 10 mutations/megabase (Mb) or at least about 20 mut/Mb is detected. In some embodiments, TMB is measured in the RNA and/or DNA from the paraffin-embedded sample by whole exome sequencing, whole genome sequencing, or gene-targeted sequencing. In some embodiments, TMB is measured on about 0.80 Mb of sequenced DNA. In some embodiments, TMB is measured on between about 0.83 Mb and about 1.14 Mb of sequenced DNA. In some embodiments, TMB is measured on about 1.1 Mb of sequenced DNA. In some embodiments, TMB is measured on up to about 1.1 Mb of sequenced DNA.

In some embodiments of the preceding methods, the method further comprises detecting homozygous single exon loss in the paraffin-embedded sample. In some embodiments, the homozygous single exon loss is detected in the RNA and/or DNA from the paraffin-embedded sample by whole exome sequencing, whole genome sequencing, or gene-targeted sequencing.

Further provided herein is a method of detecting an analyte, wherein the method comprises: a) providing an embedded sample comprising an analyte, wherein the sample is embedded in an embedding agent; b) removing the embedding agent from the embedded sample, thereby generating a de-embedded sample, wherein the embedding agent is removed by inducing a phase transition in the embedding agent and separating the embedding agent from the sample; c) extracting the analyte from the de-embedded sample; and d) analyzing the analyte to detect the analyte. In some embodiments, the detection the analyte is improved relative to a method wherein the embedding agent is dissolved with a miscible solvent, optionally wherein the miscible solvent is xylene, ethyl acetate, CitriSolv™, or UltraClear™ In some embodiments, the detection the analyte is improved relative to a method wherein the embedding agent is removed by cutting the embedding agent from the embedded sample. In some embodiments, the detection the analyte is improved relative to a method wherein the embedded sample is not de-embedded.

Further provided herein is a method of extracting an analyte, wherein the method comprises: a) providing an embedded sample comprising an analyte, wherein the sample is embedded in an embedding agent; b) removing the embedding agent from the embedded sample, thereby generating a de-embedded sample, wherein the embedding agent is removed by inducing a phase transition in the embedding agent and separating the embedding agent from the sample; and c) extracting the analyte from the de-embedded sample. In some embodiments, the extraction of the analyte is improved relative to a method wherein the embedding agent is dissolved with a miscible solvent, optionally wherein the miscible solvent is xylene, ethyl acetate, CitriSolv™, or UltraClear™. In some embodiments, the extraction of the analyte is improved relative to a method wherein the embedding agent is removed by cutting the embedding agent from the embedded sample.

Further provided herein is a method of reducing the level of embedding agent in an analyte sample extracted from an embedded sample, wherein the method comprises: a) providing an embedded sample comprising an analyte, wherein the sample is embedded in an embedding agent; b) removing the embedding agent from the embedded sample, thereby generating a de-embedded sample, wherein the embedding agent is removed by inducing a phase transition in the embedding agent and separating the embedding agent from the sample; c) extracting the analyte from the de-embedded sample; and d) purifying the extracted analyte to provide an analyte sample. In some embodiments, the level of embedding agent in the analyte sample is reduced relative to a method wherein the embedding agent is dissolved with a miscible solvent, optionally wherein the miscible solvent is xylene, ethyl acetate, CitriSolv™, or UltraClear™. In some embodiments, the level of embedding agent in the analyte sample is reduced relative to a method wherein the embedding agent is removed by cutting the embedding agent from the embedded sample.

Further provided herein is a method of improving separation of an embedding agent from an embedded sample, wherein the method comprises: a) providing an embedded sample comprising an analyte, wherein the sample is embedded in an embedding agent; b) removing the embedding agent from the embedded sample, thereby generating a de-embedded sample, wherein the embedding agent is removed by inducing a phase transition in the embedding agent and separating the embedding agent from the sample; and c) extracting an analyte from the de-embedded sample. In some embodiments, the separation of embedding agent from the embedded sample is improved relative to a method wherein the embedding agent is dissolved with a miscible solvent, optionally wherein the miscible solvent is xylene, ethyl acetate, CitriSolv™, or UltraClear™. In some embodiments, the separation of embedding agent from the embedded sample is improved relative to a method wherein the embedding agent is removed by cutting the embedding agent from the embedded sample. In some embodiments, the method is performed in a liquid handling robot comprising a filter, wherein the improved separation of embedding agent results in less clogging of the filter relative to a method wherein the embedding agent is dissolved with a miscible solvent, optionally wherein the miscible solvent is xylene, ethyl acetate, CitriSolv™, or UltraClear™. In some embodiments, the method is performed in a liquid handling robot comprising a filter, wherein the improved separation of embedding agent results in less clogging of the filter relative to a method wherein the embedding agent is removed by cutting the embedding agent from the embedded sample. In some embodiments, the filter is a filter in a spin column.

In some embodiments of the preceding methods, step b) does not comprise dissolving the embedding agent with a miscible solvent, optionally wherein the miscible solvent is xylene, ethyl acetate, CitriSolv™, or UltraClear™. In some embodiments of the preceding methods, the phase transition is selected from the group consisting of melting, freezing, vaporization, condensation, sublimation, and deposition. In some embodiments of the preceding methods, the phase transition is melting. In some embodiments of the preceding methods, step b) comprises heating, cooling, increasing the pressure, or decreasing the pressure of the embedded sample. In some embodiments of the preceding methods, step b) comprises heating the embedded sample. In some embodiments, the embedded sample is heated to about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., or about 75° C. In some embodiments, the embedded sample is heated for about 5 minutes, about 10 minutes, about 15 minutes, about 20 minutes, about 25 minutes, about 30 minutes, or about 35 minutes. In some embodiments of the preceding methods, step b) comprises centrifuging and filtering the embedded sample to separate the embedding from the sample. In some embodiments, the embedded sample is centrifuged at about 1,700 rcf, about 1,750 rcf, about 1,800 rcf, about 1,850 rcf, or about 1,900 rcf. In some embodiments, the embedded sample is centrifuged at 1,811 rcf or greater. In some embodiments, the embedded sample is centrifuged at 1,811 rcf. In some embodiments of the preceding methods, step b) comprises heating, centrifuging, and filtering the embedded sample to separate the embedding agent from the sample, thereby generating a de-embedded sample. In some embodiments of the preceding methods, step b) comprises heating and centrifuging the embedded sample to separate the embedding agent from the sample, thereby generating a de-embedded sample.

Further provided herein is a method of detecting an analyte, wherein the method comprises: a) providing an embedded sample comprising an analyte, wherein the sample is embedded in an embedding agent; b) removing the embedding agent from the embedded sample, thereby generating a de-embedded sample, wherein the embedding agent is removed by contacting the embedded sample with an immiscible solvent and separating the embedding agent from the sample; c) extracting the analyte from the de-embedded sample; and d) analyzing the analyte to detect the analyte. In some embodiments, the detection of the analyte is improved relative to a method wherein the embedding agent is dissolved with a miscible solvent, optionally wherein the miscible solvent is xylene, ethyl acetate, CitriSolv™, or UltraClear™. In some embodiments, the detection of the analyte is improved relative to a method wherein the embedding agent is removed by cutting the embedding agent from the embedded sample. In some embodiments, the detection the analyte is improved relative to a method wherein the embedded sample is not de-embedded.

Further provided herein is a method of extracting an analyte, wherein the method comprises: a) providing an embedded sample comprising an analyte, wherein the sample is embedded in an embedding agent; b) removing the embedding agent from the embedded sample, thereby generating a de-embedded sample, wherein the embedding agent is removed by contacting the embedded sample with an immiscible solvent and separating the embedding agent from the sample; and c) extracting the analyte from the de-embedded sample. In some embodiments, the extraction of the analyte is improved relative to a method wherein the embedding agent is dissolved with a miscible solvent, optionally wherein the miscible solvent is xylene, ethyl acetate, CitriSolv™, or UltraClear™. In some embodiments, the extraction of the analyte is improved relative to a method wherein the embedding agent is removed by cutting the embedding agent from the embedded sample.

Further provided herein is a method of reducing the level of embedding agent in an analyte sample extracted from an embedded sample, wherein the method comprises: a) providing an embedded sample comprising an analyte, wherein the sample is embedded in an embedding agent; b) removing the embedding agent from the embedded sample, thereby generating a de-embedded sample, wherein the embedding agent is removed by contacting the embedded sample with an immiscible solvent and separating the embedding agent from the sample; c) extracting the analyte from the de-embedded sample; and d) purifying the extracted analyte to provide an analyte sample. In some embodiments, the level of embedding agent in the analyte sample is reduced relative to a method wherein the embedding agent is dissolved with a miscible solvent, optionally wherein the miscible solvent is xylene, ethyl acetate, CitriSolv™, or UltraClear™. In some embodiments, the level of embedding agent in the analyte sample is reduced relative to a method wherein the embedding agent is removed by cutting the embedding agent from the embedded sample.

Further provided herein is a method of improving separation of embedding agent from an embedded sample, wherein the method comprises: a) providing an embedded sample comprising an analyte, wherein the sample is embedded in an embedding agent; b) removing the embedding agent from the embedded sample, thereby generating a de-embedded sample, wherein the embedding agent is removed by contacting the embedded sample with an immiscible solvent and separating the embedding agent from the sample; and c) extracting the analyte from the de-embedded sample. In some embodiments, the separation of embedding agent from the embedded sample is improved relative to a method wherein the embedding agent is dissolved with a miscible solvent, optionally wherein the miscible solvent is xylene, ethyl acetate, CitriSolv™, or UltraClear™. In some embodiments, the separation of embedding agent from the embedded sample is improved relative to a method wherein the embedding agent is removed by cutting the embedding agent from the embedded sample. In some embodiments, the method is performed in a liquid handling robot comprising a filter, wherein the improved separation of embedding agent results in less clogging of the filter relative to a method wherein the embedding agent is dissolved with a miscible solvent, optionally wherein the miscible solvent is xylene, ethyl acetate, CitriSolv™, or UltraClear™ In some embodiments, the method is performed in a liquid handling robot comprising a filter, wherein the improved separation of embedding agent results in less clogging of the filter relative to a method wherein the embedding agent is removed by cutting the embedding agent from the embedded sample. In some embodiments, the filter is a filter in a spin column.

In some embodiments of the preceding methods, step b) does not comprise dissolving the embedding agent with a miscible solvent, optionally wherein the miscible solvent is xylene, ethyl acetate, CitriSolv™, or UltraClear™. In some embodiments of the preceding methods, the density of the immiscible solvent is lighter than water and heavier than the embedding agent when the embedding agent is in liquid form. In some embodiments, the density of the immiscible solvent is heavier than liquid paraffin. In some embodiments of the preceding methods, the immiscible solvent is vegetable oil. In some embodiments of the preceding methods, the immiscible solvent is mineral oil. In some embodiments, the embedded sample is contacted with about 300, 350, 400, 450, 500, or 550 μL of mineral oil.

In some embodiments, the embedded sample is contacted with mineral oil at a mineral oil to embedded sample ratio of about 1:4, 1:2, 1:1, 2:1, 4:1, 5:1, 6:1, 7:1, 8:1, 9:1, 10:1, 15:1, 20:1, 30:1, 40:1, or 50:1. In some embodiments, the mineral oil contacts the embedded sample at about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., or about 75° C. In some embodiments, the mineral oil contacts the embedded sample for about 5 minutes, about 10 minutes, about 15 minutes, about 20 minutes, about 25 minutes, about 30 minutes, or about 35 minutes. In some embodiments, the mineral oil contacts the embedded sample while shaking at about 25 rpm, about 50 rpm, about 100 rpm, about 200 rpm, about 300 rpm, about 400 rpm, about 500 rpm, about 600 rpm, about 700 rpm, about 800 rpm, about 900 rpm, about 1000 rpm, or about 1100 rpm.

In some embodiments of the preceding methods, step b) comprises contacting the sample with an immiscible solvent, centrifuging, and filtering the embedded sample. In some embodiments, the embedded sample is centrifuged at about 1,700 rcf, about 1,750 rcf, about 1,800 rcf, about 1,850 rcf, or about 1,900 rcf. In some embodiments, the embedded sample is centrifuged at 1,811 rcf or greater. In some embodiments, the embedded sample is centrifuged at 1,811 rcf.

In some embodiments of the preceding methods, the analyte is selected from the group consisting of a polypeptide, RNA, DNA, a small molecule, a lipid, a polysaccharide, an exosome, a mitochondria, and a nucleus. In some embodiments of the preceding methods, the embedding agent is selected from the group consisting of paraffin, resin, celloidin, Paraplast®, gelatin, ester wax, wax, polyethylene glycol, and nitrocellulose. In some embodiments, the embedding agent is paraffin.

Further provided herein is a method of extracting RNA from a paraffin-embedded sample, wherein the method comprises: a) incubating the sample with a protease; b) incubating the sample at 50° C. to 75° C. for 1 to 40 minutes; c) centrifuging the sample at a high speed to separate the paraffin from a lysate comprising the RNA; and d) aspirating the lysate comprising the RNA. In some embodiments, the method further comprises cooling the sample to room temperature after step c) and before step d). In some embodiments, the method further comprises centrifuging the sample to filter the lysate following cooling the sample to room temperature. In some embodiments, incubating the sample with the protease comprises incubation at from 50° C. to 60° C. In some embodiments, incubating the sample with the protease comprises incubation for 1-20 minutes. In some embodiments, centrifuging the sample at high speed comprises centrifuging the sample at about 1,700 rcf, about 1,750 rcf, about 1,800 rcf, about 1,850 rcf, or about 1,900 rcf. In some embodiments, the RNA is isolated from the lysate. In some embodiments, the method of extracting RNA is used in a high throughput method. In some embodiments, the method further comprises analyzing the RNA. In some embodiments, the incubation with the protease is performed with shaking between 500 and 2000 rpm. In some embodiments, the incubation at 50° C. to 80° C. is performed without shaking. In some embodiments, the method further comprises centrifuging the sample at from 250 RCF to 750 RCF following step c). In some embodiments, the method further comprises preparing cDNA from the RNA.

Further provided herein is a method of extracting DNA from a sample, wherein the method comprises a) incubating the sample with a protease; b) incubating the sample at 50° C. to 80° C. for 2-48 hours; c) centrifuging the sample at high speed to produce a lysate comprising the DNA; and d) aspirating the lysate comprising the DNA. In some embodiments, incubating the sample at 50° C. to 80° C. for 2-48 hours is performed while shaking at between 500 and 2000 rpm. In some embodiments, immediately following step b), the sample is centrifuged for 2 to 20 minutes at from 1000 RCF to 4000 RCF. In some embodiments, the sample is refrigerated for at least 40 minutes following centrifugation for 2 to 20 minutes at from 1000 RCF to 4000 RCF. In some embodiments, step c) is carried out after refrigerating the sample. In some embodiments, centrifuging the sample at high speed comprises centrifuging the sample at about 1,700 rcf, about 1,750 rcf, about 1,800 rcf, about 1,850 rcf, or about 1,900 rcf.

Further provided herein is a method of extracting RNA and DNA from a paraffin-embedded sample, wherein the method comprises: a) incubating the sample with a protease; b) incubating the sample at 50° C. to 80° C. for 1 to 40 minutes; c) centrifuging the sample at a high speed to separate the paraffin from a first lysate comprising the RNA; d) aspirating the first lysate comprising the RNA; e) isolating the RNA from the first lysate; f) incubating the sample from step c) with a protease; g) incubating the sample from step f) at 50° C. to 80° C. for 2-48 hours; h) centrifuging the sample at high speed; i) aspirating a second lysate comprising the DNA; j) isolating the DNA from the second lysate. In some embodiments, centrifuging the sample at high speed comprises centrifuging the sample at about 1,700 rcf, about 1,750 rcf, about 1,800 rcf, about 1,850 rcf, or about 1,900 rcf. In some embodiments, incubation with the protease is performed while shaking at between 500 and 2000 rpm in step a and/or step f. In some embodiments, incubating the sample with the protease comprises incubation at from 50° C. to 60° C. In some embodiments, incubating the sample with the protease comprises incubation for 1-20 minutes. In some embodiments, incubating the sample at 50° C. to 80° C. for 1 to 40 minutes is performed without shaking.

Further provided herein is a method of extracting RNA from a paraffin-embedded sample, wherein the method comprises: a) adding mineral oil to the sample; b) incubating the sample at 50° C. to 80° C. for 1 to 40 minutes; c) cooling the sample to room temperature; d) incubating the sample with a protease; f) incubating the sample at 50° C. to 80° C. for 1 to 40 minutes; g) centrifuging the sample at a high speed to separate the paraffin and the mineral oil from a lysate comprising the RNA; and h) aspirating the lysate comprising the RNA. In some embodiments, the incubation at 50° C. to 80° C. for 1 to 20 minutes is performed with shaking at from 500 RPM to 2000 RPM. In some embodiments, centrifuging the sample at high speed comprises centrifuging the sample at about 1,700 rcf, about 1,750 rcf, about 1,800 rcf, about 1,850 rcf, or about 1,900 rcf. In some embodiments, the sample is incubated at room temperature following centrifugation at high speed. In some embodiments, the method further comprises centrifuging the sample after incubation at room temperature.

Further provided herein is a method of extracting DNA from a paraffin-embedded sample, wherein the method comprises: a) adding mineral oil to the sample; b) incubating the sample with a protease; c) incubating the sample at 50° C. to 80° C. for 2-48 hours; d) centrifuging the sample at a high speed to separate the paraffin from a lysate comprising the DNA; and e) aspirating the lysate comprising the DNA. In some embodiments, the mineral oil is removed prior to step b). In some embodiments, centrifuging the sample at high speed comprises centrifuging the sample at about 1,700 rcf, about 1,750 rcf, about 1,800 rcf, about 1,850 rcf, or about 1,900 ref. In some embodiments, incubating the sample at 50° C. to 80° C. for 2-48 hours is performed with shaking at from 500 RPM to 2000 RPM. In some embodiments, following incubating the sample at 50° C. to 80° C. for 2-48 hours, the sample is immediately centrifuged at 2000 to 5000 RCF prior to step d).

Further provided herein is a method of extracting RNA and DNA from a paraffin-embedded sample, wherein the method comprises: a) adding mineral oil to the sample; b) melting the paraffin by incubating the sample at 50° C. to 80° C. for 1 to 40 minutes; c) cooling the sample to room temperature; d) incubating the sample with a protease; e) incubating the sample at 50° C. to 80° C. for 1 to 40 minutes; f) centrifuging the sample at a high speed to separate the paraffin and the mineral oil from a lysate comprising the RNA; g) aspirating the lysate comprising the RNA; h) isolating the RNA from the lysate; i) centrifuging the lysate from step g) at a high speed to separate the lysate from the mineral oil; j) incubating the sample from step i) with a protease; k) incubating the sample from step j) at 50° C. to 80° C. for 2-48 hours; 1) centrifuging the sample at high speed; and m) aspirating a second lysate comprising the DNA. In some embodiments, centrifuging the sample at high speed comprises centrifuging the sample at about 1,700 rcf, about 1,750 rcf, about 1,800 rcf, about 1,850 rcf, or about 1,900 rcf. In some embodiments, incubating the sample at 50° C. to 80° C. for 2-48 hours is performed with shaking at from 500 RPM to 2000 RPM.

In some embodiments of the preceding methods, the protease is proteinase K. In some embodiments of the preceding methods, the sample is less than about 30 μm³ in size. In some embodiments of the preceding methods, the sample is about 0.3 μm³ to about 5.5 μm³ in size. In some embodiments of the preceding methods, the method further comprises analyzing the RNA and/or the DNA extracted from the sample by one or more methods selected from the group consisting of a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), and mass-spectrometric genotyping. In some embodiments, analyzing the RNA and/or DNA comprises next generation sequencing. In some embodiments of the preceding methods, the method further comprises preparing a sequencing library for sequencing the RNA and/or the DNA In some embodiments of the preceding methods, the method further comprises sequencing the DNA and/or RNA using hybrid capture based sequencing. In some embodiments of the preceding methods, the sample is from an individual known to have cancer or suspected of having cancer.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 provides an overview of exemplary RNA-first (top) and DNA-first (bottom) methods of extracting RNA and DNA from paraffin-embedded samples.

FIG. 2 shows a representative image of a turbid nucleic acid sample extracted from a paraffin-embedded tissue following centrifugation at 10,000 rcf for 2 minutes. The arrow indicates the pellet containing wax (paraffin) contamination.

FIG. 3A shows a schematic diagram showing the three arms of the experiments used to test the RNA-first extraction workflow. Arm 2 is the standard protocol (left column), arm 3 is the oil method (center column), and arm 4 is the warm lysate spin method (right column). FIG. 3B shows a schematic diagram showing the three arms of the experiments used to test the DNA-first extraction workflow. Arm 2 is the standard protocol (left column), arm 3 is the oil method (center column), and arm 4 is the warm lysate spin method (right column).

FIG. 4 shows an overview of an exemplary method for performing precision enrichment of pathology specimens from a formalin-fixed paraffin-embedded (FFPE) block.

FIGS. 5A-5B provides an overview of exemplary comprehensive genomic profiling (CGP) methods.

FIG. 6 depicts an exemplary device, “Device 1100,” in accordance with some embodiments.

FIG. 7 depicts an exemplary system, “System 1200,” in accordance with some embodiments.

FIG. 8 depicts a block diagram of an exemplary process for analyzing RNA and/or DNA extracted from a paraffin-embedded sample, in accordance with some embodiments.

FIG. 9A and FIG. 9B depict AutoLys tubes after warm lysate process. In FIG. 9A, the inner AutoLys tube is shown on the left and the arrow shows a paraffin layer that remains in the inner tube; the outer tube is shown on the right and the arrow shows a clear eluate with minimal residual paraffin in the lysate. In FIG. 9B, the inner AutoLys tubes are shown on the top and the arrows show a paraffin layer that remains in the inner tubes; the outer tubes are shown on the bottom and the arrows show a clear eluate with minimal residual paraffin in the lysates.

FIG. 10A and FIG. 10B depict the results of a mineral oil extraction process. In FIG. 10A, the white arrow identifies the oil layer while the black arrow identifies the lysate layer that is clear and has minimal residual paraffin. In FIG. 10B, the left two columns were extracted using the mineral oil method while the right two columns were extracted using the “standard” method; the image shows a clear eluate with minimal residual paraffin for the samples extracted using the mineral oil method and turbid eluate with more residual paraffin for the samples extracted using the standard method.

### DETAILED DESCRIPTION

The present disclosure is based, at least in part, on the development of methods for extracting nucleic acids from tissues that involves a histologic quality assurance/quality control step to assess the enrichment process. The methods described herein enable testing of specimens that otherwise would produce a level of tissue that was insufficient for analysis of tumor content with prior enrichment methods.

In some embodiments, the present methods have the advantage of being able to extract DNA and RNA from small samples, such as on the order of 30 μM² or smaller. Moreover, the present methods can in some embodiments be used to extract DNA and RNA from the same sample. Furthermore, in some embodiments the methods relate to removal of paraffin from a sample, which allows for more efficient downstream processing, specifically in high throughput methods. For example, in some embodiments, removal of paraffin using the present methods can decrease clogging of robots used in a high throughput sample processing method.

### Definitions

Before describing the invention in detail, it is to be understood that this invention is not limited to particular compositions or biological systems. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “or” is used herein to mean, and is used interchangeably with, the term “and/or”, unless context clearly indicates otherwise.

The terms “about” or “approximately” as used herein refer to the usual error range for the respective value readily known to the skilled person in this technical field, for example, an acceptable degree of error or deviation for the quantity measured given the nature or precision of the measurements. Reference to “about” or “approximately” a value or parameter herein includes (and describes) embodiments that are directed to that value or parameter per se.

As used herein, the term “embedding agent” refers to any agent that may be used to embed a sample, such as a tissue sample. Exemplary embedding agents include paraffin, resin celloidin, Paraplast®, gelatin, ester wax, wax, polyethylene glycol, and nitrocellulose. In particular embodiments, the embedding agent is paraffin.

As used herein, the term “embedded sample” refers to any sample that is embedded in an embedding agent. In some embodiments, the embedded sample is an embedded tissue sample, such as an embedded tissue sample extracted from an individual known to have cancer or suspected of having cancer. In particular embodiments, the embedded sample is a paraffin-embedded sample.

As used herein, the term “analyte” refers to any molecule that may be extracted from an embedded sample. Exemplary analytes include RNA, DNA, polypeptides, small molecules, lipids, polysaccharides, exosomes, mitochondria, and nuclei.

As used herein, the term “configured to hybridize to” indicates that a nucleic acid molecule has a nucleotide sequence with sufficient length and sequence complementarity to the nucleotide sequence of a target nucleic acid to allow the nucleic acid molecule to hybridize to the target nucleic acid, e.g., with a Tₘ of at least 65° C. in an aqueous solution of 1×SCC (150 mM sodium chloride and 15 mM trisodium citrate) and 0.1% SDS. Other hybridization conditions may be used when hybridizing a nucleic acid molecule to a target nucleic acid molecule, for example in the context of a described method.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human. In some embodiments, the individual is human patient, e.g., a human patient having a cancer described herein.

An “effective amount” or a “therapeutically effective amount” of an agent, e.g., an anti-cancer agent, or a pharmaceutical formulation, refers to an amount effective, at dosages and for periods of time necessary, to achieve the desired therapeutic or prophylactic result, e.g., in the treatment or management of a cancer, for example, delaying or minimizing one or more symptoms associated with the cancer. In some embodiments, an effective amount or a therapeutically effective amount of an agent refers to an amount of the agent at dosages and for periods of time necessary, alone or in combination with other therapeutic agents, which provides a therapeutic or prophylactic benefit in the treatment or management of a disease such as a cancer. In some embodiments, an effective amount or a therapeutically effective amount of an agent enhances the therapeutic or prophylactic efficacy of another therapeutic agent or another therapeutic modality.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, delaying progression of disease, alleviation of symptoms, diminishment of any direct or indirect pathological consequences of the disease, decreasing the rate of disease progression, amelioration or palliation of the disease state, and remission or improved prognosis. In some embodiments, the terms “treatment,” “treat,” or “treating” include preventing a disease, such as cancer, e.g., before an individual begins to suffer from a cancer or from re-growth or recurrence of the cancer. In some embodiments, the terms “treatment,” “treat,” or “treating” include inhibiting or reducing the severity of a disease such as a cancer.

“Likely to” or “increased likelihood,” as used herein, refer to an increased probability that an event, item, object, thing or person will occur. Thus, in one example, an individual that is likely to respond to treatment with an anti-cancer therapy, e.g., an anti-cancer therapy provided herein, alone or in combination, has an increased probability of responding to treatment with the anti-cancer therapy alone or in combination, relative to a reference individual or group of individuals. “Unlikely to” refers to a decreased probability that an event, item, object, thing or person will occur relative to a reference individual or group of individuals. Thus, an individual that is unlikely to respond to treatment with an anti-cancer therapy, e.g., an anti-cancer therapy provided herein, alone or in combination, has a decreased probability of responding to treatment with the anti-cancer therapy, alone or in combination, relative to a reference individual or group of individuals.

### I. Removing Embedding Agents From Embedded Samples

In some aspects, provided herein are methods of detecting an analyte, wherein the method comprises: providing an embedded sample comprising an analyte, wherein the sample is embedded in an embedding agent; removing the embedding agent from the embedded sample, thereby generating a de-embedded sample, wherein the embedding agent is removed by inducing a phase transition in the embedding agent and separating the embedding agent from the sample; extracting the analyte from the de-embedded sample; and analyzing the analyte to detect the analyte. Also provided herein are methods of extracting an analyte, wherein the method comprises: providing an embedded sample comprising an analyte, wherein the sample is embedded in an embedding agent; removing the embedding agent from the embedded sample, thereby generating a de-embedded sample, wherein the embedding agent is removed by inducing a phase transition in the embedding agent and separating the embedding agent from the sample; and extracting the analyte from the de-embedded sample. Also provided herein are methods of reducing the level of embedding agent in an analyte sample extracted from an embedded sample, wherein the method comprises: providing an embedded sample comprising an analyte, wherein the sample is embedded in an embedding agent; removing the embedding agent from the embedded sample, thereby generating a de-embedded sample, wherein the embedding agent is removed by inducing a phase transition in the embedding agent and separating the embedding agent from the sample; extracting the analyte from the de-embedded sample; and purifying the extracted analyte to provide an analyte sample. Also provided herein are methods of improving separation of an embedding agent from an embedded sample, wherein the method comprises: providing an embedded sample comprising an analyte, wherein the sample is embedded in an embedding agent; removing the embedding agent from the embedded sample, thereby generating a de-embedded sample, wherein the embedding agent is removed by inducing a phase transition in the embedding agent and separating the embedding agent from the sample; and extracting an analyte from the de-embedded sample. Also provided herein are methods of detecting an analyte, wherein the method comprises: providing an embedded sample comprising an analyte, wherein the sample is embedded in an embedding agent; removing the embedding agent from the embedded sample, thereby generating a de-embedded sample, wherein the embedding agent is removed by contacting the embedded sample with an immiscible solvent and separating the embedding agent from the sample; extracting the analyte from the de-embedded sample; and analyzing the analyte to detect the analyte. Also provided herein are methods of extracting an analyte, wherein the method comprises: providing an embedded sample comprising an analyte, wherein the sample is embedded in an embedding agent; removing the embedding agent from the embedded sample, thereby generating a de-embedded sample, wherein the embedding agent is removed by contacting the embedded sample with an immiscible solvent and separating the embedding agent from the sample; and extracting the analyte from the de-embedded sample. Also provided herein are methods of reducing the level of embedding agent in an analyte sample extracted from an embedded sample, wherein the method comprises: providing an embedded sample comprising an analyte, wherein the sample is embedded in an embedding agent; removing the embedding agent from the embedded sample, thereby generating a de-embedded sample, wherein the embedding agent is removed by contacting the embedded sample with an immiscible solvent and separating the embedding agent from the sample; extracting the analyte from the de-embedded sample; and purifying the extracted analyte to provide an analyte sample. Also provided herein are methods of improving separation of embedding agent from an embedded sample, wherein the method comprises: providing an embedded sample comprising an analyte, wherein the sample is embedded in an embedding agent; removing the embedding agent from the embedded sample, thereby generating a de-embedded sample, wherein the embedding agent is removed by contacting the embedded sample with an immiscible solvent and separating the embedding agent from the sample; and extracting the analyte from the de-embedded sample. Also provided herein are methods of detecting alterations in RNA and/or DNA, wherein the method comprises: providing a paraffin-embedded sample; removing paraffin from the paraffin-embedded sample, thereby generating a deparaffinized sample, wherein the paraffin is removed by inducing a phase transition in the paraffin and separating the paraffin from the sample; extracting RNA and/or DNA from the deparaffinized sample; and analyzing the RNA and/or DNA to detect alterations in the RNA and/or DNA. Also provided herein are methods of extracting RNA and/or DNA, wherein the method comprises: providing a paraffin-embedded sample; removing paraffin from the paraffin-embedded sample, thereby generating a deparaffinized sample, wherein the paraffin is removed by inducing a phase transition in the paraffin and separating the paraffin from the sample; and extracting RNA and/or DNA from the deparaffinized sample. Also provided herein are methods of improving library construction for nucleic acid sequencing, wherein the method comprises: providing a paraffin-embedded sample; removing paraffin from the paraffin-embedded sample, thereby generating a deparaffinized sample, wherein the paraffin is removed by inducing a phase transition in the paraffin and separating the paraffin from the sample; extracting RNA and/or DNA from the deparaffinized sample; and preparing a sequencing library for sequencing the RNA and/or the DNA. Also provided herein are methods of reducing the level of paraffin in an RNA or DNA sample extracted from a paraffin-embedded sample, wherein the method comprises: providing a paraffin-embedded sample; removing paraffin from the paraffin-embedded sample, thereby generating a deparaffinized sample, wherein the paraffin is removed by inducing a phase transition in the paraffin and separating the paraffin from the sample; extracting RNA and/or DNA from the deparaffinized sample; and purifying the extracted RNA and/or DNA to provide an RNA and/or DNA sample. Also provided herein are methods of improving separation of paraffin from a paraffin-embedded sample, wherein the method comprises: providing a paraffin-embedded sample; removing paraffin from the paraffin-embedded sample, thereby generating a deparaffinized sample, wherein the paraffin is removed by inducing a phase transition in the paraffin and separating the paraffin from the sample; and extracting RNA and/or DNA from the deparaffinized sample. Also provided herein are methods of detecting alterations in RNA and/or DNA, wherein the method comprises: providing a paraffin-embedded sample; removing paraffin from the paraffin-embedded sample, thereby generating a deparaffinized sample, wherein the paraffin is removed by contacting the paraffin-embedded sample with an immiscible solvent and separating the paraffin from the sample; extracting RNA and/or DNA from the deparaffinized sample; and analyzing the RNA and/or DNA to detect alterations in the RNA and/or DNA. A method of extracting RNA and/or DNA, wherein the method comprises: providing a paraffin-embedded sample; removing paraffin from the paraffin-embedded sample, thereby generating a deparaffinized sample, wherein the paraffin is removed by contacting the paraffin-embedded sample with an immiscible solvent and separating the paraffin from the sample; and extracting RNA and/or DNA from the deparaffinized sample. Also provided herein are methods of improving library construction for nucleic acid sequencing, wherein the method comprises: providing a paraffin-embedded sample; removing paraffin from the paraffin-embedded sample, thereby generating a deparaffinized sample, wherein the paraffin is removed by contacting the paraffin-embedded sample with an immiscible solvent and separating the paraffin from the sample; extracting RNA and/or DNA from the deparaffinized sample; and preparing a sequencing library for sequencing the RNA and/or the DNA. Also provided herein are methods of reducing the level of paraffin in an RNA or DNA sample extracted from a paraffin-embedded sample, wherein the method comprises: providing a paraffin-embedded sample; removing paraffin from the paraffin-embedded sample, thereby generating a deparaffinized sample, wherein the paraffin is removed by contacting the paraffin-embedded sample with an immiscible solvent and separating the paraffin from the sample; extracting RNA and/or DNA from the deparaffinized sample; and purifying the extracted RNA and/or DNA to provide an RNA and/or DNA sample. Also provided herein are methods of improving separation of paraffin from a paraffin-embedded sample, wherein the method comprises: providing a paraffin-embedded sample; removing paraffin from the paraffin-embedded sample, thereby generating a deparaffinized sample, wherein the paraffin is removed by contacting the paraffin-embedded sample with an immiscible solvent and separating the paraffin from the sample; and extracting RNA and/or DNA from the deparaffinized sample.

The present disclosure is based, at least in part, on the development of methods for separating embedded samples from embedding agents. For example, the methods described herein may be used to separate paraffin from paraffin-embedded samples such as formalin-fixed paraffin-embedded (FFPE) tissues. Without wishing to be bound by any particular theory, it is believed that, following removal of an embedding agent from an embedded sample, poor separation of embedding agents presents various problems that emerge during the subsequent analysis of analytes extracted from the samples. For example, poor separation of embedding agents from samples extracted from de-embedded may be an impurity in an analyte sample extracted from a de-embedded sample. In the case of paraffin, this can result in turbid preparations of analyte samples and clogging of liquid handling robots that are used to process the analyte samples. In the case of RNA and/or DNA samples extracted from de-paraffinized samples, poor removal of paraffin can result in failures in library construction for next-generation sequence analysis.

Importantly, the methods described herein do not rely on the use of toxic solvents such as xylene, and are therefore amenable to automatable and high-throughput analyses, such as the high throughput preparation of libraries for sequencing nucleic acids.

### Inducing a Phase Transition in an Embedding Agent

In some embodiments, provided herein are methods involving inducing a phase transition an embedding agent in order to remove an embedding agent from an embedded sample, thereby generating a de-embedded sample. The embedding agent may be any one of the embedding agents described herein. The methods described herein may be used to separate embedding agents from embedded sample. In some embodiments, the methods described herein further comprise extracting an analyte from the de-embedded sample. The analyte may be any one of the analytes described herein.

In some embodiments, provided herein is a method of detecting an analyte, wherein the method comprises: providing an embedded sample comprising an analyte, wherein the sample is embedded in an embedding agent; removing the embedding agent from the embedded sample, thereby generating a de-embedded sample, wherein the embedding agent is removed by inducing a phase transition in the embedding agent and separating the embedding agent from the sample; extracting the analyte from the de-embedded sample; and analyzing the analyte to detect the analyte. In some embodiments, the detection the analyte is improved relative to a method wherein the embedding agent is dissolved with a miscible solvent (e.g., xylene, ethyl acetate, CitriSolv™ or UltraClear™). In some embodiments, the detection the analyte is improved relative to a method wherein the embedding agent is removed by cutting the embedding agent from the embedded sample. In some embodiments, the detection the analyte is improved relative to a method wherein the embedded sample is not de-embedded.

In some embodiments, provided herein is a method of extracting an analyte, wherein the method comprises: providing an embedded sample comprising an analyte, wherein the sample is embedded in an embedding agent; removing the embedding agent from the embedded sample, thereby generating a de-embedded sample, wherein the embedding agent is removed by inducing a phase transition in the embedding agent and separating the embedding agent from the sample; and extracting the analyte from the de-embedded sample. In some embodiments, the extraction of the analyte is improved relative to a method wherein the embedding agent is dissolved with a miscible solvent (e.g., xylene, ethyl acetate, CitriSolv™, or UltraClear™). In some embodiments, the extraction of the analyte is improved relative to a method wherein the embedding agent is removed by cutting the embedding agent from the embedded sample.

In some embodiments, provided herein is a method of reducing the level of embedding agent in an analyte sample extracted from an embedded sample, wherein the method comprises: providing an embedded sample comprising an analyte, wherein the sample is embedded in an embedding agent; removing the embedding agent from the embedded sample, thereby generating a de-embedded sample, wherein the embedding agent is removed by inducing a phase transition in the embedding agent and separating the embedding agent from the sample; extracting the analyte from the de-embedded sample; and purifying the extracted analyte to provide an analyte sample. In some embodiments, the level of embedding agent in the analyte sample is reduced relative to a method wherein the embedding agent is dissolved with a miscible solvent (e.g., xylene, ethyl acetate, CitriSolv™, or UltraClear™). In some embodiments, the level of embedding agent in the analyte sample is reduced relative to a method wherein the embedding agent is removed by cutting the embedding agent from the embedded sample. The level of embedding agent in the analyte sample may be measured, for example, by measuring the turbidity of the analyte sample. In some embodiments, the turbidity of the analyte sample is reduced relative to a method wherein the embedding agent is dissolved with a miscible solvent and/or relative to a method wherein the embedding agent is removed by cutting the embedding agent from the embedded sample.

In some embodiments, provided herein is a method of improving separation of an embedding agent from an embedded sample, wherein the method comprises: providing an embedded sample comprising an analyte, wherein the sample is embedded in an embedding agent; removing the embedding agent from the embedded sample, thereby generating a de-embedded sample, wherein the embedding agent is removed by inducing a phase transition in the embedding agent and separating the embedding agent from the sample; and extracting an analyte from the de-embedded sample. In some embodiments, the separation of embedding agent from the embedded sample is improved relative to a method wherein the embedding agent is dissolved with a miscible solvent (e.g., xylene, ethyl acetate, CitriSolv™, or UltraClear™). In some embodiments, the separation of embedding agent from the embedded sample is improved relative to a method wherein the embedding agent is removed by cutting the embedding agent from the embedded sample. In some embodiments, the method is performed in a liquid handling robot comprising a filter, wherein the improved separation of embedding agent results in less clogging of the filter relative to a method wherein the embedding agent is dissolved with a miscible solvent (e.g., xylene, ethyl acetate, CitriSolv™, or UltraClear™). As described above, the level of embedding agent in the analyte sample may be measured, for example, by measuring the turbidity of the analyte sample. In some embodiments, the turbidity of the analyte sample is reduced relative to a method wherein the embedding agent is dissolved with a miscible solvent and/or relative to a method wherein the embedding agent is removed by cutting the embedding agent from the embedded sample. In some embodiments, the method is performed in a liquid handling robot comprising a filter, wherein the improved separation of embedding agent results in less clogging of the filter relative to a method wherein the embedding agent is removed by cutting the embedding agent from the embedded sample. In some embodiments, the filter is a filter in a spin column. In some embodiments, the liquid handling robot is a Hamilton AutoLys STAR, and the spin column is an AutoLys tube.

In some embodiments of any of the methods described above, step b) does not comprise dissolving the embedding agent with a miscible solvent. In some embodiments, the miscible solvent is xylene, ethyl acetate, CitriSolv™, or UltraClear™.

In some embodiments, the phase transition is selected from the group consisting of melting, freezing, vaporization, condensation, sublimation, and deposition. In some embodiments, step b) comprises heating, cooling, increasing the pressure, or decreasing the pressure of the embedded sample.

In some embodiments, the phase transition is melting. In some embodiments, step b) comprises removing the embedding agent from the embedded sample by melting the embedding agent, thereby generating a de-embedded sample. In some embodiments, step b) comprises melting the embedding agent and separating the embedding agent from the sample. In some embodiments, step b) comprises heating the embedded sample. In some embodiments, the embedded sample is heated to about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., or about 75° C. In some embodiments, the embedded sample is heated for about 5 minutes, about 10 minutes, about 15 minutes, about 20 minutes, about 25 minutes, about 30 minutes, or about 35 minutes.

In some embodiments, step b) comprises centrifuging and filtering the embedded sample to separate the embedding from the sample. In some embodiments, the embedded sample is centrifuged at about 1,700 rcf, about 1,750 rcf, about 1,800 rcf, about 1,850 rcf, or about 1,900 rcf. In some embodiments, the embedded sample is centrifuged at 1,811 rcf or greater. In some embodiments, the embedded sample is centrifuged at 1,811 rcf. In some embodiments, the centrifuging and filtering is performed in a liquid handling robot, e.g., a Hamilton AutoLys STAR.

In some embodiments, step b) comprises heating, centrifuging, and filtering the embedded sample to separate the embedding agent from the sample, thereby generating a de-embedded sample. In some embodiments, the heating, centrifuging and filtering are performed as described above. In some embodiments, the heating, centrifuging and filtering is performed in a liquid handling robot, e.g., a Hamilton AutoLys STAR.

### Methods Involving Paraffin-Embedded Samples

In some embodiments, the embedding agent is paraffin. Accordingly, in some embodiments, provided herein are methods involving removing paraffin from paraffin-embedded samples. Exemplary methods involving removing paraffin from paraffin-embedded samples using methods involving phase transition are described herein in the Examples.

In some embodiments, provided herein is a method of detecting alterations in RNA and/or DNA, wherein the method comprises: providing a paraffin-embedded sample; removing paraffin from the paraffin-embedded sample, thereby generating a deparaffinized sample, wherein the paraffin is removed by inducing a phase transition in the paraffin and separating the paraffin from the sample; extracting RNA and/or DNA from the deparaffinized sample; and analyzing the RNA and/or DNA to detect alterations in the RNA and/or DNA. In some embodiments, the detection of alterations in the RNA and/or DNA is improved relative to a method wherein the paraffin is dissolved with a miscible solvent. In some embodiments, the miscible solvent is xylene, ethyl acetate, CitriSolv™, or UltraClear™. In some embodiments, the detection of alterations in the RNA and/or DNA is improved relative to a method wherein the paraffin is removed by cutting the paraffin from the paraffin-embedded sample. In some embodiments, the detection of alterations in the RNA and/or DNA is improved relative to a method wherein the paraffin-embedded sample is not deparaffinized. In some embodiments, the alteration in the RNA and/or DNA is selected from the group consisting of: a copy number alteration; a point mutation; an in-frame deletion of one or more codons; an intragenic deletion; an intragenic insertion; a deletion of a full gene; an inversion; an interchromosomal translocation; a tandem duplication; a gene fusion; a genomic rearrangement that comprises an intron sequence; and/or a gene amplification or duplication. In particular embodiments, the alteration in the RNA and/or the DNA is a copy number alteration. Methods of detecting alterations in RNA and/or DNA are described herein in detail below.

In some embodiments, provided herein is a method of extracting RNA and/or DNA, wherein the method comprises: providing a paraffin-embedded sample; removing paraffin from the paraffin-embedded sample, thereby generating a deparaffinized sample, wherein the paraffin is removed by inducing a phase transition in the paraffin and separating the paraffin from the sample; and extracting RNA and/or DNA from the deparaffinized sample. In some embodiments, the extraction of the RNA and/or DNA is improved relative to a method wherein the paraffin is dissolved with a miscible solvent. In some embodiments, the miscible solvent is xylene, ethyl acetate, CitriSolv™, or UltraClear™. In some embodiments, the extraction of the RNA and/or DNA is improved relative to a method wherein the paraffin is removed by cutting the paraffin from the paraffin-embedded sample. Methods of extracting RNA and/or DNA are described herein in detail below.

In some embodiments, provided herein is a method of improving library construction for nucleic acid sequencing, wherein the method comprises: providing a paraffin-embedded sample; removing paraffin from the paraffin-embedded sample, thereby generating a deparaffinized sample, wherein the paraffin is removed by inducing a phase transition in the paraffin and separating the paraffin from the sample; extracting RNA and/or DNA from the deparaffinized sample; and preparing a sequencing library for sequencing the RNA and/or the DNA. In some embodiments, the preparation of the sequencing library is improved relative to a method wherein the paraffin is dissolved with a miscible solvent. In some embodiments, the miscible solvent is xylene, ethyl acetate, CitriSolv™, or UltraClear™. In some embodiments, the preparation of the sequencing library is improved relative to a method wherein the paraffin is removed by cutting the paraffin from the paraffin-embedded sample.

In some embodiments, provided herein is a method of reducing the level of paraffin in an RNA or DNA sample extracted from a paraffin-embedded sample, wherein the method comprises: providing a paraffin-embedded sample; removing paraffin from the paraffin-embedded sample, thereby generating a deparaffinized sample, wherein the paraffin is removed by inducing a phase transition in the paraffin and separating the paraffin from the sample; extracting RNA and/or DNA from the deparaffinized sample; and purifying the extracted RNA and/or DNA to provide an RNA and/or DNA sample. In some embodiments, the level of paraffin in the RNA and/or DNA sample is reduced relative to a method wherein the paraffin is dissolved with a miscible solvent. In some embodiments, the miscible solvent is xylene, ethyl acetate, CitriSolv™, or UltraClear™. In some embodiments, the level of paraffin in the RNA and/or DNA sample is reduced relative to a method wherein the paraffin is removed by cutting the paraffin from the paraffin-embedded sample. The level of paraffin in the RNA and/or DNA sample may be measured, for example, by measuring the turbidity of the RNA and/or DNA sample. In some embodiments, the turbidity of the RNA and/or DNA sample is reduced relative to a method wherein the paraffin is dissolved with a miscible solvent and/or relative to a method wherein the paraffin is removed by cutting the paraffin from the embedded sample. In some embodiments, the turbidity of an RNA and/or DNA sample prepared by a method wherein the paraffin is dissolved with a miscible solvent and/or a method wherein the paraffin is removed by cutting the paraffin from the paraffin-embedded sample is 2-fold, 3-fold, 4-fold, 5-fold, 6-fold, 7-fold, 8-fold, 9-fold, 10-fold, 20-fold, 30-fold, 40-fold, or 50-fold the turbidity of an RNA and/or DNA sample prepared using a method wherein the paraffin is removed by inducing a phase transition in the paraffin and separating the paraffin from the sample.

In some embodiments, provided herein is a method of improving separation of paraffin from a paraffin-embedded sample, wherein the method comprises: providing a paraffin-embedded sample; removing paraffin from the paraffin-embedded sample, thereby generating a deparaffinized sample, wherein the paraffin is removed by inducing a phase transition in the paraffin and separating the paraffin from the sample; and extracting RNA and/or DNA from the deparaffinized sample. In some embodiments, the separation of paraffin from the paraffin-embedded sample is improved relative to a method wherein the paraffin is dissolved with a miscible solvent. In some embodiments, the miscible solvent is xylene, ethyl acetate, CitriSolv™ or UltraClear™. In some embodiments, the separation of paraffin from the paraffin-embedded sample is improved relative to a method wherein the paraffin is removed by cutting the paraffin from the paraffin-embedded sample. As described above, the level of paraffin in the RNA and/or DNA sample may be measured, for example, by measuring the turbidity of the RNA and/or DNA sample. In some embodiments, a measurement of the turbidity of the RNA and/or DNA sample indicates that separation of the paraffin is improved relative to a method wherein the paraffin is dissolved with a miscible solvent and/or relative to a method wherein the paraffin is removed by cutting the paraffin from the paraffin-embedded sample. In some embodiments, the turbidity of an RNA and/or DNA sample prepared by a method wherein the paraffin is dissolved with a miscible solvent and/or a method wherein the paraffin is removed by cutting the paraffin from the paraffin-embedded sample is 2-fold, 3-fold, 4-fold, 5-fold, 6-fold, 7-fold, 8-fold, 9-fold, 10-fold, 20-fold, 30-fold, 40-fold, or 50-fold the turbidity of an RNA and/or DNA sample prepared using a method wherein the paraffin is removed by inducing a phase transition in the paraffin and separating the paraffin from the sample. In some embodiments, the method is performed in a liquid handling robot comprising a filter, wherein the improved separation of paraffin results in less clogging of the filter relative to a method wherein the paraffin is dissolved with a miscible solvent. In some embodiments, the miscible solvent is xylene, ethyl acetate, CitriSolv™, or UltraClear™. In some embodiments, the method is performed in a liquid handling robot comprising a filter, wherein the improved separation of paraffin results in less clogging of the filter relative to a method wherein the paraffin is removed by cutting the paraffin from the paraffin-embedded sample. In some embodiments, the filter is a filter in a spin column. In some embodiments, the liquid handling robot is a Hamilton AutoLys STAR, and the spin column is an AutoLys tube.

In some embodiments of any one of the preceding methods in which the embedded sample is a paraffin-embedded sample, step b) does not comprise dissolving the paraffin with a miscible solvent. In some embodiments, the miscible solvent is xylene, ethyl acetate, CitriSolv™ or UltraClear™. In some embodiments of any one of the preceding methods in which the embedded sample is a paraffin-embedded sample, step b) does not comprise dissolving the paraffin with a toxic solvent.

In some embodiments, step b) comprises heating the paraffin-embedded sample. In some embodiments, the paraffin-embedded sample is heated to about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., or about 75° C. In some embodiments, the paraffin-embedded sample is heated for about 5 minutes, about 10 minutes, about 15 minutes, about 20 minutes, about 25 minutes, about 30 minutes, or about 35 minutes.

In some embodiments, step b) comprises centrifuging and filtering the paraffin-embedded sample. In general, the sample is centrifuged and filtered in such a way to separate the paraffin from the embedded sample based on the relative density of the paraffin and the sample, as well as the presence of particles in the sample. In some embodiments, the paraffin-embedded sample is centrifuged at about 1,700 rcf, about 1,750 rcf, about 1,800 rcf, about 1,850 rcf, or about 1,900 rcf. In some embodiments, the paraffin-embedded sample is centrifuged at 1,811 rcf or greater. In some embodiments, the paraffin-embedded sample is centrifuged at 1,811 rcf.

In some embodiments, step b) comprises heating and centrifuging the paraffin-embedded sample. In some embodiments, the paraffin-embedded sample is heated to about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., or about 75° C. The method of claim 31 or claim 32, wherein the paraffin-embedded sample is heated for about 5 minutes, about 10 minutes, about 15 minutes, about 20 minutes, about 25 minutes, about 30 minutes, or about 35 minutes. The method of any one claims 31-33, wherein the paraffin-embedded sample is centrifuged at about 1,700 rcf, about 1,750 rcf, about 1,800 rcf, about 1,850 rcf, or about 1,900 rcf. The method of any one claims 31-34, wherein the paraffin-embedded sample is centrifuged at 1,811 rcf or greater. The method of any one claims 31-35, wherein the paraffin-embedded sample is centrifuged at 1,811 rcf.

In some embodiments, step b) comprises heating, centrifuging, and filtering the paraffin-embedded sample. Heating, centrifuging, and filtering may be performed as described above.

Contacting an Embedded Sample with an Immiscible Solvent

In some embodiments, provided herein are methods involving contacting an embedded sample with an immiscible solvent in order to remove an embedding agent from an embedded sample, thereby generating a de-embedded sample. The embedding agent may be any one of the embedding agents described herein. The methods described herein may be used to separate embedding agents from embedded sample. In some embodiments, the methods described herein further comprise extracting an analyte from the de-embedded sample. The analyte may be any one of the analytes described herein.

In some embodiments, provided herein is a method of extracting an analyte, wherein the method comprises: providing an embedded sample comprising an analyte, wherein the sample is embedded in an embedding agent; removing the embedding agent from the embedded sample, thereby generating a de-embedded sample, wherein the embedding agent is removed by contacting the embedded sample with an immiscible solvent and separating the embedding agent from the sample; and extracting the analyte from the de-embedded sample. In some embodiments, the extraction of the analyte is improved relative to a method wherein the embedding agent is dissolved with a miscible solvent (e.g., xylene, ethyl acetate, CitriSolv™, or UltraClear™). In some embodiments, extraction of the analyte is improved relative to a method wherein the embedding agent is removed by cutting the embedding agent from the embedded sample.

In some embodiments, provided herein is a method of reducing the level of embedding agent in an analyte sample extracted from an embedded sample, wherein the method comprises: providing an embedded sample comprising an analyte, wherein the sample is embedded in an embedding agent; removing the embedding agent from the embedded sample, thereby generating a de-embedded sample, wherein the embedding agent is removed by contacting the embedded sample with an immiscible solvent and separating the embedding agent from the sample; extracting the analyte from the de-embedded sample; and purifying the extracted analyte to provide an analyte sample. In some embodiments, the level of embedding agent in the analyte sample is reduced relative to a method wherein the embedding agent is dissolved with a miscible solvent (e.g., xylene, ethyl acetate, CitriSolv™, or UltraClear™). In some embodiments, the level of embedding agent in the analyte sample is reduced relative to a method wherein the embedding agent is removed by cutting the embedding agent from the embedded sample. The level of embedding agent in the analyte sample may be measured, for example, by measuring the turbidity of the analyte sample. In some embodiments, the turbidity of the analyte sample is reduced relative to a method wherein the embedding agent is dissolved with a miscible solvent and/or relative to a method wherein the embedding agent is removed by cutting the embedding agent from the embedded sample.

In some embodiments, provided herein is a method of improving separation of embedding agent from an embedded sample, wherein the method comprises: providing an embedded sample comprising an analyte, wherein the sample is embedded in an embedding agent; removing the embedding agent from the embedded sample, thereby generating a de-embedded sample, wherein the embedding agent is removed by contacting the embedded sample with an immiscible solvent and separating the embedding agent from the sample; and extracting the analyte from the de-embedded sample. In some embodiments, the separation of embedding agent from the embedded sample is improved relative to a method wherein the embedding agent is dissolved with a miscible solvent (e.g., xylene, ethyl acetate, CitriSolv™, or UltraClear™). In some embodiments, the separation of embedding agent from the embedded sample is improved relative to a method wherein the embedding agent is removed by cutting the embedding agent from the embedded sample. In some embodiments, the method is performed in a liquid handling robot comprising a filter, wherein the improved separation of embedding agent results in less clogging of the filter relative to a method wherein the embedding agent is dissolved with a miscible solvent (e.g., xylene, ethyl acetate, CitriSolv™, or UltraClear™). In some embodiments, the method is performed in a liquid handling robot comprising a filter, wherein the improved separation of embedding agent results in less clogging of the filter relative to a method wherein the embedding agent is removed by cutting the embedding agent from the embedded sample. In some embodiments, the filter is a filter in a spin column. In some embodiments, the liquid handling robot is a Hamilton AutoLys STAR, and the spin column is an AutoLys tube.

In some embodiments, step b) does not comprise dissolving the embedding agent with a miscible solvent. In some embodiments, the miscible solvent is xylene, ethyl acetate, CitriSolv™ or UltraClear™.

Without wishing to be bound by theory, it is believed that any immiscible solvent which is less dense than water and denser than the embedding agent when the embedding agent is in a liquid form may be used in the methods described herein. For example, the density of paraffin density is approximately 0.8 g/cm³, the density of mineral oil is approximately 0.87 g/cm³, and the density of water is 1 g/cm³. Mineral oil is an example of a suitable immiscible solvent for separating water-based samples from paraffin. Without wishing to be bound by theory, it is believed that an immiscible solvent facilitates phase separation between an embedding agent (e.g., paraffin) and a sample (e.g., a lysate) by creating an intermedial layer. Accordingly, in some embodiments, the density of the immiscible solvent is lighter than water and heavier than the embedding agent when the embedding agent is in liquid form. In some embodiments, the density of the immiscible solvent is heavier than liquid paraffin. In some embodiments, the immiscible solvent is vegetable oil.

In some embodiments, the immiscible solvent is mineral oil. In some embodiments, the embedded sample is contacted with about 300, 350, 400, 450, 500, or 550 μL of mineral oil. In some embodiments, the embedded sample is contacted with mineral oil at a mineral oil to embedded sample ratio of about 1:4, 1:2, 1:1, 2:1, 4:1, 5:1, 6:1, 7:1, 8:1, 9:1, 10:1, 15:1, 20:1, 30:1, 40:1, or 50:1. In some embodiments, the mineral oil contacts the embedded sample at about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., or about 75° C. The method of any one of claims 218-221, wherein the mineral oil contacts the embedded sample for about 5 minutes, about 10 minutes, about 15 minutes, about 20 minutes, about 25 minutes, about 30 minutes, or about 35 minutes. In some embodiments, the mineral oil contacts the embedded sample while shaking at about 25 rpm, about 50 rpm, about 100 rpm, about 200 rpm, about 300 rpm, about 400 rpm, about 500 rpm, about 600 rpm, about 700 rpm, about 800 rpm, about 900 rpm, about 1000 rpm, or about 1100 rpm. In particular embodiments, the immiscible solvent is mineral oil and the embedding agent is paraffin.

In some embodiments, step b) comprises contacting the sample with an immiscible solvent, centrifuging, and filtering the embedded sample. In some embodiments, the embedded sample is centrifuged at about 1,700 rcf, about 1,750 rcf, about 1,800 rcf, about 1,850 rcf, or about 1,900 rcf. In some embodiments, the embedded sample is centrifuged at 1,811 rcf or greater. In some embodiments, the embedded sample is centrifuged at 1,811 rcf.

### Methods Involving Paraffin-Embedded Samples

In some embodiments, the embedding agent is paraffin. Accordingly, in some embodiments, provided herein are methods involving removing paraffin from paraffin-embedded samples. Exemplary methods involving removing paraffin from paraffin-embedded samples using methods involving contacting the paraffin-embedded sample with an immiscible solvent are described herein in the Examples.

In some embodiments, provided herein is a method of detecting alterations in RNA and/or DNA, wherein the method comprises: providing a paraffin-embedded sample; removing paraffin from the paraffin-embedded sample, thereby generating a deparaffinized sample, wherein the paraffin is removed by contacting the paraffin-embedded sample with an immiscible solvent and separating the paraffin from the sample; extracting RNA and/or DNA from the deparaffinized sample; and analyzing the RNA and/or DNA to detect alterations in the RNA and/or DNA. In some embodiments, the detection of alterations in the RNA and/or DNA is improved relative to a method wherein the paraffin is dissolved with a miscible solvent (e.g., xylene, ethyl acetate, CitriSolv™, or UltraClear™). In some embodiments, the detection of alterations in the RNA and/or DNA is improved relative to a method wherein the paraffin is removed by cutting the paraffin from the paraffin-embedded sample. In some embodiments, the detection of alterations in the RNA and/or DNA is improved relative to a method wherein the paraffin-embedded sample is not deparaffinized. In some embodiments, the alteration in the RNA and/or DNA is selected from the group consisting of: a copy number alteration; a point mutation; an in-frame deletion of one or more codons; an intragenic deletion; an intragenic insertion; a deletion of a full gene; an inversion; an interchromosomal translocation; a tandem duplication; a gene fusion; a genomic rearrangement that comprises an intron sequence; and/or a gene amplification or duplication. In some embodiments, the alteration in the RNA and/or the DNA is a copy number alteration. Methods of detecting alterations in RNA and/or DNA are described herein in detail below.

In some embodiments, provided herein is a method of extracting RNA and/or DNA, wherein the method comprises: providing a paraffin-embedded sample; removing paraffin from the paraffin-embedded sample, thereby generating a deparaffinized sample, wherein the paraffin is removed by contacting the paraffin-embedded sample with an immiscible solvent and separating the paraffin from the sample; and extracting RNA and/or DNA from the deparaffinized sample. In some embodiments, the extraction of the RNA and/or DNA is improved relative to a method wherein the paraffin is dissolved with a miscible solvent (e.g., xylene, ethyl acetate, CitriSolv™, or UltraClear™). In some embodiments, the extraction of the RNA and/or DNA is improved relative to a method wherein the paraffin is removed by cutting the paraffin from the paraffin-embedded sample. Methods of extracting RNA and/or DNA are described herein in detail below

In some embodiments, provided herein is a method of improving library construction for nucleic acid sequencing, wherein the method comprises: providing a paraffin-embedded sample; removing paraffin from the paraffin-embedded sample, thereby generating a deparaffinized sample, wherein the paraffin is removed by contacting the paraffin-embedded sample with an immiscible solvent and separating the paraffin from the sample; extracting RNA and/or DNA from the deparaffinized sample; and preparing a sequencing library for sequencing the RNA and/or the DNA. In some embodiments, the preparation of the sequencing library is improved relative to a method wherein the paraffin is dissolved with a miscible solvent (e.g., xylene, ethyl acetate, CitriSolv™, or UltraClear™). In some embodiments, the preparation of the sequencing library is improved relative to a method wherein the paraffin is removed by cutting the paraffin from the paraffin-embedded sample.

In some embodiments, provided herein is a method of reducing the level of paraffin in an RNA or DNA sample extracted from a paraffin-embedded sample, wherein the method comprises: providing a paraffin-embedded sample; removing paraffin from the paraffin-embedded sample, thereby generating a deparaffinized sample, wherein the paraffin is removed by contacting the paraffin-embedded sample with an immiscible solvent and separating the paraffin from the sample; extracting RNA and/or DNA from the deparaffinized sample; and purifying the extracted RNA and/or DNA to provide an RNA and/or DNA sample. In some embodiments, the level of paraffin in the RNA and/or DNA sample is reduced relative to a method wherein the paraffin is dissolved with a miscible solvent (e.g., xylene, ethyl acetate, CitriSolv™, or UltraClear™). In some embodiments, the level of paraffin in the RNA and/or DNA sample is reduced relative to a method wherein the paraffin is removed by cutting the paraffin from the paraffin-embedded sample. The level of paraffin in the RNA and/or DNA sample may be measured, for example, by measuring the turbidity of the RNA and/or DNA sample. In some embodiments, the turbidity of the RNA and/or DNA sample is reduced relative to a method wherein the paraffin is dissolved with a miscible solvent and/or relative to a method wherein the paraffin is removed by cutting the paraffin from the embedded sample. In some embodiments, the turbidity of an RNA and/or DNA sample prepared by a method wherein the paraffin is dissolved with a miscible solvent and/or a method wherein the paraffin is removed by cutting the paraffin from the paraffin-embedded sample is 2-fold, 3-fold, 4-fold, 5-fold, 6-fold, 7-fold, 8-fold, 9-fold, 10-fold, 20-fold, 30-fold, 40-fold, or 50-fold the turbidity of an RNA and/or DNA sample prepared using a method wherein the paraffin is removed by inducing a phase transition in the paraffin and separating the paraffin from the sample.

In some embodiments, provided herein is a method of improving separation of paraffin from a paraffin-embedded sample, wherein the method comprises: providing a paraffin-embedded sample; removing paraffin from the paraffin-embedded sample, thereby generating a deparaffinized sample, wherein the paraffin is removed by contacting the paraffin-embedded sample with an immiscible solvent and separating the paraffin from the sample; and extracting RNA and/or DNA from the deparaffinized sample. In some embodiments, the separation of paraffin from the paraffin-embedded sample is improved relative to a method wherein the paraffin is dissolved with a miscible solvent (e.g., xylene, ethyl acetate, CitriSolv™, or UltraClear™). In some embodiments, the separation of paraffin from the paraffin-embedded sample is improved relative to a method wherein the paraffin is removed by cutting the paraffin from the paraffin-embedded sample. In some embodiments, the method is performed in a liquid handling robot comprising a filter, wherein the improved separation of paraffin results in less clogging of the filter relative to a method wherein the paraffin is dissolved with a miscible solvent (e.g., xylene, ethyl acetate, CitriSolv™, or UltraClear™). As described above, the level of paraffin in the RNA and/or DNA sample may be measured, for example, by measuring the turbidity of the RNA and/or DNA sample. In some embodiments, a measurement of the turbidity of the RNA and/or DNA sample indicates that separation of the paraffin is improved relative to a method wherein the paraffin is dissolved with a miscible solvent and/or relative to a method wherein the paraffin is removed by cutting the paraffin from the paraffin-embedded sample. In some embodiments, the turbidity of an RNA and/or DNA sample prepared by a method wherein the paraffin is dissolved with a miscible solvent and/or a method wherein the paraffin is removed by cutting the paraffin from the paraffin-embedded sample is 2-fold, 3-fold, 4-fold, 5-fold, 6-fold, 7-fold, 8-fold, 9-fold, 10-fold, 20-fold, 30-fold, 40-fold, or 50-fold the turbidity of an RNA and/or DNA sample prepared using a method wherein the paraffin is removed by inducing a phase transition in the paraffin and separating the paraffin from the sample. In some embodiments, the method is performed in a liquid handling robot comprising a filter, wherein the improved separation of paraffin results in less clogging of the filter relative to a method wherein the paraffin is dissolved with a miscible solvent. In some embodiments, the miscible solvent is xylene, ethyl acetate, CitriSolv™, or UltraClear™. In some embodiments, the method is performed in a liquid handling robot comprising a filter, wherein the improved separation of paraffin results in less clogging of the filter relative to a method wherein the paraffin is removed by cutting the paraffin from the paraffin-embedded sample. In some embodiments, the filter is a filter in a spin column. In some embodiments, the liquid handling robot is a Hamilton AutoLys STAR, and the spin column is an AutoLys tube.

In some embodiments, step b) does not comprise dissolving the paraffin with a miscible solvent, optionally wherein the miscible solvent is xylene, ethyl acetate, CitriSolv™, or UltraClear™.

As described above, the density of paraffin density is approximately 0.8 g/cm³, the density of mineral oil is approximately 0.87 g/cm³, and the density of water is 1 g/cm³. Accordingly, it is believed that mineral oil is an example of a suitable immiscible solvent for separating water-based samples from paraffin. Without wishing to be bound by theory, it is believed that an immiscible solvent facilitates phase separation between an embedding agent (e.g., paraffin) and a sample (e.g., a lysate) by creating an intermedial layer. In some embodiments, the immiscible solvent is vegetable oil. In some embodiments, the immiscible solvent is mineral oil. In some embodiments, the paraffin-embedded sample is contacted with about 300, 350, 400, 450, 500, or 550 μL of mineral oil. In some embodiments, the paraffin-embedded sample is contacted with mineral oil at a mineral oil to paraffin-embedded sample ratio of about 1:4, 1:2, 1:1, 2:1, 4:1, 5:1, 6:1, 7:1, 8:1, 9:1, 10:1, 15:1, 20:1, 30:1, 40:1, or 50:1. In some embodiments, the mineral oil contacts the paraffin-embedded sample at about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., or about 75° C. In some embodiments, the mineral oil contacts the paraffin-embedded sample for about 5 minutes, about 10 minutes, about 15 minutes, about 20 minutes, about 25 minutes, about 30 minutes, or about 35 minutes. In some embodiments, the mineral oil contacts the paraffin-embedded sample while shaking at about 25 rpm, about 50 rpm, about 100 rpm, about 200 rpm, about 300 rpm, about 400 rpm, about 500 rpm, about 600 rpm, about 700 rpm, about 800 rpm, about 900 rpm, about 1000 rpm, or about 1100 rpm.

In some embodiments, step b) comprises contacting the sample with an immiscible solvent, centrifuging, and filtering the paraffin-embedded sample. In some embodiments, the paraffin-embedded sample is centrifuged at about 1,700 rcf, about 1,750 rcf, about 1,800 rcf, about 1,850 rcf, or about 1,900 rcf. In some embodiments, the paraffin-embedded sample is centrifuged at 1,811 rcf or greater. In some embodiments, the paraffin-embedded sample is centrifuged at 1,811 rcf.

In some embodiments, the separation of the paraffin from the sample is automated. In some embodiments, step b) is automated. In some embodiments, the method is automated. In some embodiments, two or more paraffin-embedded samples are processed in parallel. In some embodiments, 12, 24, 48, or 96 paraffin-embedded samples are processed in parallel.

In some embodiments, the method is performed using a liquid handling robot. In some embodiments, the liquid handling robot is an Agilent, BioTek, Hamilton, Tecan, or ThermoFisher Scientific liquid handling robot, optionally wherein the liquid handling robot is a Hamilton AutoLys STAR, a Hamilton STAR, a BioTek Dispenser, or a KingFisher Flex. In some embodiments, the liquid handling robot is a Hamilton AutoLys STAR.

In some embodiments, step c) comprises extracting RNA and DNA from the deparaffinized sample. Methods of extracting RNA and DNA are described in detail below.

### II. Embedded Samples

The methods described herein may be used to remove embedding agents from embedded samples. The embedding agent may be any one of the embedding agents described herein. In some embodiments, the embedding agent is selected from the group consisting of paraffin, resin, celloidin, Paraplast®, gelatin, ester wax, wax, polyethylene glycol, and nitrocellulose. In some embodiments, the embedding agent is Paraplast®. In some embodiments, the embedding agent is paraffin.

In some embodiments, the sample is a biological sample, such a sample comprising cells and/or tissues. In some embodiments, the sample is derived from an individual. In some embodiments, the sample is a mammalian sample. In some embodiments, the sample is a human sample.

### Paraffin-Embedded Samples

In some embodiments, provided herein are methods for removing paraffin from paraffin-embedded samples, thereby generating deparaffinized samples.

Various paraffin-embedded samples are known in the art and are suitable for use with the methods described herein. In some embodiments, the paraffin-embedded sample is from an individual known to have cancer or suspected of having cancer. In some embodiments, the individual is suspected of having any one of the cancers described herein. In some embodiments, the paraffin-embedded sample is from a biopsy, optionally a tumor biopsy.

In some embodiments, the paraffin-embedded sample is a fixed paraffin-embedded sample. In some embodiments, the fixed paraffin-embedded sample is selected from the group consisting of a formalin-fixed sample, an ethanol-fixed sample, and a methanol-fixed sample. In some embodiments, the paraffin-embedded sample is derived from a formalin-fixed paraffin-embedded (FFPE) tissue.

In some embodiments, the paraffin-embedded sample is derived from a cryopreserved tissue. In some embodiments, the paraffin-embedded sample is derived from a fresh-frozen tissue. In some embodiments, the fresh-frozen tissue is frozen in an optimal cutting temperature (OCT) compound.

In some embodiments, the paraffin-embedded sample is derived from a primary tissue obtained directly from a source of interest by any appropriate means. For example, in some embodiments, the paraffin-embedded sample is derived from a tissue is obtained by a method chosen from biopsy (e.g., fine needle aspiration or tissue biopsy) and surgery. In one embodiment, the paraffin-embedded sample is derived from a tissue comprising one or more cells associated with a tumor, e.g., tumor cells or tumor-infiltrating lymphocytes (TIL). In one embodiment, the paraffin-embedded sample is derived from a tissue including one or more premalignant or malignant cells. In one embodiment, the paraffin-embedded sample is derived from a tissue acquired from a hematologic malignancy (or pre-malignancy), e.g., a hematologic malignancy (or pre-malignancy) described herein. In one embodiment, the paraffin-embedded sample is derived from a tissue acquired from a cancer, such as a cancer described herein. In some embodiments, the paraffin-embedded sample is derived from a tissue acquired from a solid tumor, a soft tissue tumor or a metastatic lesion. In other embodiments, the paraffin-embedded sample is derived from tissue includes tissue or cells from a surgical margin. In some embodiments, the paraffin-embedded sample comprises tumor cells of interest. In some embodiments, the paraffin-embedded sample further comprises non-tumor cells. Provided herein are methods comprising extracting a paraffin-embedded sample from a tissue is from an individual suspected of having cancer. In some embodiments, the tissue comprises tumor cells of interest.

In some embodiments, the individual is suspected of having any one of the cancers described herein. In some embodiments, the tumor cells of interest are tumor cells associated with any one of the cancers described herein. In some embodiments, the cancer is acute lymphoblastic leukemia (“ALL”), acute myeloid leukemia (“AML”), adenocarcinoma, adenocarcinoma of the lung, adrenocortical cancer, adrenocortical carcinoma, anal cancer, appendiceal cancer, B-cell derived leukemia, B-cell derived lymphoma, B-cell lymphoma, bladder cancer, brain cancer, breast cancer (e.g., triple negative breast cancer (TNBC) or non-triple negative breast cancer), cancer of the fallopian tube(s), cancer of the testes, carcinoma, cerebral cancer, cervical cancer, cholangiocarcinoma, choriocarcinoma, chronic myelogenous leukemia, central nervous system (CNS) tumor, CNS cancer, colon cancer, colorectal cancer (e.g., colon adenocarcinoma), diffuse intrinsic pontine glioma (DIPG), diffuse large B cell lymphoma (“DLBCL”), embryonal rhabdomyosarcoma (ERMS), endometrial cancer, epithelial cancer, epithelial neoplasm, thymoma, esophageal cancer, Ewing's sarcoma, eye cancer (e.g., uveal melanoma), eyelid cancer, follicular lymphoma (“FL”), gall bladder cancer, gastric cancer, gastrointestinal cancer, glioblastoma, polycythemia vera, glioblastoma multiforme, glioma (e.g., lower grade glioma), gullet cancer, head and neck cancer, a hematological cancer, hepatocellular cancer, hepatocellular carcinoma, Hodgkin's lymphoma (HL), a heavy chain disease, intestinum rectum cancer, renal cancer, kidney cancer (e.g., kidney clear cell cancer, kidney chromophobe cancer, kidney clear cell cancer, kidney papillary cancer), large B-cell lymphoma, large intestine cancer, laryngeal cancer, leucosis, leukemia, liver cancer, lung cancer (e.g., lung adenocarcinoma, or non-small cell lung cancer), lymphoma, mammary gland cancer, melanoma (e.g., metastatic malignant melanoma), Hodgkin's disease, Waldenstrom's macroglobulinemia, Merkel cell carcinoma, mesothelioma, monocytic leukemia, multiple myeloma, myeloma, myogenic sarcoma, nasopharyngeal cancer, neuroblastic-derived CNS tumor (e.g., neuroblastoma (NB)), neuroma, astrocytoma, pilocytic astrocytoma, anaplastic astrocytoma, medulloblastoma, craniopharyngioma, ependymoma, pinealoma, hemangioblastoma, acoustic neuroma, oligodendroglioma, meningioma, vestibular schwannoma, adenoma, metastatic brain tumor, spinal tumor, non-Hodgkin's lymphoma (NHL), oral cancer, oral cavity cancer, osteosarcoma, ovarian cancer, ovarian carcinoma, pancreatic adenocarcinoma, pancreatic cancer, peritoneal cancer, pheochromocytoma, primary mediastinal B-cell lymphoma, primary peritoneal cancer, prostate cancer (e.g., hormone refractory prostate adenocarcinoma), rectal cancer (rectum carcinoma), relapsed or refractory classic Hodgkin's Lymphoma (cHL), salivary gland cancer (e.g., salivary gland tumor), skin cancer, small cell lung cancer, small intestine cancer, soft tissue sarcoma, squamous carcinoma, squamous cell carcinoma (e.g., squamous cell carcinoma of the anogenital region, squamous cell carcinoma of the anus, squamous cell carcinoma of the cervix, squamous cell carcinoma of the esophagus, squamous cell carcinoma of the head and neck (SCHNC), squamous cell carcinoma of the lung, squamous cell carcinoma of the penis, squamous cell carcinoma of the vagina, or squamous cell carcinoma of the vulva), stomach cancer, T-cell derived leukemia, T-cell lymphoma, testicular cancer, testicular tumor, thymic cancer, thyroid cancer (thyroid carcinoma), tongue cancer, tunica conjunctiva cancer, urinary bladder cancer, urothelial cell carcinoma, uterine cancer (e.g., uterine endometrial cancer or uterine sarcoma such as uterine carcinosarcoma), uterine endometrial cancer, uterus cancer, vaginal cancer, vulvar cancer, or Wilms' tumor.

In some embodiments, the cancer is a hematologic cancer (e.g., a hematologic malignancy), such as diffuse large B cell lymphoma (“DLBCL”), Hodgkin's lymphoma (“HL”), Non-Hodgkin's lymphoma (“NHL”), follicular lymphoma (“FL”), acute myeloid leukemia (“AML”), acute lymphoblastic leukemia (“ALL”), multiple myeloma (“MM”), acute lymphoblastic B-cell leukemia, acute lymphoblastic T-cell leukemia, acute myeloblastic leukemia, acute promyelocytic leukemia (“APL”), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (“CML”), chronic lymphocytic leukemia (“CLL”), or hairy cell leukemia. In some embodiments, a hematologic cancer of the disclosure is an acute or a chronic leukemia, such as a lymphoblastic, myelogenous, lymphocytic, or myelocytic leukemia. In some embodiments, a hematologic cancer of the disclosure is a lymphoma (e.g., Hodgkin's lymphoma, such as relapsed or refractory classic Hodgkin's Lymphoma (cHL), a non-Hodgkin's lymphoma, a diffuse large B-cell lymphoma, or a precursor T-lymphoblastic lymphoma), a lymphoepithelial carcinoma, or a malignant histiocytosis.

In some embodiments, the cancer is a solid tumor (e.g., a solid malignancy), such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, osteosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilms tumor, cervical cancer, uterine cancer, testicular cancer, non-small cell lung cancer (NSCLC), small cell lung carcinoma, bladder carcinoma, lung cancer, epithelial carcinoma, skin cancer, melanoma, neuroblastoma (NB), or retinoblastoma.

In certain embodiments, the cancer is a cancer of the adrenal glands (such as neuroblastoma), bladder cancer (such as urothelial (transitional cell) carcinoma), brain cancer (such as anaplastic astrocytoma or glioblastoma), bone cancer (such as osteosarcoma), bone marrow cancer (such as B-cell acute leukemia (B-ALL) or multiple myeloma), breast cancer (such as invasive ductal carcinoma), head and neck cancer (such as adenocarcinoma, mucoepidermoid carcinoma, squamous cell carcinoma), lymph node cancer, lung cancer (e.g., mucoepidermoid carcinoma, sarcoma, small cell undifferentiated carcinoma, adenocarcinoma, adenosquamous carcinoma, large cell carcinoma, large cell neuroendocrine carcinoma, non-small cell lung carcinoma, non-small cell lung carcinoma not otherwise specified, or squamous cell carcinoma), female reproductive cancer (e.g., cancer of the fallopian tubes such as fallopian tube serous carcinoma; ovarian cancer, such as epithelial carcinoma, epithelial carcinoma not otherwise specified, high grade serous carcinoma, low grade serous carcinoma, serous carcinoma; and uterine cancer, such as carcinosarcoma, endometrial adenocarcinoma, endometrial adenocarcinoma not otherwise specified, papillary serous endometrial adenocarcinoma, leiomyosarcoma, sarcoma, sarcoma not otherwise specified, or smooth muscle tumor of uncertain malignant potential (STUMP)), gallbladder cancer (such as adenocarcinoma), cancer of the gastroesophageal junction (such as adenocarcinoma), lymph node cancer (such as anaplastic large cell lymphoma, B-cell lymphoma, B-cell lymphoma not otherwise specified, diffuse large B cell lymphoma, non-Hodgkin's lymphoma, non-Hodgkin's lymphoma not otherwise specified), colon cancer (such as adenocarcinoma), colorectal cancer, skin cancer (such as melanoma or squamous cell carcinoma), small intestine cancer (adenocarcinoma), soft tissue cancer (such as Ewing sarcoma, fibrosarcoma, histiocytosis, histiocytosis not otherwise specified, juvenile xanthogranuloma or non-Langerhans cell histiocytosis, inflammatory myofibroblastic tumor, leiomyosarcoma, neurofibroma, neuroblastoma, sarcoma not otherwise specified, sarcoma, undifferentiated sarcoma, or an undifferentiated soft tissue cancer), pancreatic cancer (such as carcinoma, carcinoma not otherwise specified, ductal adenocarcinoma, or mucinous cystadenocarcinoma), prostate cancer (such as acinar adenocarcinoma), pericardium cancer (such as mesothelioma), peritoneum cancer (such as mesothelioma), salivary gland cancer (such as carcinoma or carcinoma not otherwise specified), stomach cancer (such as adenocarcinoma, adenocarcinoma not otherwise specified, or diffuse type cancer), kidney cancer (such as renal cell carcinoma or renal cell carcinoma not otherwise specified), thyroid cancer (such as carcinoma, carcinoma not otherwise specified, or papillary carcinoma), or a cancer of unknown primary origin (such as adenocarcinoma, carcinoma, carcinoma not otherwise specified, leiomyosarcoma, malignant neoplasm, malignant neoplasm not otherwise specified, melanoma, myoepithelial carcinoma, squamous cell carcinoma (SCC), or undifferentiated neuroendocrine carcinoma).

In some embodiments, the cancer is a cancer that is recurrent or refractory to one or more prior anti-cancer therapies.

In some embodiments, the cancer is any cancer type provided in Ross et al., Oncologist (2017) 22(12):1444-1450, which is incorporated herein by reference.

### Methods Involving Precision Enrichment of Paraffin-Embedded Samples

In some embodiments, the methods described herein may be performed using samples derived from precision enrichment-based methods. Methods involving precision enrichment are described, for example, in U.S. Provisional Application No. 63/189,602, which is hereby incorporated by reference in its entirety. In some embodiments of the methods described herein, step a) comprises: i) identifying a target region comprising tumor cells of interest in a paraffin-embedded tissue; ii) extracting the paraffin-embedded sample from the paraffin-embedded tissue; iii) identifying the location of the paraffin-embedded sample in the paraffin-embedded tissue; and iv) if the location of the paraffin-embedded sample overlaps with the target region comprising tumor cells of interest, extracting RNA and/or DNA from the sample. In some embodiments, if the location of the paraffin-embedded sample does not overlap with the target region comprising tumor cells of interest, steps ii) and iii) are repeated.

This aspect present disclosure is based, at least in part, on the development of methods for extracting nucleic acids from tissues that comprise tumor cells of interest, such as formalin-fixed paraffin-embedded (FFPE) tissues. The tissues may be from a subject suspected of having cancer, or known to have cancer. Without wishing to be bound by any particular theory, it is believed that the inclusion of a step of analyzing the tissue after extracting the sample is informative with respect to whether the sample has successfully enriched for the tumor cells of interest, or whether a further sample should be extracted from the tissue. For example, a slide of the tissue may be prepared after the sample is extracted in order to determine the degree of overlap with the sample and the tumor cells of interest. Such a histologic quality assurance/quality control step is believed to allow for the assessment of the tumor content enrichment process. Without such a quality assurance/quality control step, samples extracted from tissues have occasionally failed during sequencing analysis due to low tumor purity. These samples were not usable, and were considered to be unusable due to a “likely missed enrichment.” Accordingly, the methods described herein enable testing of specimens that otherwise would produce a level of tissue that was insufficient for analysis of tumor content with prior enrichment methods, and reduce the occurrence of unusable samples due to likely missed enrichments. The successful enrichment of tumor content is of particular importance for the assessment of certain biomarkers that may indicate the presence of cancer and require a relatively high level of tumor content in order to measure the biomarkers. Accordingly, the methods described herein may be used to detect the presence of biomarkers that may have otherwise been undetectable. This is thought to improve the specificity and precision of subsequent sequence analyses of the biomarkers. The methods described herein may be referred to as “precision enrichment” methods, as they involve the precision enrichment of tumor cells of interest, and therefore tumor content, and nucleic acids derived from tumor cells. Methods involving precision enrichment may be used in combination with any of the methods described above.

In some embodiments, the methods described herein comprise extracting a paraffin-embedded sample from a paraffin-embedded tissue using a needle. In some embodiments, the needle is punched through the paraffin-embedded tissue, thereby extracting the sample. In some embodiments, the needle is a disposable needle. In some embodiments, the needle is a thin-walled needle. In some embodiments, the needle is a blunt tipped needle. In some embodiments, the needle is a stainless steel needle. In some embodiments, the needle is a hypodermic needle. In some embodiments, the needle comprises a Luer-compatible hub. In some embodiments, the needle is a 13 gauge needle, 14 gauge needle, a 15 gauge needle, a 16 gauge needle, a 17 gauge needle, a 18 gauge needle, a 19 gauge needle, a 20 gauge needle, or a 21 gauge needle. In some embodiments, the paraffin-embedded sample extracted from the paraffin-embedded tissue is about 0.8, 0.9, 1.0, 1.2, 1.4, 1.6, 1.8, 2.0, or 2.3 mm in diameter

In some embodiments, step b) comprises extracting the paraffin-embedded sample using laser microdissection (LMD). In some embodiments, step b) comprises extracting the paraffin-embedded sample using a razor blade.

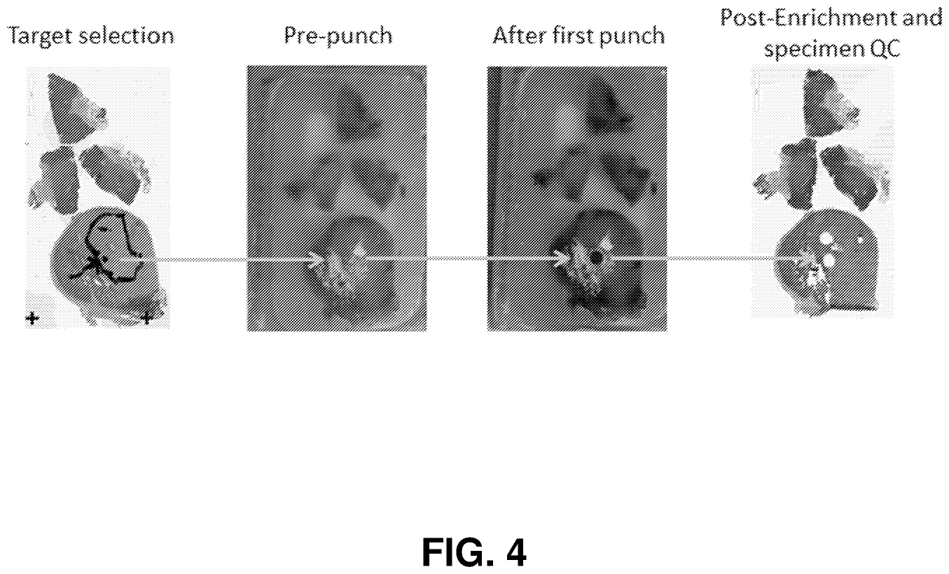

In some embodiments, step c) of the methods described herein comprises preparing a slide of a section of the paraffin-embedded tissue. In general, the preparation of a slide of a section of the paraffin-embedded tissue is thought to allow for the assessment of whether the paraffin-embedded sample (e.g., a paraffin-embedded sample extracted with a needle as described above), has successfully enriched for the tumor cells of interest. For example, the slide of a section of the paraffin-embedded tissue may reveal that the position of the paraffin-embedded sample (e.g., the position of the needle punch where the sample was extracted) overlaps with the position of the tumor cells of interest. Exemplary images of slides of sections of paraffin-embedded tissues are provided in FIG. 4. As shown in FIG. 4, the position of a needle punch is visible in the section of the paraffin-embedded tissue. In some embodiments, the section of the paraffin-embedded tissue is stained. In some embodiments, the section of the paraffin-embedded tissue is stained in such a way that distinguishes the tumor cells of interest from other cells in the paraffin-embedded tissue. In some embodiments, the section of the paraffin-embedded tissue is Haematoxylin and Eosin (H&E) stained. In some embodiments, the section of the paraffin-embedded tissue is immunostained, e.g., using a detectably-labeled antibody. In some embodiments, the detectably-labeled antibody binds to a protein expressed in the tumor cells of interest. In some embodiments, step c) is performed by visual inspection. For example, in some embodiments, step c) is performed by a pathologist who visually inspects a slide of a section of the paraffin-embedded tissue and determines whether the location of the paraffin-embedded sample overlaps with the tumor cells of interest. In some embodiments, step c) is performed by a computer system. For example, in some embodiments, step c) is performed using a computer system that assesses whether the location of the paraffin-embedded sample overlaps with the tumor cells of interest. In some embodiments, step c) is performed using an image analysis system.

In some embodiments, the level of enrichment of tumor cells of interest in the paraffin-embedded sample is 95%, 90%, 85%, 80%, 75%, 70%, 65%, 60%, 55%, 50%, or higher. In some embodiments, the level of enrichment of tumor cells of interest in the paraffin-embedded sample is at least 1-fold, 1.1-fold, 1.2-fold, 1.3-fold, 1.4-fold, 1.5-fold, 1.6-fold, 1.7-fold, 1.8-fold, 1.9-fold, or 2-fold higher than the level of tumor cells of interest in the remaining paraffin-embedded tissue. In some embodiments, the paraffin-embedded sample comprises cells comprising least 25%, at least 30%, at least 35%, at least 40%, at least 45%, at least 50%, at least 55%, at least 60%, at least 65%, at least 70%, at least 75%, at least 80%, at least 85%, or at least 90% or more tumor cells of interest.

In some embodiments, step ii) further comprises inspecting the paraffin-embedded sample, and optionally removing excess tissue from the paraffin-embedded sample.

III. Extraction of Analytes from De-Embedded Samples

The methods herein involve the extraction of analytes from de-embedded samples. In some embodiments in which the de-embedded sample is a biological sample, the analyte may be any macromolecule or small molecule that may be extracted from the biological sample. In some embodiments, the analyte is selected from the group consisting of a polypeptide, RNA, DNA, a small molecule, a lipid, a polysaccharide, an exosome, a mitochondria, and a nucleus. In some embodiments, the analyte is an organelle. In some embodiments, two or more analytes are extracted from the de-embedded sample, such as 2, 3, 4, 5, 6, 7, 8, 9, or 10 analytes.

In some embodiments, the analyte is one or more nucleic acids. In some embodiments, the one or more nucleic acids comprise RNA and/or DNA. In some embodiments, the one or more nucleic acids comprise genomic DNA, cDNA, or mRNA. In some embodiments, the analyte is RNA. In some embodiments, the analyte is DNA.

Extraction of RNA and/or DNA from Deparaffinized Samples

### RNA-First Extraction Methods

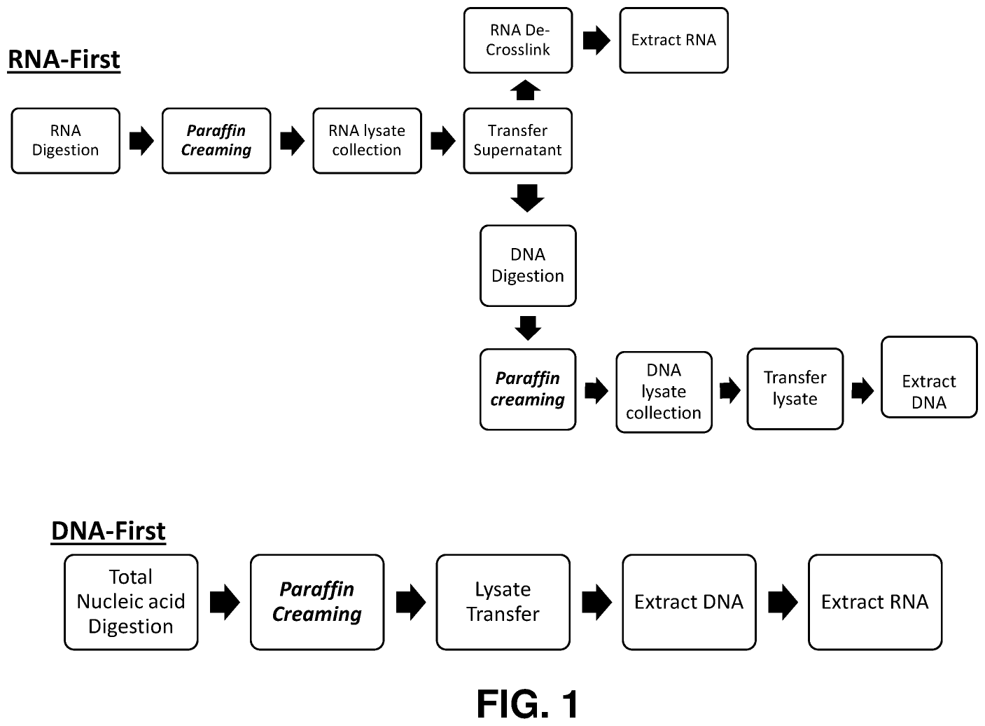

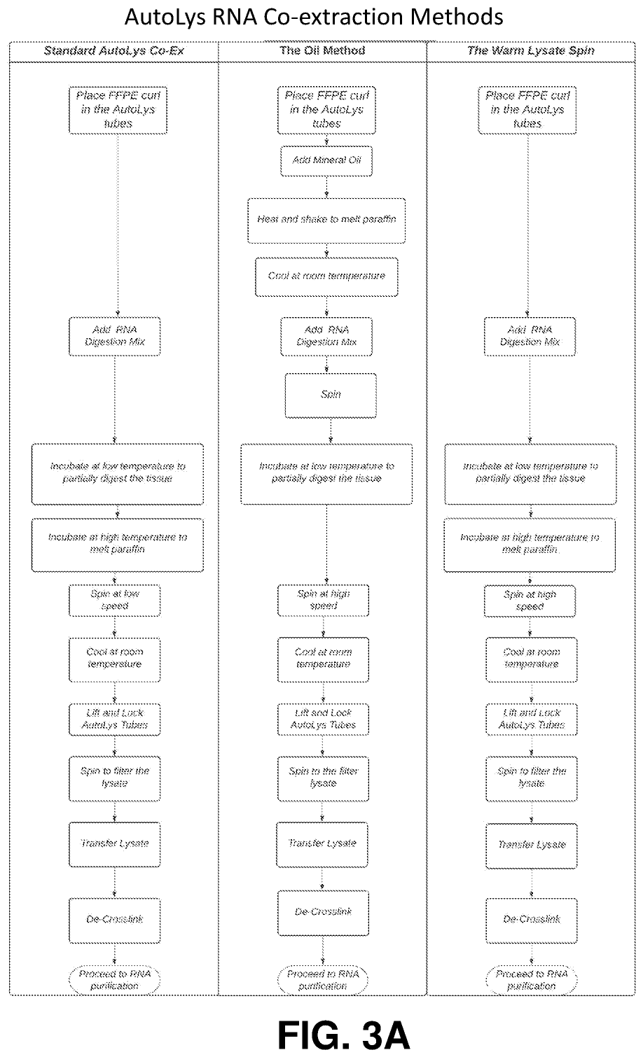

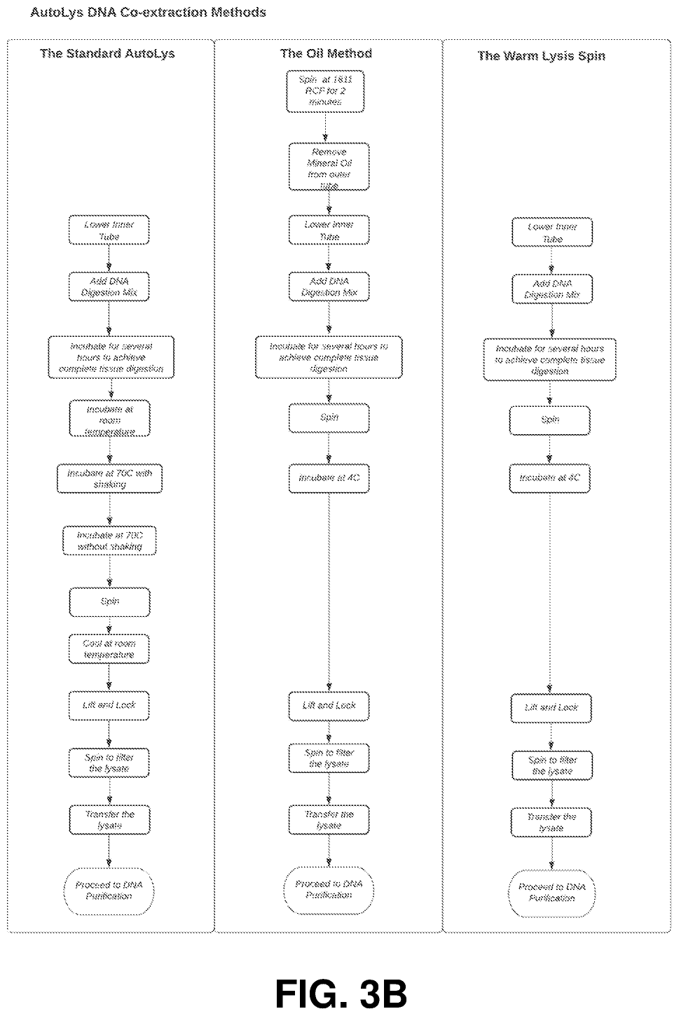

In some embodiments, the methods described herein involve extracting RNA and/or DNA from de-paraffinized samples. In particular embodiments, the RNA is extracted before the DNA is extracted. Exemplary methods in which RNA is extracted before DNA is extracted are described in the Examples, and diagrammed in FIG. 1 and FIG. 3A.

In some embodiments, the method in which the RNA is extracted before the DNA further comprises digesting the paraffin-embedded sample before step b). In some embodiments, the paraffin-embedded sample is digested using a proteinase. In some embodiments, the proteinase is proteinase K. In some embodiments, the paraffin-embedded sample is digested using about 10 μL, about 15 μL, about 20 μL, about 25 μL, about 30 μL, or about 35 μL of 20 mg/mL proteinase K. In some embodiments, the paraffin-embedded sample is incubated with the proteinase at about 50° C., about 51° C., about 52° C., about 53° C., about 54° C., about 55° C., about 56° C., about 57° C., about 58° C., about 59° C., about 60° C., about 61° C., or about 62° C. In some embodiments, the paraffin-embedded sample is incubated with the proteinase for about 10 minutes, about 11 minutes, about 12 minutes, about 13 minutes, about 14 minutes, about 15 minutes, about 16 minutes, about 17 minutes, about 18 minutes, about 19 minutes, or about 20 minutes. In some embodiments, the paraffin-embedded sample is incubated with the proteinase shaking at about 500 rpm, 600 rpm, 700 rpm, 800 rpm, 900 rpm, 1000 rpm, 1100 rpm, 1200 rpm, 1300 rpm, 1400 rpm, or 1500 rpm. In some embodiments, the paraffin-embedded sample is partially digested or completely digested.

In some embodiments, the method in which the RNA is extracted before the DNA further comprises de-crosslinking the digested sample after step b). In some embodiments, de-crosslinking comprises heating the digested sample to 80-90° C. In some embodiments, the digested sample is heated for about 10 minutes, about 11 minutes, about 12 minutes, about 13 minutes, about 14 minutes, about 15 minutes, about 16 minutes, about 17 minutes, about 18 minutes, about 19 minutes, or about 20 minutes in order to de-crosslink the digested sample.

In some embodiments, step c) comprises collecting a sample lysate comprising RNA from the digested paraffin-embedded sample, and purifying the RNA from the sample lysate comprising RNA. Methods of purifying RNA from a sample lysate comprising RNA are known in the art.

In some embodiments, the method in which the RNA is extracted before the DNA further comprises completely digesting the digested paraffin-embedded sample. In some embodiments, the complete digestion is performed using a proteinase. In some embodiments, the proteinase is proteinase K. In some embodiments, the digested paraffin-embedded sample is digested using about 10 μL, about 15 μL, about 20 μL, about 25 μL, about 30 μL, or about 35 μL of 20 mg/mL proteinase K. In some embodiments, the digested paraffin-embedded sample is incubated with the proteinase at about 65° C., about 66° C., about 67° C., about 68° C., about 69° C., about 70° C., about 71° C., about 72° C., about 73° C., about 74° C., or about 75° C. In some embodiments, the digested paraffin-embedded sample is incubated with the proteinase for about 6 hours, about 7 hours, about 8 hours, about 9 hours, about 10 hours, about 11 hours, 12 hours, about 13 hours, about 14 hours, about 15 hours, about 16 hours, about 17 hours, about 18 hours, about 19 hours, about 20 hours, about 21 hours, or about 22 hours. In some embodiments, the digested paraffin-embedded sample is incubated with the proteinase shaking at about 500 rpm, 600 rpm, 700 rpm, 800 rpm, 900 rpm, 1000 rpm, 1100 rpm, 1200 rpm, 1300 rpm, 1400 rpm, or 1500 rpm.

In some embodiments, the method in which the RNA is extracted before the DNA further comprises collecting a sample lysate comprising DNA from the completely digested paraffin-embedded sample. In some embodiments, the method further comprises purifying the DNA from the sample lysate comprising DNA. Methods of purifying DNA from a sample lysate comprising DNA are known in the art.

The methods described herein in which RNA is extracted before DNA are amenable to automation and/or high-throughput analyses. In some embodiments, the methods described herein in which RNA is extracted before DNA are performed using a liquid handling robot. In some embodiments, the liquid handling robot is an Agilent, BioTek, Hamilton, Tecan, or ThermoFisher Scientific liquid handling robot, optionally wherein the liquid handling robot is a Hamilton AutoLys STAR, a Hamilton STAR, a BioTek Dispenser, or a KingFisher Flex. In some embodiments, the liquid handling robot is a Hamilton AutoLys STAR. In some embodiments, the extraction of RNA before DNA is carried out for two or more samples processed in parallel. In some embodiments, 2, 4, 8, 16, 24, 48, 96 or more samples are processed in parallel.

### DNA-First Extraction Methods

In some embodiments, the methods described herein involve extracting RNA and/or DNA from de-paraffinized samples. In particular embodiments, the DNA is extracted before the RNA is extracted. Exemplary methods in which RNA is extracted before DNA is extracted are described in the Examples, and diagrammed in FIG. 1 and FIG. 3B.

In some embodiments, the method in which the DNA is extracted before the RNA further comprises completely digesting the paraffin-embedded sample after step b). In some embodiments, the complete digestion is performed using a proteinase. In some embodiments, the proteinase is proteinase K. In some embodiments, the paraffin-embedded sample is digested using about 10 μL, about 15 μL, about 20 μL, about 25 μL, about 30 μL, or about 35 μL of 20 mg/mL proteinase K. The method of any one of claims 125-127, wherein the paraffin-embedded sample is incubated with the proteinase at about 65° C., about 66° C., about 67° C., about 68° C., about 69° C., about 70° C., about 71° C., about 72° C., about 73° C., about 74° C., or about 75° C. In some embodiments, the paraffin-embedded sample is incubated with the proteinase for about 6 hours, about 7 hours, about 8 hours, about 9 hours, about 10 hours, about 11 hours, 12 hours, about 13 hours, about 14 hours, about 15 hours, about 16 hours, about 17 hours, about 18 hours, about 19 hours, about 20 hours, about 21 hours, or about 22 hours.

In some embodiments, the method in which the DNA is extracted before the RNA further comprises extracting the DNA from the completely digested paraffin-embedded sample. In some embodiments, the method further comprises purifying the extracted DNA. Methods of purifying extracted DNA are known in the art.

In some embodiments, the method in which the DNA is extracted before the RNA further comprises extracting the RNA from the completely digested paraffin-embedded sample. In some embodiments, the method comprises purifying the extracted RNA. Methods of purifying RNA from extracted RNA are known in the art.

The methods described herein in which DNA is extracted before RNA are amenable to automation and/or high-throughput analyses. In some embodiments, the methods described herein in which DNA is extracted before RNA are performed using a liquid handling robot. In some embodiments, the liquid handling robot is an Agilent, BioTek, Hamilton, Tecan, or ThermoFisher Scientific liquid handling robot, optionally wherein the liquid handling robot is a Hamilton AutoLys STAR, a Hamilton STAR, a BioTek Dispenser, or a KingFisher Flex. In some embodiments, the liquid handling robot is a Hamilton AutoLys STAR. In some embodiments, the extraction of DNA before RNA is carried out for two or more samples processed in parallel. In some embodiments, 2, 4, 8, 16, 24, 48, 96 or more samples are processed in parallel.

In some embodiments, regardless of whether the DNA is extracted before the RNA or the RNA is extracted before the DNA, the method further comprises measuring the amount of RNA and/or DNA extracted from the paraffin-embedded sample. In some embodiments, the amount of RNA and/or DNA extracted from the paraffin-embedded sample is about 5 ng, about 10 ng, about 20 ng, about 30 ng, about 40 ng, about 50 ng, about 60 ng, about 70 ng, about 80 ng, about 100 ng, about 50 μg, or about 50 mg.

### IV. Analysis of Analytes Extracted From De-Embedded Sample

The analytes extracted from de-embedded samples, as described above, may be further analyzed. Suitable methods for analyzing the analytes described herein are known in the art.

Analysis of RNA and/or DNA

The method of any one of the preceding claims, wherein the method further comprises analyzing the DNA extracted from the embedded sample to detect a somatic mutation selected from the group consisting of: i) a point mutation; ii) an in-frame deletion of one or more codons; iii) an intragenic deletion; iv) an intragenic insertion; v) a deletion of a full gene; vi) an inversion; vii) an interchromosomal translocation; viii) a tandem duplication; ix) a gene fusion; x) a genomic rearrangement that comprises an intron sequence; and/or xi) a gene amplification or duplication.

In some embodiments, the somatic mutation is in a gene, and wherein the gene is ABL1, AKT1, AKT2, AKT3, ALK, APC, AR, BRAF, CCND1, CDK4, CDKN2A, CEBPA, CTNNB1, EGFR, ERBB2, ESR1, FGFR1, FGFR2, FGFR3, FLT3, HRAS, JAK2, KIT, KRAS, MAP2K1, MAP2K2, MET, MLL, MYC, NF1, NOTCH1, NPM1, NRAS, NTRK3, PDGFRA, PIK3CA, PIK3CG, PIK3R1, PTCH1, PTCH2, PTEN, RB1, RET, SMO, STK11, SUFU, or TP53. In some embodiments, the somatic mutation is in a gene, and wherein the gene is ABL2, ARAF, ARFRP1, ARID1A, ATM, ATR, AURKA, AURKB, BAP1, BCL2, BCL2A1, BCL2L1, BCL2L2, BCL6, BRCA1, BRCA2, CBL, CARD11, CBL, CCND2, CCND3, CCNE1, CD79A, CD79B, CDH1, CDH2, CDH20, CDH5, CDK6, CDK8, CDKN2B, CDKN2C, CHEK1, CHEK2, CRKL, CRLF2, DNMT3A, DOT1L, EPHA3, EPHA5, EPHA6, EPHA7, EPHB1, EPHB4, EPHB6, ERBB3, ERBB4, ERG, ETV1, ETV4, ETV5, ETV6, EWSR1, EZH2, FANCA, FBXW7, FGFR4, FLT1, FLT4, FOXP4, GATA1, GNA11, GNAQ, GNAS, GPR124, GUCY1A2, HOXA3, HSP90AA1, IDH1, IDH2, IGF1R, IGF2R, IKBKE, IKZF1, INHBA, IRS2, JAK1, JAK3, JUN, KDM6A, KDR, LRP1B, LRP6, LTK, MAP2K4, MCL1, MDM2, MDM4, MEN1, MITF, MLH1, MPL, MRE11A, MSH2, MSH6, MTOR, MUTYH, MYCL1, MYCN, NF2, NKX2-1, NTRK1, NTRK2, PAK3, PAX5, PDGFRB, PKHD1, PLCG1, PRKDC, PTPN11, PTPRD, RAF1, RARA, RICTOR, RPTOR, RUNX1, SMAD2, SMAD3, SMAD4, SMARCA4, SMARCB1, SOX10, SOX2, SRC, TBX22, TET2, TGFBR2, TMPRSS2, TNFAIP3, TNK, TNKS2, TOP1, TSC1, TSC2, USP9X, VHL, or WT1.

In some embodiments, the method further comprises analyzing the RNA extracted from the embedded sample to detect an alternation selected from the group consisting of: i) gene fusions; ii) exon-skipping events; iii) splice variants; and/or iv) altered gene expression.

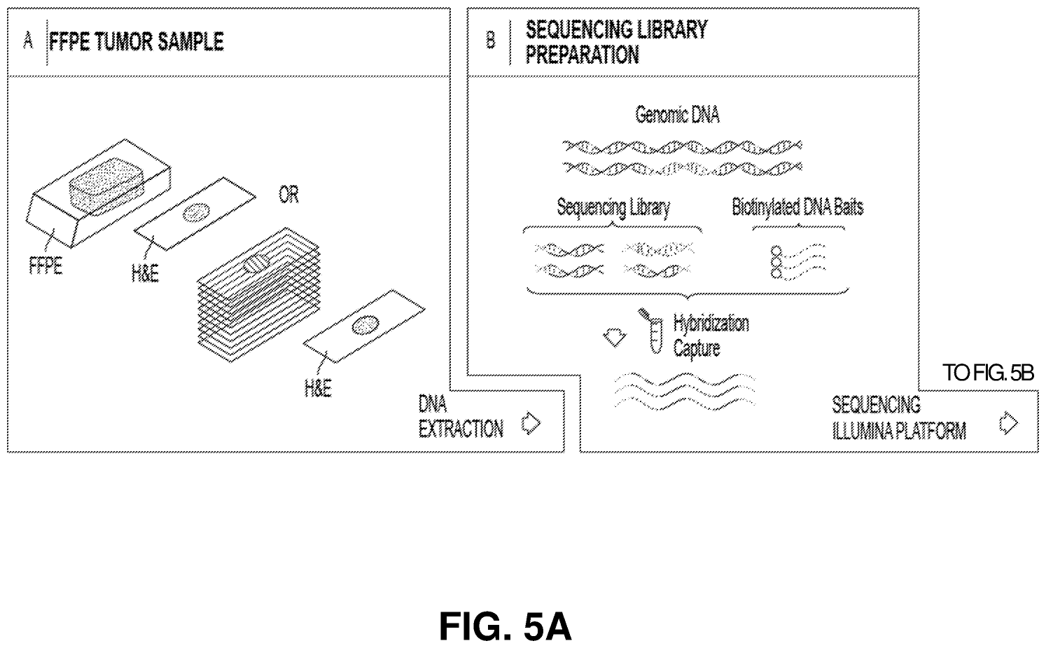

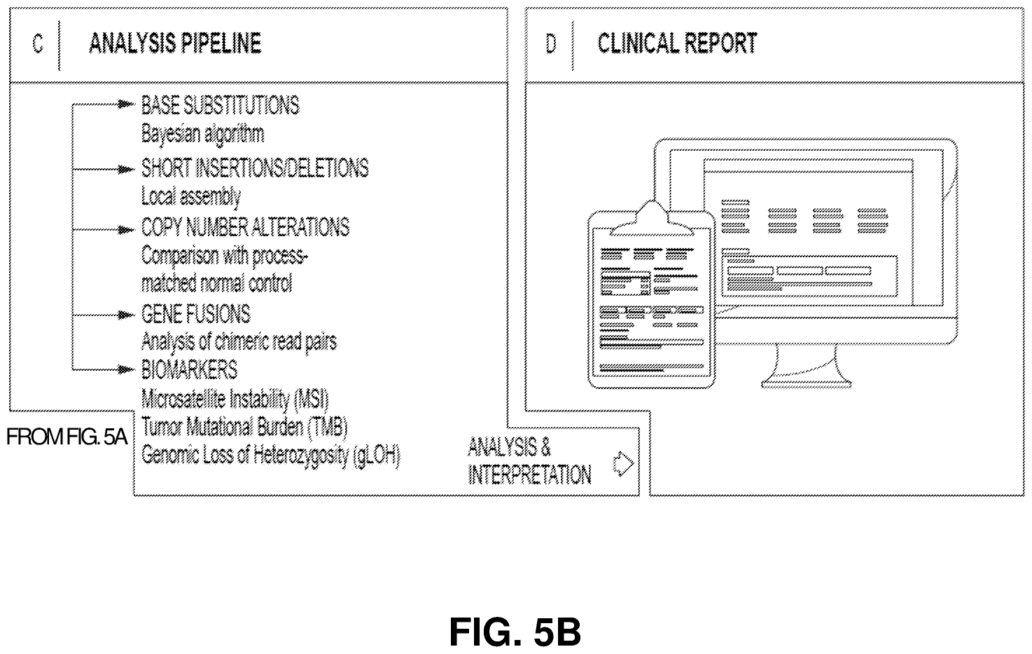

In some embodiments, the RNA and/or DNA extracted from the de-embedded sample are analyzed by one or more methods selected from the group consisting of a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), and mass-spectrometric genotyping. In some embodiments, the RNA and/or DNA extracted from the de-embedded sample are analyzed by next-generation sequencing. An exemplary method of next-generation sequencing is described in, for example, Frampton, G. M. et al. (2013) Nat. Biotech. 31:1023-1031. In some embodiments, the RNA and/or DNA extracted from the de-embedded sample are analyzed according to a method as diagrammed in FIGS. 5A-5B.

In some embodiments, the method further comprises: e) optionally, ligating one or more adaptors onto the RNA and/or DNA extracted from the de-embedded sample, thereby generating ligated nucleic acids; d) optionally, amplifying nucleic acids from the ligated nucleic acids; f) optionally, capturing a plurality of nucleic acids corresponding to a gene of interest; g) sequencing, by a sequencer, the plurality of nucleic acids to obtain a plurality of sequence reads corresponding to the gene of interest; h) analyzing the plurality of sequence reads; and i) based on the analysis, detecting one or more mutations in a gene of interest. In some embodiments, the plurality of nucleic acids corresponding to the gene of interest is captured from the amplified nucleic acids by hybridization with a bait molecule.

In some embodiments, a biomarker nucleic acid molecule of the disclosure is detected using any suitable method known in the art, such as a nucleic acid hybridization assay, an amplification-based assay (e.g., polymerase chain reaction, PCR), a PCR-RFLP assay, real-time PCR, sequencing (e.g., Sanger sequencing or next-generation sequencing), a screening analysis (e.g., using karyotype methods), fluorescence in situ hybridization (FISH), break away FISH, spectral karyotyping, multiplex-FISH, comparative genomic hybridization, in situ hybridization, single specific primer-polymerase chain reaction (SSP-PCR), high performance liquid chromatography (HPLC), or mass-spectrometric genotyping. Methods of analyzing samples, e.g., to detect a nucleic acid molecule, are described in U.S. Pat. No. 9,340,830 and in WO2012092426A1, which are hereby incorporated by reference in their entirety.

### In Situ Hybridization Methods

In some embodiments, a biomarker nucleic acid molecule of the disclosure is detected in the RNA and/or DNA extracted from the de-embedded sample using an in situ hybridization method, such as a fluorescence in situ hybridization (FISH) method.

In some embodiments, FISH analysis is used to identify the chromosomal rearrangement resulting in the mutations as described herein. In some embodiments, FISH analysis is used to identify an RNA molecule comprising a biomarker nucleic acid described herein. Methods for performing FISH are known in the art and can be used in nearly any type of tissue. In FISH analysis, nucleic acid probes which are detectably labeled, e.g. fluorescently labeled, are allowed to bind to specific regions of DNA, e.g., a chromosome, or an RNA, e.g., an mRNA, and then examined, e.g., through a microscope. See, for example, U.S. Pat. No. 5,776,688. DNA or RNA molecules are first fixed onto a slide, the labeled probe is then hybridized to the DNA or RNA molecules, and then visualization is achieved, e.g., using enzyme-linked label-based detection methods known in the art. Generally, the resolution of FISH analysis is on the order of detection of 60 to 100000 nucleotides, e.g., 60 base pairs (bp) up to 100 kilobase pairs of DNA. Nucleic acid probes used in FISH analysis comprise single stranded nucleic acids. Such probes are typically at least about 50 nucleotides in length. In some embodiments, probes comprise about 100 to about 500 nucleotides. Probes that hybridize with centromeric DNA and locus-specific DNA or RNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA or other sources of nucleic acids through standard techniques. Examples of probes, labeling and hybridization methods are known in the art.

Several variations of FISH methods are known in the art and are suitable for use according to the methods of the disclosure, including single-molecule RNA FISH, Fiber FISH, Q-FISH, Flow-FISH, MA-FISH, break-away FISH, hybrid fusion-FISH, and multi-fluor FISH or mFISH. In some embodiments, “break-away FISH” is used in the methods provided herein. In break-away FISH, at least one probe targeting a fusion junction or breakpoint and at least one probe targeting an individual gene of the fusion, e.g., at one or more exons and or introns of the gene, are utilized. In normal cells (i.e., cells not having a fusion nucleic acid molecule described herein), both probes are observed (or a secondary color is observed due to the close proximity of the two genes of the gene fusion); and in cells having a fusion nucleic acid molecule described herein, only a single gene probe is observed due to the presence of a rearrangement resulting in the fusion nucleic acid molecule.

### Array-Based Methods

In some embodiments, a biomarker nucleic acid molecule of the disclosure is detected in the RNA and/or DNA extracted from the de-embedded sample using an array-based method, such as array-based comparative genomic hybridization (CGH) methods. In array-based CGH methods, a first sample of nucleic acids (e.g., from a sample, such as from a tumor) is labeled with a first label, while a second sample of nucleic acids (e.g., a control, such as from a healthy cell/tissue) is labeled with a second label. In some embodiments, equal quantities of the two samples are mixed and co-hybridized to a DNA microarray of several thousand evenly spaced cloned DNA fragments or oligonucleotides, which have been spotted in triplicate on the array. After hybridization, digital imaging systems are used to capture and quantify the relative fluorescence intensities of each of the hybridized fluorophores. The resulting ratio of the fluorescence intensities is proportional to the ratio of the copy numbers of DNA sequences in the two samples. In some embodiments, where there are chromosomal deletions or multiplications, differences in the ratio of the signals from the two labels are detected and the ratio provides a measure of the copy number. Array-based CGH can also be performed with single-color labeling. In single color CGH, a control (e.g., control nucleic acid sample, such as from a healthy cell/tissue) is labeled and hybridized to one array and absolute signals are read, and a test sample (e.g., a nucleic acid sample obtained from an individual or from a tumor) is labeled and hybridized to a second array (with identical content) and absolute signals are read. Copy number differences are calculated based on absolute signals from the two arrays.

### Amplification-Based Methods

In some embodiments, a biomarker nucleic acid molecule of the disclosure is detected in the RNA and/or DNA extracted from the de-embedded sample using an amplification-based method. As is known in the art, in such amplification-based methods, a sample of nucleic acids, such as a sample obtained from an individual or from a tumor, is used as a template in an amplification reaction (e.g., Polymerase Chain Reaction (PCR)) using one or more oligonucleotides or primers, e.g., such as one or more oligonucleotides or primers provided herein. The presence of a biomarker nucleic acid molecule of the disclosure in the sample can be determined based on the presence or absence of an amplification product. Quantitative amplification methods are also known in the art and may be used according to the methods provided herein. Methods of measurement of DNA copy number at microsatellite loci using quantitative PCR analysis are known in the art. The known nucleotide sequence for genes is sufficient to enable one of skill in the art to routinely select primers to amplify any portion of the gene. Fluorogenic quantitative PCR can also be used. In fluorogenic quantitative PCR, quantitation is based on the amount of fluorescence signals, e.g., TaqMan and Sybr green.

Other amplification methods suitable for use according to the methods provided herein include, e.g., ligase chain reaction (LCR), transcription amplification, self-sustained sequence replication, dot PCR, and linker adapter PCR.

### Sequencing

In some embodiments, a biomarker nucleic acid molecule of the disclosure is detected in the RNA and/or DNA extracted from the de-embedded sample using a sequencing method. Any method of sequencing known in the art can be used to detect a biomarker nucleic acid molecule provided herein. Exemplary sequencing methods that may be used to detect a biomarker nucleic acid molecule provided herein include those based on techniques developed by Maxam and Gilbert or Sanger. Automated sequencing procedures may also be used, e.g., including sequencing by mass spectrometry.

In some embodiments, a biomarker nucleic acid molecule of the disclosure is detected in the RNA and/or DNA extracted from the de-embedded sample using hybrid capture-based sequencing (hybrid capture-based NGS), e.g., using adaptor ligation-based libraries. See, e.g., Frampton, G. M. et al. (2013) Nat. Biotech. 31:1023-1031. In some embodiments, a biomarker nucleic acid molecule of the disclosure is detected using next-generation sequencing (NGS). Next-generation sequencing includes any sequencing method that determines the nucleotide sequence of either individual nucleic acid molecules or clonally expanded proxies for individual nucleic acid molecules in a highly parallel fashion (e.g., greater than 10⁵ molecules may be sequenced simultaneously). Next generation sequencing methods suitable for use according to the methods provided herein are known in the art and include, without limitation, massively parallel short-read sequencing, template-based sequencing, pyrosequencing, real-time sequencing comprising imaging the continuous incorporation of dye-labeling nucleotides during DNA synthesis, nanopore sequencing, sequencing by hybridization, nano-transistor array based sequencing, polony sequencing, scanning tunneling microscopy (STM)-based sequencing, or nanowire-molecule sensor based sequencing. See, e.g., Metzker, M. (2010) Nature Biotechnology Reviews 11:31-46, which is hereby incorporated by reference. Exemplary NGS methods and platforms that may be used to detect a biomarker nucleic acid molecule provided herein include, without limitation, the HeliScope Gene Sequencing system from Helicos BioSciences (Cambridge, MA., USA), the PacBio RS system from Pacific Biosciences (Menlo Park, CA, USA), massively parallel short-read sequencing such as the Solexa sequencer and other methods and platforms from Illumina Inc. (San Diego, CA, USA), 454 sequencing from 454 LifeSciences (Branford, CT, USA), Ion Torrent sequencing from ThermoFisher (Waltham, MA, USA), or the SOLiD sequencer from Applied Biosystems (Foster City, CA, USA). Additional exemplary methods and platforms that may be used to detect a biomarker nucleic acid molecule provided herein include, without limitation, the Genome Sequencer (GS) FLX System from Roche (Basel, CHE), the G.007 polonator system, the Solexa Genome Analyzer, HiSeq 2500, HiSeq3000, HiSeq 4000, and NovaSeq 6000 platforms from Illumina Inc. (San Diego, CA, USA). In some embodiments, the one or more nucleic acids extracted from the RNA and/or DNA extracted from the de-embedded sample are analyzed by next-generation sequencing. In some embodiments, the method further comprises: e) optionally, ligating one or more adaptors onto the RNA and/or DNA extracted from the embedded sample, thereby generating ligated nucleic acids; d) optionally, amplifying nucleic acids from the ligated nucleic acids; f) optionally, capturing a plurality of nucleic acids corresponding to one or more genes of interest; g) sequencing, by a sequencer, the plurality of nucleic acids to obtain a plurality of sequence reads corresponding to the one or more genes of interest; h) analyzing the plurality of sequence reads; and i) based on the analysis, detecting one or more mutations in a gene of interest. In some embodiments, prior to step e) the one or more nucleic acids extracted from the sample are fragmented, optionally wherein the one or more nucleic acids extracted from the sample are fragmented by sonication. In some embodiments, the fragmented one or more nucleic acids extracted from the sample are end-repaired. In some embodiments, the end-repaired, fragmented one or more nucleic acids extracted from the sample are dA-tailed or dT-tailed. In some embodiments, the one or more nucleic acids extracted from the sample are prepared for sequencing according to the method described in Frampton, G. M. et al. (2013) Nat. Biotech. 31:1023-1031.

### Biomarker Detection

In some aspects, provided herein are reagents for detecting a biomarker nucleic acid molecule of the disclosure or a fragment thereof, e.g., in the nucleic acids (i.e., the RNA and/or DNA) extracted from the de-embedded samples, as described herein.

In general, RNA and/or DNA samples extracted from de-embedded samples by the methods described herein may be analyzed in order to determine the presence of a nucleic acid biomarker, such as a biomarker of cancer. Methods for determining the presence of particular nucleic acid biomarkers are described, for example in International Publication No. WO2021096888, U.S. Pat. Nos. 9,884,060, 9,297,011, 10,000,814, 8,673,972, 9,410,954, 9,907,798, U.S. Patent Publication No. 20160009785A1, U.S. Patent Publication No. 20200299775A1, U.S. Pat. Nos. 10,980,804, 9,861,633, U.S. Patent Publication No. 20180363066A1, U.S. Patent Publication No. 20170356053A1, U.S. Patent Publication No. 20190085403A1, U.S. Patent Publication No. 20190219586A1, International Publication No. WO2020243021, International Publication No. WO2021042066, International Application No. PCT/US2021/020752, International Application No. PCT/US2021/019982, International Publication No. WO2018009939, U.S. Provisional Patent Application No. 63/129,286, U.S. Provisional Patent Application No. 63/132,085, U.S. Provisional Patent Application No. 63/143,619, U.S. Provisional Patent Application No. 63/171,423, U.S. Provisional Patent Application No. 63/122,431, U.S. Provisional Patent Application No. 63/215,281, U.S. Provisional Patent Application No. 63/148,116, U.S. Provisional Patent Application No. 63/189,025, U.S. Provisional Patent Application No. 63/188,719, and U.S. Provisional Patent Application No. 63/215,356, each of which is hereby incorporated by reference in its entirety.

Further, exemplary nucleic acid biomarkers that may be detected in RNA and/or DNA samples extracted from de-embedded samples include loss-of heterozygosity (LOH), LOH of a human leukocyte antigen (HLA) gene (HLA LOH), loss-of-function of a phosphatase and tensin homolog (PTEN) gene (PTEN LOF), tumor mutational burden (TMB), and homozygous single exon loss, as described in detail below.

In some embodiments, a detection reagent provided herein comprises a nucleic acid molecule, e.g., a DNA, RNA, or mixed DNA/RNA molecule, comprising a nucleotide sequence that is complementary to a nucleotide sequence on a target nucleic acid, e.g., a nucleic acid that comprises a biomarker nucleic acid molecule described herein or a fragment or portion thereof. Provided herein are baits suitable for the detection of a biomarker nucleic acid molecule of the disclosure. In some embodiments, the bait comprises a capture nucleic acid molecule configured to hybridize to a target nucleic acid molecule comprising a biomarker nucleic acid molecule provided herein, or a fragment or portion thereof. In some embodiments, the capture nucleic acid molecule is configured to hybridize to the biomarker nucleic acid molecule of the target nucleic acid molecule. Also provided herein are probes, e.g., nucleic acid molecules, suitable for the detection of a biomarker nucleic acid molecule provided herein. In some embodiments, a probe provided herein comprises a nucleic acid sequence configured to hybridize to a target nucleic acid molecule comprising a biomarker nucleic acid molecule provided herein, or a fragment or portion thereof. In some embodiments, the probe comprises a nucleic acid sequence configured to hybridize to the biomarker nucleic acid molecule, or the fragment or portion thereof, of the target nucleic acid molecule. In some embodiments, the probe comprises a nucleic acid sequence configured to hybridize to a fragment or portion of the biomarker nucleic acid molecule of the target nucleic acid molecule. In some embodiments, the fragment or portion comprises between about 5 and about 25 nucleotides, between about 5 and about 300 nucleotides, between about 100 and about 300 nucleotides, between about 130 and about 230 nucleotides, or between about 150 and about 200 nucleotides.

### Loss-of-Heterozygosity (LOH) of One or More Genes of Interest, e.g., a Human Leukocyte Antigen (HLA) Gene

In some embodiments, provided herein are methods that comprise detecting loss-of-heterozygosity (LOH) of one or more genes of interest in RNA and/or DNA extracted from a de-embedded sample, as described herein. In some embodiments, provided herein are methods that comprise detecting LOH of a human leukocyte antigen (HLA) gene in RNA and/or DNA extracted from a de-embedded sample, as described herein. Exemplary methods of detecting LOH of a HLA gene are described in International Application No. PCT/US2021/019982, which is hereby incorporated by reference in its entirety.

In other embodiments, the gene of interest is ST7/RAY1, ARH1/NOEY2, TSLC1, RB, PTEN, SMAD2, SMAD4, DCC, TP53, ATM, miR-15a, miR-16-1, NAT2, BRCA1, BRCA2, hOGG1, CDH1, IGF2, CDKN1C/P57, MEN1, PRKAR1A, H19, KRAS, BAP1, PTCH1, SMO, SUFU, NOTCH1, PPP6C, LATS1, CASP8, PTPN14, ARID1A, FBXW7, M6P/IGF2R, IFN-alpha, an olfactory receptor gene, CBFA2T3, DUTT1, FHIT, APC, P16, FCMD, TSC2, miR-34, c-MPL, RUNX3, DIRAS3, NRAS, miR-9, FAM50B, PLAGL1, ER, FLT3, ZDBF2, GPR1, c-KIT, NAP1L5, GRB10, EGFR, PEG10, BRAF, MEST, JAK2, DAPK1, LIT1, WT1, NF-1, PR, c-CBL, DLK1, AKT1, SNURF, a cytochrome P450 gene (CYP), ZNF587, SOCS1, TIMP2, RUNX1, AR, CEBPA, C19MC, EMP3, ZNF331, CDKN2A, PEG3, NNAT, GNAS, or GATA5.

In some embodiments, any one of the methods described above further comprises detecting loss-of-heterozygosity (LOH) of a human leukocyte antigen (HLA) gene in RNA and/or DNA extracted from a de-embedded sample as described herein. In some embodiments according to any of the embodiments described herein, the HLA gene encodes a major histocompatibility (MHC) class I molecule. In some embodiments, the methods further comprise, after determining the adjusted allele frequency: determining that the gene has undergone loss-of-heterozygosity (LOH) based at least in part on the adjusted allele frequency.

In yet some other aspects, provided herein are methods for detecting loss-of-heterozygosity (LOH) of a human leukocyte antigen (HLA) gene in RNA and/or DNA extracted from a de-embedded sample as described herein. In some embodiments, the methods comprise: a) obtaining an observed allele frequency for an HLA allele, wherein observed allele frequency corresponds to frequency of nucleic acid(s) encoding at least a portion of the HLA allele as detected among a plurality of sequence reads corresponding to an HLA gene, wherein the plurality of sequence reads was obtained by sequencing nucleic acids encoding the gene or a portion thereof as captured by hybridization with a bait molecule; b) obtaining a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles; c) applying an objective function to measure a difference between the relative binding propensity and the observed allele frequency of the HLA allele; d) applying an optimization model to minimize the objective function; e) determining an adjusted allele frequency of the HLA allele based on the optimization model and the observed allele frequency; and f) determining that LOH has occurred when the adjusted allele frequency of the HLA allele is less than a predetermined threshold. In some embodiments, the HLA gene is a human HLA-A, HLA-B, or HLA-C gene. In some embodiments, the plurality of sequence reads was obtained by sequencing nucleic acids obtained from the RNA and/or DNA extracted from a de-embedded sample. In some embodiments, the methods are for detecting loss-of-heterozygosity (LOH) of a polymorphic gene of interest in the RNA and/or DNA extracted from a de-embedded sample. In some embodiments, the methods comprise: a) obtaining an observed allele frequency for an allele of a gene of interest, wherein observed allele frequency corresponds to frequency of nucleic acid(s) encoding at least a portion of the allele as detected among a plurality of sequence reads corresponding to the gene, wherein the plurality of sequence reads was obtained by sequencing nucleic acids encoding the gene or a portion thereof as captured by hybridization with a bait molecule; b) obtaining a relative binding propensity for the allele to the bait molecule, wherein the relative binding propensity of the allele corresponds to propensity of nucleic acid encoding at least a portion of the allele to bind the bait molecule in the presence of nucleic acids encoding portions of one or more other alleles; c) applying an objective function to measure a difference between the relative binding propensity and the observed allele frequency of the allele; d) applying an optimization model to minimize the objective function; e) determining an adjusted allele frequency of the allele based on the optimization model and the observed allele frequency; and f) determining that LOH has occurred when the adjusted allele frequency of the allele is less than a predetermined threshold. In some embodiments, the polymorphic gene is ST7/RAY1, ARH1/NOEY2, TSLC1, RB, PTEN, SMAD2, SMAD4, DCC, TP53, ATM, miR-15a, miR-16-1, NAT2, BRCA1, BRCA2, hOGG1, CDH1, IGF2, CDKN1C/P57, MEN1, PRKAR1A, H19, KRAS, BAP1, PTCH1, SMO, SUFU, NOTCH1, PPP6C, LATS1, CASP8, PTPN14, ARID1A, FBXW7, M6P/IGF2R, IFN-alpha, an olfactory receptor gene, CBFA2T3, DUTT1, FHIT, APC, P16, FCMD, TSC2, miR-34, c-MPL, RUNX3, DIRAS3, NRAS, miR-9, FAM50B, PLAGL1, ER, FLT3, ZDBF2, GPR1, c-KIT, NAP1L5, GRB10, EGFR, PEG10, BRAF, MEST, JAK2, DAPK1, LIT1, WT1, NF-1, PR, c-CBL, DLK1, AKT1, SNURF, a cytochrome P450 gene (CYP), ZNF587, SOCS1, TIMP2, RUNX1, AR, CEBPA, C19MC, EMP3, ZNF331, CDKN2A, PEG3, NNAT, GNAS, or GATA5.

In yet some other aspects, any of the methods of the present disclosure further comprise measuring TMB, e.g., in RNA and/or DNA extracted from a de-embedded sample as described herein. In some embodiments, the methods comprise determining LOH and assessing TMB, e.g., in RNA and/or DNA extracted from a de-embedded sample. As demonstrated herein, HLA LOH and high TMB (and optionally intact HLA gene(s)) may be predictive of increased overall survival, increased probability of greater survival, and/or increased likelihood of response to ICI therapy, e.g., as compared to HLA LOH without high TMB. In some embodiments, high TMB refers to a TMB of greater than or equal to 10 mutations/Mb or greater than or equal to 13 mutations/Mb. In some embodiments, TMB is obtained from a plurality of sequence reads, e.g., a plurality of sequence reads obtained by sequencing nucleic acids at least a portion of a genome (such as from an enriched or unenriched sample). In some embodiments, TMB is determined based on a number of non-driver somatic coding mutations per megabase of genome sequenced.

In some embodiments, any of the methods of the present disclosure comprise acquiring knowledge of LOH of the HLA gene (e.g., in RNA and/or DNA extracted from a de-embedded sample) and acquiring knowledge of TMB (e.g., in RNA and/or DNA extracted from a de-embedded sample). In some embodiments, any of the methods of the present disclosure comprise detecting LOH of the HLA gene (e.g., in RNA and/or DNA extracted from a de-embedded sample) and acquiring knowledge of TMB (e.g., in RNA and/or DNA extracted from a de-embedded sample). In some embodiments, any of the methods of the present disclosure comprise acquiring knowledge of LOH of the HLA gene (e.g., in RNA and/or DNA extracted from a de-embedded sample) and detecting or determining TMB (e.g., in RNA and/or DNA extracted from a de-embedded sample). In some embodiments, any of the methods of the present disclosure comprise detecting LOH of the HLA gene (e.g., in RNA and/or DNA extracted from a de-embedded sample) and detecting or determining TMB (e.g., in RNA and/or DNA extracted from a de-embedded sample). In some embodiments, the samples used to detect/determine LOH and TMB are the same. In some embodiments, the samples used to detect/determine LOH and TMB are different.

### Phosphatase and Tensin Homolog (PTEN)

Phosphatase and tensin homolog (PTEN) deleted on chromosome 10 is one of the most frequently disrupted tumor suppressors in cancer. The lipid phosphatase activity of PTEN antagonizes the phosphatidylinositol 3-kinase (PI3K)/AKT/mTOR pathway to repress tumor cell growth and survival. Accordingly, a loss-of-function mutation in a PTEN gene can serve as a biomarker for cancer.

In some embodiments, provided herein are methods that comprise detecting a loss-of-function mutation in a phosphatase and tensin homolog (PTEN) gene in RNA and/or DNA extracted from a de-embedded sample as described herein. In some embodiments, the loss-of-function mutation in the PTEN gene comprises one or more of an insertion, deletion or substitution of one or more nucleotides, a genomic rearrangement, an alteration in a promoter, a gene fusion, or a copy number alteration.

### Tumor Mutational Burden (TMB)

In some embodiments, provided herein are methods that comprise measuring the level of tumor mutational burden (TMB) in RNA and/or DNA extracted from a de-embedded sample as described herein. In some embodiments, the methods provided herein comprise acquiring knowledge that a RNA and/or DNA extracted from a de-embedded sample has a tumor mutational burden of at least about 10 mut/Mb or at least about 20 mut/Mb. In some embodiments, acquiring knowledge that the RNA and/or DNA extracted from a de-embedded sample has a tumor mutational burden of at least about 10 mut/Mb or at least about 20 mut/Mb comprises measuring the level of tumor mutational burden in RNA and/or DNA extracted from a de-embedded sample, e.g., in a RNA and/or DNA extracted from a de-embedded sample, wherein the embedded sample was obtained from an individual. In some embodiments, the methods provided herein comprise detecting a tumor mutational burden of at least about 10 mut/Mb or at least about 20 mut/Mb in a RNA and/or DNA extracted from a de-embedded sample. In some embodiments, the methods comprise administering an effective amount of an immunotherapy responsive to knowledge that the RNA and/or DNA extracted from a de-embedded sample has a tumor mutational burden of at least about 10 mut/Mb or at least about 20 mut/Mb. In some embodiments, the methods comprise providing a report to a party.

In some embodiments, tumor mutational burden is assessed in RNA and/or DNA extracted from a de-embedded sample derived from an individual. In some embodiments, the embedded sample from the individual comprises a tumor biopsy. In some embodiments, the embedded sample from the individual comprises nucleic acids.

In some embodiments, tumor mutational burden is measured using any suitable method known in the art. For example, tumor mutational burden may be measured using whole-exome sequencing (WES), next-generation sequencing, whole genome sequencing, gene-targeted sequencing, or sequencing of a panel of genes, e.g., panels including cancer-related genes. See, e.g., Melendez et al., Transl Lung Cancer Res (2018) 7(6):661-667. In some embodiments, tumor mutational burden is measured using gene-targeted sequencing, e.g., using a nucleic acid hybridization-capture method, e.g., coupled with sequencing. See, e.g., Fancello et al., J Immunother Cancer (2019) 7:183.

In some embodiments, tumor mutational burden is measured according to the methods provided in WO2017151524A1, which is hereby incorporated by reference in its entirety.

In some embodiments, tumor mutational burden is measured in the RNA and/or DNA extracted from a de-embedded sample by whole exome sequencing. In some embodiments, tumor mutational burden is measured in the RNA and/or DNA extracted from a de-embedded sample using next-generation sequencing. In some embodiments, tumor mutational burden is measured in the RNA and/or DNA extracted from a de-embedded sample using whole genome sequencing. In some embodiments, tumor mutational burden is measured in the RNA and/or DNA extracted from a de-embedded sample by gene-targeted sequencing. In some embodiments, tumor mutational burden is measured on between about 0.8 Mb and about 1.1 Mb of sequenced DNA. In some embodiments, tumor mutational burden is measured on any of about 0.8 Mb, about 0.81 Mb, about 0.82 Mb, about 0.83 Mb, about 0.84 Mb, about 0.85 Mb, about 0.86 Mb, about 0.87 Mb, about 0.88 Mb, about 0.89 Mb, about 0.9 Mb, about 0.91 Mb, about 0.92 Mb, about 0.93 Mb, about 0.94 Mb, about 0.95 Mb, about 0.96 Mb, about 0.97 Mb, about 0.98 Mb, about 0.99 Mb, about 1 Mb, about 1.01 Mb, about 1.02 Mb, about 1.03 Mb, about 1.04 Mb, about 1.05 Mb, about 1.06 Mb, about 1.07 Mb, about 1.08 Mb, about 1.09 Mb, or about 1.1 Mb of sequenced DNA. In some embodiments, tumor mutational burden is measured on about 0.8 Mb of sequenced DNA.

In some embodiments, the RNA and/or DNA extracted from a de-embedded sample has a high tumor mutational burden, e.g., of at least about 10 mut/Mb. In some embodiments, the RNA and/or DNA extracted from a de-embedded sample has a tumor mutational burden of at least about 10 mut/Mb. In some embodiments, the RNA and/or DNA extracted from a de-embedded sample has a tumor mutational burden of at least about 20 mut/Mb. In some embodiments, the RNA and/or DNA extracted from a de-embedded sample has a tumor mutational burden of any of between about 10 mut/Mb and about 15 mut/Mb, between about 15 mut/Mb and about 20 mut/Mb, between about 20 mut/Mb and about 25 mut/Mb, between about 25 mut/Mb and about 30 mut/Mb, between about 30 mut/Mb and about 35 mut/Mb, between about 35 mut/Mb and about 40 mut/Mb, between about 40 mut/Mb and about 45 mut/Mb, between about 45 mut/Mb and about 50 mut/Mb, between about 50 mut/Mb and about 55 mut/Mb, between about 55 mut/Mb and about 60 mut/Mb, between about 60 mut/Mb and about 65 mut/Mb, between about 65 mut/Mb and about 70 mut/Mb, between about 70 mut/Mb and about 75 mut/Mb, between about 75 mut/Mb and about 80 mut/Mb, between about 80 mut/Mb and about 85 mut/Mb, between about 85 mut/Mb and about 90 mut/Mb, between about 90 mut/Mb and about 95 mut/Mb, or between about 95 mut/Mb and about 100 mut/Mb. In some embodiments, the RNA and/or DNA extracted from a de-embedded sample has a tumor mutational burden of any of between about 100 mut/Mb and about 110 mut/Mb, between about 110 mut/Mb and about 120 mut/Mb, between about 120 mut/Mb and about 130 mut/Mb, between about 130 mut/Mb and about 140 mut/Mb, between about 140 mut/Mb and about 150 mut/Mb, between about 150 mut/Mb and about 160 mut/Mb, between about 160 mut/Mb and about 170 mut/Mb, between about 170 mut/Mb and about 180 mut/Mb, between about 180 mut/Mb and about 190 mut/Mb, between about 190 mut/Mb and about 200 mut/Mb, between about 210 mut/Mb and about 220 mut/Mb, between about 220 mut/Mb and about 230 mut/Mb, between about 230 mut/Mb and about 240 mut/Mb, between about 240 mut/Mb and about 250 mut/Mb, between about 250 mut/Mb and about 260 mut/Mb, between about 260 mut/Mb and about 270 mut/Mb, between about 270 mut/Mb and about 280 mut/Mb, between about 280 mut/Mb and about 290 mut/Mb, between about 290 mut/Mb and about 300 mut/Mb, between about 300 mut/Mb and about 310 mut/Mb, between about 310 mut/Mb and about 320 mut/Mb, between about 320 mut/Mb and about 330 mut/Mb, between about 330 mut/Mb and about 340 mut/Mb, between about 340 mut/Mb and about 350 mut/Mb, between about 350 mut/Mb and about 360 mut/Mb, between about 360 mut/Mb and about 370 mut/Mb, between about 370 mut/Mb and about 380 mut/Mb, between about 380 mut/Mb and about 390 mut/Mb, between about 390 mut/Mb and about 400 mut/Mb, or more than 400 mut/Mb.

In some embodiments, measuring tumor mutational burden comprises assessing mutations in RNA and/or DNA extracted from a de-embedded sample derived from a cancer in an individual. In some embodiments, measuring tumor mutational burden comprises assessing mutations in RNA and/or DNA extracted from a de-embedded sample derived from a cancer in an individual, and in a matched normal sample, e.g., RNA and/or DNA extracted from a de-embedded sample from the individual derived from a tissue or other source that is free of the cancer.

### Homozygous Single Exon Loss

In general, homozygous single exon loss refers to the deletion of both copies of a given exon. In some embodiments, provided herein are methods that comprise detecting homozygous single exon loss in RNA and/or DNA extracted from a de-embedded sample. In some embodiments, the homozygous single exon loss is detected in RNA and/or DNA extracted from a de-embedded sample by whole exome sequencing, whole genome sequencing, or gene-targeted sequencing

### Systems, Software, and Devices

In some other aspects, provided herein are non-transitory computer-readable storage media. In some embodiments, the non-transitory computer-readable storage media comprise one or more programs for execution by one or more processors of a device, the one or more programs including instructions which, when executed by the one or more processors, cause the device to perform the method according to any of the embodiments described herein.

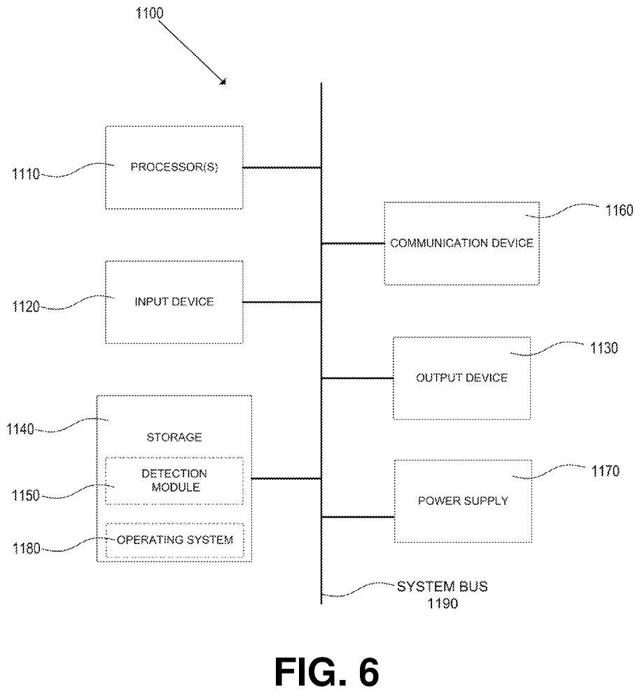

FIG. 6 illustrates an example of a computing device in accordance with one embodiment. Device 1100 can be a host computer connected to a network. Device 1100 can be a client computer or a server. As shown in FIG. 6, device 1100 can be any suitable type of microprocessor-based device, such as a personal computer, workstation, server or handheld computing device (portable electronic device) such as a phone or tablet. The device can include, for example, one or more of processor(s) 1110, input device 1120, output device 1130, storage 1140, communication device 1160, power supply 1170, operating system 1180, and system bus 1190. Input device 1120 and output device 1130 can generally correspond to those described herein, and can either be connectable or integrated with the computer.

Input device 1120 can be any suitable device that provides input, such as a touch screen, keyboard or keypad, mouse, or voice-recognition device. Output device 1130 can be any suitable device that provides output, such as a touch screen, haptics device, or speaker.

Storage 1140 can be any suitable device that provides storage (e.g., an electrical, magnetic or optical memory including a RAM (volatile and non-volatile), cache, hard drive, or removable storage disk). Communication device 1160 can include any suitable device capable of transmitting and receiving signals over a network, such as a network interface chip or device. The components of the computer can be connected in any suitable manner, such as via a wired media (e.g., a physical bus, ethernet, or any other wire transfer technology) or wirelessly (e.g., Bluetooth®, Wi-Fi®, or any other wireless technology). For example, in FIG. 6, the components are connected by System Bus 1190.

Detection module 1150, which can be stored as executable instructions in storage 1140 and executed by processor(s) 1110, can include, for example, the processes that embody the functionality of the present disclosure (e.g., as embodied in the devices as described herein).

Detection module 1150 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 1140, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Detection module 1150 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

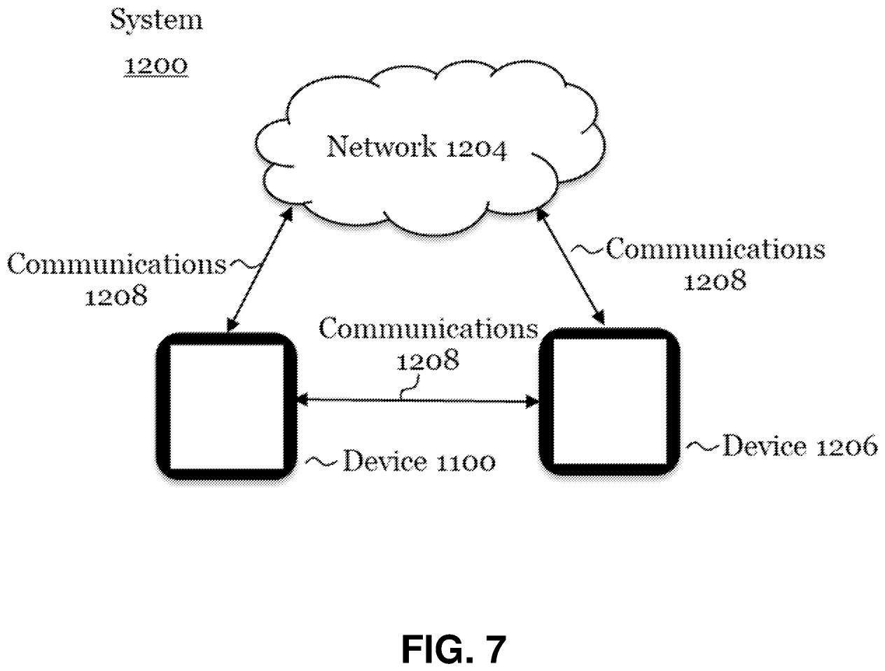

Device 1100 may be connected to a network (e.g., Network 1204, as shown in FIG. 7 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 1100 can implement any operating system (e.g., Operating System 1180) suitable for operating on the network. Detection module 1150 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, Operating System 1180 is executed by one or more processors, e.g., Processor(s) 1110.

Device 1100 can further include Power Supply 1170, which can be any suitable power supply.

FIG. 7 illustrates an example of a computing system in accordance with one embodiment. In System 1200, Device 1100 (e.g., as described above and illustrated in FIG. 6) is connected to Network 1204, which is also connected to Device 1206. In some embodiments, Device 1206 is a sequencer. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system.

Devices 1100 and 1206 may communicate, e.g., using suitable communication interfaces via Network 1204, such as a Local Area Network (LAN), Virtual Private Network (VPN), or the Internet. In some embodiments, Network 1204 can be, for example, the Internet, an intranet, a virtual private network, a cloud network, a wired network, or a wireless network. Devices 1100 and 1206 may communicate, in part or in whole, via wireless or hardwired communications, such as Ethernet, IEEE 802.11b wireless, or the like. Additionally, Devices 1100 and 1206 may communicate, e.g., using suitable communication interfaces, via a second network, such as a mobile/cellular network. Communication between Devices 1100 and 1206 may further include or communicate with various servers such as a mail server, mobile server, media server, telephone server, and the like. In some embodiments, Devices 1100 and 1206 can communicate directly (instead of, or in addition to, communicating via Network 1204), e.g., via wireless or hardwired communications, such as Ethernet, IEEE 802.11b wireless, or the like. In some embodiments, Devices 1100 and 1206 communicate via Communications 1208, which can be a direct connection or can occur via a network (e.g., Network 1204).

One or all of Devices 1100 and 1206 generally include logic (e.g., http web server logic) or is programmed to format data, accessed from local or remote databases or other sources of data and content, for providing and/or receiving information via Network 1204 according to various examples described herein.

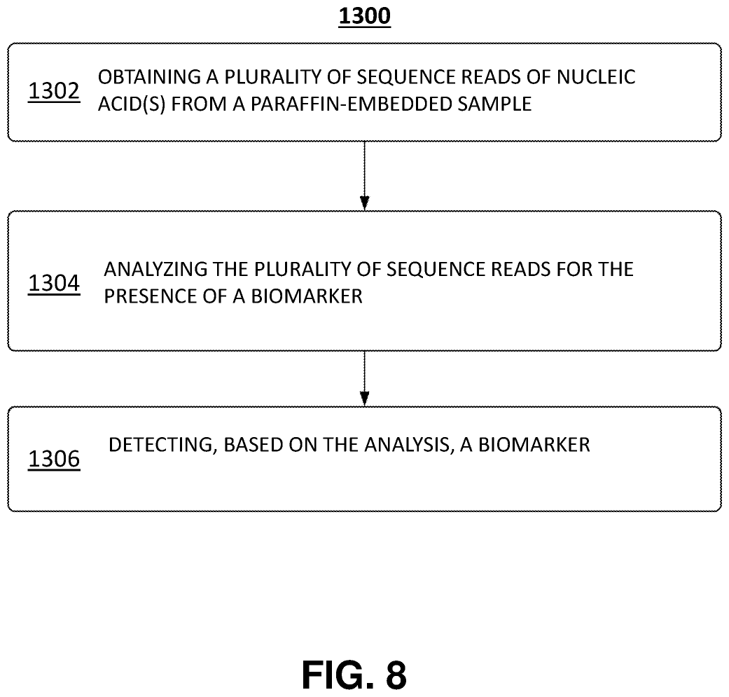

FIG. 8 illustrates an exemplary process 1300 for detecting a biomarker in an analyte sample extracted from an embedded sample, in accordance with some embodiments. Process 1300 is performed, for example, using one or more electronic devices implementing a software program. In some examples, process 1300 is performed using a client-server system, and the blocks of process 1300 are divided up in any manner between the server and a client device. In other examples, the blocks of process 1300 are divided up between the server and multiple client devices. Thus, while portions of process 1300 are described herein as being performed by particular devices of a client-server system, it will be appreciated that process 1300 is not so limited. In some embodiments, the executed steps can be executed across many systems, e.g., in a cloud environment. In other examples, process 1300 is performed using only a client device or only multiple client devices. In process 1300, some blocks are, optionally, combined, the order of some blocks is, optionally, changed, and some blocks are, optionally, omitted. In some examples, additional steps may be performed in combination with the process 1300. Accordingly, the operations as illustrated (and described in greater detail below) are exemplary by nature and, as such, should not be viewed as limiting.

At block 1302, a plurality of sequence reads of one or more nucleic acids is obtained, wherein the one or more nucleic acids are derived from a sample extracted from a de-embedded obtained from an individual. In some embodiments, the sample is obtained from an individual having a cancer, such as a cancer described herein. In some embodiments, the sequence reads are obtained using a sequencer, e.g., as described herein or otherwise known in the art. In some embodiments, the nucleic acid(s) comprise one or more nucleic acids corresponding to a biomarker of the present disclosure, or portion thereof. Optionally, prior to obtaining the sequence reads, the sample is purified, enriched (e.g., for nucleic acid(s) corresponding to a biomarker gene of the present disclosure, or portion thereof), and/or subjected to PCR amplification. At block 1304, an exemplary system (e.g., one or more electronic devices) analyzes the plurality of sequence reads for the presence of one or more mutations in a biomarker, or a portion thereof. At block 1306, the system detects (e.g., based on the analysis) one or more mutations in a biomarker, or a portion thereof, in the sample.

### V. Diagnostic and Therapeutic Methods and Kits Methods of Diagnosing, Assessing, Screening, Monitoring or Predicting

In some aspects, provided herein are methods of diagnosing or assessing a biomarker in a cancer, such as a cancer provided herein, in an individual. In some embodiments, the methods comprise acquiring knowledge of the presence of a nucleic acid molecule provided herein in an RNA and/or DNA sample extracted from an embedded sample obtained from the individual. In some embodiments, the methods comprise detecting a biomarker nucleic acid molecule provided herein in in an RNA and/or DNA sample extracted from an embedded sample obtained from the individual. In some embodiments, the biomarker nucleic acid molecule is detected in an RNA and/or DNA sample extracted from an embedded sample obtained from the individual using any method known in the art, such as one or more of the methods of detection of biomarker nucleic acid molecules described herein. In some embodiments, the methods further comprise providing a diagnosis or an assessment of the biomarker nucleic acid molecule. In some embodiments, the diagnosis or assessment identifies the presence or absence of the biomarker nucleic acid molecule in the RNA and/or DNA sample extracted from an embedded sample. In some embodiments, the diagnosis or assessment identifies the cancer, such as a cancer provided herein, as likely to respond to an anti-cancer therapy, e.g., an anti-cancer therapy provided herein. In some embodiments, the presence of the biomarker nucleic acid molecule in the RNA and/or DNA sample extracted from an embedded sample identifies the cancer as likely to respond to an anti-cancer therapy, e.g., an anti-cancer therapy provided herein. In some embodiments, the RNA and/or DNA sample extracted from an embedded sample is a sample described herein. In some embodiments, the embedded sample comprises cells from the cancer or is obtained from cells from the cancer. In some embodiments, the individual has a cancer, is suspected of having a cancer, is being tested for a cancer, is being treated for a cancer, or is being tested for a susceptibility to a cancer, e.g., a cancer described herein.

In some aspects, provided herein are methods of diagnosing or assessing a cancer in an individual, e.g., a cancer provided herein. In some embodiments, the methods of diagnosing or assessing cancer comprise detecting a biomarker nucleic acid molecule provided herein in a RNA and/or DNA sample extracted from an embedded sample obtained from the individual, e.g., an embedded sample comprising cells from the cancer. In some embodiments, the methods comprise detecting a biomarker nucleic acid molecule described herein in an RNA and/or DNA sample extracted from an embedded sample obtained from the individual using any method known in the art, such as one or more of the methods of detection of biomarker nucleic acid molecules described herein. In some embodiments, detection of a biomarker nucleic acid molecule described herein, or a fragment thereof, in an RNA and/or DNA sample extracted from an embedded sample obtained from the individual identifies the cancer as likely to respond to an anti-cancer therapy, e.g., an anti-cancer therapy provided herein. In some embodiments, the presence of a biomarker nucleic acid molecule described herein, or a fragment thereof, in an RNA and/or DNA sample extracted from an embedded sample obtained from the individual identifies the cancer as likely to respond to an anti-cancer therapy, e.g., an anti-cancer therapy provided herein. In some embodiments, the methods further comprise providing a diagnosis or an assessment of the cancer or of the fusion nucleic acid molecule. In some embodiments, the diagnosis or assessment identifies the cancer as likely to respond to an anti-cancer therapy, e.g., an anti-cancer therapy provided herein. In some embodiments, the diagnosis or assessment identifies the presence or absence of the biomarker nucleic acid molecule in the RNA and/or DNA sample extracted from an embedded sample.

In some aspects, provided herein are methods of predicting survival of an individual having a cancer, e.g., a cancer provided herein. In some embodiments, the individual is being treated with an anti-cancer therapy, such as an anti-cancer therapy described herein. In some embodiments, the methods comprise acquiring knowledge of a biomarker nucleic acid molecule provided herein in RNA and/or DNA sample extracted from an embedded sample from the individual. In some embodiments, the methods comprise detecting a biomarker nucleic acid molecule provided herein in RNA and/or DNA sample extracted from an embedded sample from the individual. In some embodiments, responsive to acquiring knowledge of a biomarker nucleic acid molecule provided herein in the RNA and/or DNA sample extracted from an embedded sample, the individual is predicted to have longer survival after treatment with an anti-cancer therapy, e.g., an anti-cancer therapy provided herein, for example, as compared to an individual whose cancer does not exhibit the biomarker nucleic acid molecule. In some embodiments, responsive to detecting a biomarker nucleic acid molecule provided herein in the RNA and/or DNA sample extracted from an embedded sample, the individual is predicted to have longer survival after treatment with an anti-cancer therapy, e.g., an anti-cancer therapy provided herein, for example, as compared to an individual whose cancer does not exhibit the biomarker nucleic acid molecule. In some embodiments, the methods further comprise providing a diagnosis or an assessment. In some embodiments, the diagnosis or assessment identifies the presence or absence of the biomarker nucleic acid molecule in the RNA and/or DNA sample extracted from an embedded sample. In some embodiments, the diagnosis or assessment identifies the individual as being predicted to have longer survival after treatment with an anti-cancer therapy, e.g., an anti-cancer therapy provided herein, for example, as compared to an individual whose cancer does not exhibit the biomarker nucleic acid molecule. In some embodiments, the RNA and/or DNA sample extracted from an embedded sample is a sample as described herein. In some embodiments, the embedded sample comprises cells from the cancer.

In some aspects, provided herein are methods of screening an individual having cancer, suspected of having cancer, being tested for cancer, being treated for cancer, or being tested for a susceptibility to cancer, e.g., a cancer provided herein. In some embodiments, the individual is being treated with an anti-cancer therapy, such as an anti-cancer therapy described herein. In some embodiments, the methods comprise acquiring knowledge of a biomarker nucleic acid molecule provided herein in a RNA and/or DNA sample extracted from an embedded sample from the individual. In some embodiments, the methods comprise detecting a biomarker nucleic acid molecule provided herein in a RNA and/or DNA sample extracted from an embedded sample from the individual. In some embodiments, responsive to acquiring knowledge of a biomarker nucleic acid molecule provided herein in the RNA and/or DNA sample extracted from an embedded sample, the individual is predicted to have increased risk of cancer recurrence, aggressive cancer, anti-cancer therapy resistance, or poor prognosis, for example, as compared to an individual whose cancer does not exhibit the biomarker nucleic acid molecule. In some embodiments, responsive to detecting a biomarker nucleic acid molecule provided herein in the RNA and/or DNA sample extracted from an embedded sample extracted from a tissue, the individual is predicted to have increased risk of cancer recurrence, aggressive cancer, anti-cancer therapy resistance, or poor prognosis, for example, as compared to an individual whose cancer does not exhibit the biomarker nucleic acid molecule. In some embodiments, the methods further comprise providing a diagnosis or an assessment. In some embodiments, the diagnosis or assessment identifies the presence or absence of the biomarker nucleic acid molecule in the RNA and/or DNA sample extracted from an embedded sample extracted from a tissue. In some embodiments, the diagnosis or assessment identifies the individual as being predicted to have increased risk of cancer recurrence, aggressive cancer, anti-cancer therapy resistance, or poor prognosis, for example, as compared to an individual whose cancer does not exhibit the biomarker nucleic acid molecule. In some embodiments, the RNA and/or DNA sample extracted from an embedded sample is a as described herein. In some embodiments, the embedded sample comprises cells from the cancer.

In some embodiments, the methods further comprise selectively enriching for one or more nucleic acids comprising biomarker nucleotide sequences to produce an enriched sample, e.g., using a reagent known in the art or provided herein, such as a bait, probe, or oligonucleotide described herein.

### Anti-Cancer Therapies Cancers

Certain aspects of the present disclosure relate to anti-cancer therapies, as well as methods for identifying an individual who may benefit from treatment with an anti-cancer therapy, methods for selecting an anti-cancer therapy for treating an individual, methods for identifying an anti-cancer therapy as a treatment option, methods for treating or delaying progression of cancer comprising administration of an anti-cancer therapy, uses for anti-cancer therapies (e.g., in methods of treating or delaying progression of cancer in an individual, or in methods for manufacturing a medicament for treating or delaying progression of cancer), and the like. These methods and uses are based, at least in part, on the detection of biomarkers from tumor cells of interest as in embedded samples, as described above. Without wishing to be bound to theory, it is thought that these biomarkers can identify patients that would benefit from appropriate anti-cancer therapies such as one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

In some embodiments, the anti-cancer therapy comprises a cyclin-dependent kinase (CDK) inhibitor. In some embodiments, the CDK inhibitor inhibits CDK4. In some embodiments, the CDK inhibitor inhibits Cyclin D/CDK4. In some embodiments, the anti-cancer therapy/CDK inhibitor is (a) a small molecule that inhibits one or more enzymatic activities of CDK4, (b) an antibody that inhibits one or more activities of CDK4 (e.g., by binding to and inhibiting one or more activities of CDK4, binding to and inhibiting expression of CDK4, and/or binding to and inhibiting one or more activities of a cell expressing CDK4, such as by inducing antibody-dependent cellular cytotoxicity, ADCC, or phagocytosis, ADCP), or (c) a nucleic acid that inhibits expression of CDK4 (e.g., an antisense oligonucleotide, miRNA, siRNA, morpholino, CRISPR-based therapeutic, and the like). In some embodiments, the CDK inhibitor inhibits CDK4 and CDK6. In some embodiments, the CDK inhibitor is a small molecule inhibitor of CDK4 (e.g., a competitive or non-competitive inhibitor). Non-limiting examples of CDK inhibitors include palbociclib, ribociclib, and abemaciclib, as well as pharmaceutically acceptable salts thereof.

In some embodiments, the anti-cancer therapy comprises a murine double minute 2 homolog (MDM2) inhibitor. In some embodiments, the anti-cancer therapy/MDM2 inhibitor is (a) a small molecule that inhibits one or more activities of MDM2 (e.g., binding to p53), (b) an antibody that inhibits one or more activities of MDM2 (e.g., by binding to and inhibiting one or more activities of MDM2, binding to and inhibiting expression of MDM2, and/or binding to and inhibiting one or more activities of a cell expressing MDM2, such as by inducing antibody-dependent cellular cytotoxicity, ADCC, or phagocytosis, ADCP), or (c) a nucleic acid that inhibits expression of MDM2 (e.g., an antisense oligonucleotide, miRNA, siRNA, morpholino, CRISPR-based therapeutic, and the like). In some embodiments, the MDM2 inhibitor is a small molecule inhibitor of MDM2 (e.g., a competitive or non-competitive inhibitor). Non-limiting examples of MDM2 inhibitors include nutlin-3a, RG7112, idasanutlin (RG7388), AMG-232, MI-63, MI-291, MI-391, MI-77301 (SAR405838), APG-115, DS-3032b, NVP-CGM097, and HDM-201 (siremadlin), as well as pharmaceutically acceptable salts thereof. In some embodiments, the MDM2 inhibitor inhibits or disrupts interaction between MDM2 and p53.

In some embodiments, the anti-cancer therapy comprises one or more of an antimetabolite, DNA-damaging agent, or platinum-containing therapeutic (e.g., 5-azacitadine, 5-fluorouracil, acadesine, busulfan, carboplatin, cisplatin, chlorambucil, CPT-11, cytarabine, daunorubicin, decitabine, doxorubicin, etoposide, fludarabine, gemcitabine, idarubicin, radiation, oxaliplatin, temozolomide, topotecan, trabectedin, GSK2830371, or rucaparib); a pro-apoptotic agent (e.g., a BCL2 inhibitor or downregulator, SMAC mimetic, or TRAIL agonist such as ABT-263, ABT-737, oridonin, venetoclax, combination of venetoclax and an anti-CD20 antibody such as obinutuzumab or rituximab, 1396-11, ABT-10, SM-164, D269H/E195R, or rhTRAIL); a tyrosine kinase inhibitor (e.g., as described herein); an inhibitor of RAS, RAF, MEK, or the MAPK pathway (e.g., AZD6244, dabrafenib, LGX818, PD0325901, pimasertib, trametinib, or vemurafenib); an inhibitor of PI3K, mTOR, or Akt (e.g., as described herein); a CDK inhibitor (e.g., as described herein); a PKC inhibitor (e.g., LXS196 or sotrastaurin); an antibody-based therapeutic (e.g., an anti-PD-1 or anti-PDL1 antibody such as atezolizumab, pembrolizumab, nivolumab, or spartalizumab; an anti-CD20 antibody such as obinutuzumab or rituximab; or an anti-DR5 antibody such as drozitumab); a proteasome inhibitor (e.g., bortezomib, carfilzomib, ixazomib, or MG-132); an HDAC inhibitor (e.g., SAHA or VPA); an antibiotic (e.g., actinomycin D); a zinc-containing therapeutic (e.g., zinc or ZMC1); an HSP inhibitor (e.g., geldanamycin); an ATPase inhibitor (e.g., archazolid); a mitotic inhibitor (e.g., paclitaxel or vincristine); metformin; methotrexate; tanshinone IIA; and/or P5091.

In some embodiments, the anti-cancer therapy comprises a tyrosine kinase inhibitor. In some embodiments, the anti-cancer therapy/tyrosine kinase inhibitor is (a) a small molecule that inhibits one or more enzymatic activities of a tyrosine kinase, (b) an antibody that inhibits one or more activities of a tyrosine kinase (e.g., by binding to and inhibiting one or more activities of the tyrosine kinase, binding to and inhibiting expression, such as cell surface expression, of the tyrosine kinase, and/or binding to and inhibiting one or more activities of a cell expressing the tyrosine kinase, such as by inducing antibody-dependent cellular cytotoxicity, ADCC, or phagocytosis, ADCP), or (c) a nucleic acid that inhibits expression of a tyrosine kinase (e.g., an antisense oligonucleotide, miRNA, siRNA, morpholino, CRISPR-based therapeutic, and the like). In some embodiments, the tyrosine kinase inhibitor is a small molecule inhibitor of a tyrosine kinase (e.g., a competitive or non-competitive inhibitor). Non-limiting examples of tyrosine kinase inhibitors include imatinib, crenolanib, linifanib, ninetedanib, axitinib, dasatinib, imetelstat, midostaurin, pazopanib, sorafenib, sunitinb, motesanib, masitinib, vatalanib, cabozanitinib, tivozanib, OSI-930, Ki8751, telatinib, dovitinib, tyrphostin AG 1296, and amuvatinib, as well as pharmaceutically acceptable salts thereof.

In some embodiments, the anti-cancer therapy comprises a mitogen-activated protein kinase (MEK) inhibitor. In some embodiments, the MEK inhibitor inhibits one or more activities of MEK1 and/or MEK2. In some embodiments, the anti-cancer therapy/MEK inhibitor is (a) a small molecule that inhibits one or more enzymatic activities of MEK, (b) an antibody that inhibits one or more activities of MEK (e.g., by binding to and inhibiting one or more activities of MEK, binding to and inhibiting expression of MEK, and/or binding to and inhibiting one or more activities of a cell expressing MEK, such as by inducing antibody-dependent cellular cytotoxicity, ADCC, or phagocytosis, ADCP), or (c) a nucleic acid that inhibits expression of MEK (e.g., an antisense oligonucleotide, miRNA, siRNA, morpholino, CRISPR-based therapeutic, and the like). In some embodiments, the MEK inhibitor is a small molecule inhibitor of MEK (e.g., a competitive or non-competitive inhibitor). Non-limiting examples of MEK inhibitors include trametinib, cobimetinib, binimetinib, CI-1040, PD0325901, selumetinib, AZD8330, TAK-733, GDC-0623, refametinib, pimasertib, R04987655, RO5126766, WX-544, and HL-085, as well as pharmaceutically acceptable salts thereof. In some embodiments, the anti-cancer therapy inhibits one or more activities of the Raf/MEK/ERK pathway, including inhibitors of Raf, MEK, and/or ERK.

In some embodiments, the anti-cancer therapy comprises a mammalian target of rapamycin (mTOR) inhibitor. In some embodiments, the anti-cancer therapy/mTOR inhibitor is (a) a small molecule that inhibits one or more enzymatic activities of mTOR, (b) an antibody that inhibits one or more activities of mTOR (e.g., by binding to and inhibiting one or more activities of mTOR, binding to and inhibiting expression of mTOR, and/or binding to and inhibiting one or more activities of a cell expressing mTOR, such as by inducing antibody-dependent cellular cytotoxicity, ADCC, or phagocytosis, ADCP), or (c) a nucleic acid that inhibits expression of mTOR (e.g., an antisense oligonucleotide, miRNA, siRNA, morpholino, CRISPR-based therapeutic, and the like). In some embodiments, the mTOR inhibitor is a small molecule inhibitor of mTOR (e.g., a competitive inhibitor, such as an ATP-competitive inhibitor, or a non-competitive inhibitor, such as a rapamycin analog). Non-limiting examples of mTOR inhibitors include temsirolimus, everolimus, ridaforolimus, dactolisib, GSK2126458, XL765, AZD8055, AZD2014, MLN128, PP242, NVP-BEZ235, LY3023414, PQR309, PKI587, and OSI027, as well as pharmaceutically acceptable salts thereof. In some embodiments, the anti-cancer therapy inhibits one or more activities of the Akt/mTOR pathway, including inhibitors of Akt and/or mTOR.

In some embodiments, the anti-cancer therapy comprises a PI3K inhibitor or Akt inhibitor. In some embodiments, the PI3K inhibitor inhibits one or more activities of PI3K. In some embodiments, the anti-cancer therapy/PI3K inhibitor is (a) a small molecule that inhibits one or more enzymatic activities of PI3K, (b) an antibody that inhibits one or more activities of PI3K (e.g., by binding to and inhibiting one or more activities of PI3K, binding to and inhibiting expression of PI3K, and/or binding to and inhibiting one or more activities of a cell expressing PI3K, such as by inducing antibody-dependent cellular cytotoxicity, ADCC, or phagocytosis, ADCP), or (c) a nucleic acid that inhibits expression of PI3K (e.g., an antisense oligonucleotide, miRNA, siRNA, morpholino, CRISPR-based therapeutic, and the like). In some embodiments, the PI3K inhibitor is a small molecule inhibitor of PI3K (e.g., a competitive or non-competitive inhibitor). Non-limiting examples of PI3K inhibitors include GSK2636771, buparlisib (BKM120), AZD8186, copanlisib (BAY80-6946), LY294002, PX-866, TGX115, TGX126, BEZ235, SF1126, idelalisib (GS-1101, CAL-101), pictilisib (GDC-094), GDC0032, IPI145, INK1117 (MLN1117), SAR260301, KIN-193 (AZD6482), duvelisib, GS-9820, GSK2636771, GDC-0980, AMG319, pazobanib, and alpelisib (BYL719, Piqray), as well as pharmaceutically acceptable salts thereof. In some embodiments, the AKT inhibitor inhibits one or more activities of AKT (e.g., AKT1). In some embodiments, the anti-cancer therapy/AKT inhibitor is (a) a small molecule that inhibits one or more enzymatic activities of AKT1, (b) an antibody that inhibits one or more activities of AKT1 (e.g., by binding to and inhibiting one or more activities of AKT1, binding to and inhibiting expression of AKT1, and/or binding to and inhibiting one or more activities of a cell expressing AKT1, such as by inducing antibody-dependent cellular cytotoxicity, ADCC, or phagocytosis, ADCP), or (c) a nucleic acid that inhibits expression of AKT1 (e.g., an antisense oligonucleotide, miRNA, siRNA, morpholino, CRISPR-based therapeutic, and the like). In some embodiments, the AKT1 inhibitor is a small molecule inhibitor of AKT1 (e.g., a competitive or non-competitive inhibitor). Non-limiting examples of AKT1 inhibitors include GSK690693, GSK2141795 (uprosertib), GSK2110183 (afuresertib), AZD5363, GDC-0068 (ipatasertib), AT7867, CCT128930, MK-2206, BAY 1125976, AKT1 and AKT2-IN-1, perifosine, and VIII, as well as pharmaceutically acceptable salts thereof. In some embodiments, the AKT1 inhibitor is a pan-Akt inhibitor.

In some embodiments, the anti-cancer therapy is a hedgehog (Hh) inhibitor. In some embodiments, the anti-cancer therapy/Hh inhibitor is (a) a small molecule that inhibits one or more enzymatic activities of Hh, (b) an antibody that inhibits one or more activities of Hh (e.g., by binding to and inhibiting one or more activities of Hh, binding to and inhibiting expression of Hh, and/or binding to and inhibiting one or more activities of a cell expressing Hh, such as by inducing antibody-dependent cellular cytotoxicity, ADCC, or phagocytosis, ADCP), or (c) a nucleic acid that inhibits expression of Hh (e.g., an antisense oligonucleotide, miRNA, siRNA, morpholino, CRISPR-based therapeutic, and the like). In some embodiments, the Hh inhibitor is a small molecule inhibitor of Hh (e.g., a competitive or non-competitive inhibitor). Non-limiting examples of Hh inhibitors include sonidegib, vismodegib, erismodegib, saridegib, BMS833923, PF-04449913, and LY2940680, as well as pharmaceutically acceptable salts thereof.

In some embodiments, the anti-cancer therapy comprises a heat shock protein (HSP) inhibitor, a MYC inhibitor, an HDAC inhibitor, an immunotherapy, a neoantigen, a vaccine, or a cellular therapy.

In some embodiments, the anti-cancer therapy comprises one or more of an immune checkpoint inhibitor, a chemotherapy, a VEGF inhibitor, an Integrin 33 inhibitor, a statin, an EGFR inhibitor, an mTOR inhibitor, a PI3K inhibitor, a MAPK inhibitor, or a CDK4/6 inhibitor.

In some embodiments, the anti-cancer therapy comprises a kinase inhibitor. In some embodiments, the methods provided herein comprise administering to the individual a kinase inhibitor, e.g., in combination with another anti-cancer therapy. In some embodiments, the kinase inhibitor is crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, or TAE684 (NVP-TAE684). In some embodiments, the kinase inhibitor is an ALK kinase inhibitor, e.g., as described in examples 3-39 of WO2005016894, which is incorporated herein by reference.

In some embodiments, the anti-cancer therapy comprises a heat shock protein (HSP) inhibitor. In some embodiments, the methods provided herein comprise administering to the individual an HSP inhibitor, e.g., in combination with another anti-cancer therapy. In some embodiments, the HSP inhibitor is a Pan-HSP inhibitor, such as KNK423. In some embodiments, the HSP inhibitor is an HSP70 inhibitor, such as cmHsp70.1, quercetin, VER155008, or 17-AAD. In some embodiments, the HSP inhibitor is a HSP90 inhibitor. In some embodiments, the HSP90 inhibitor is 17-AAD, Debio0932, ganetespib (STA-9090), retaspimycin hydrochloride (retaspimycin, IPI-504), AUY922, alvespimycin (KOS-1022, 17-DMAG), tanespimycin (KOS-953, 17-AAG), DS 2248, or AT13387 (onalespib). In some embodiments, the HSP inhibitor is an HSP27 inhibitor, such as Apatorsen (OGX-427).

In some embodiments, the anti-cancer therapy comprises a MYC inhibitor. In some embodiments, the methods provided herein comprise administering to the individual a MYC inhibitor, e.g., in combination with another anti-cancer therapy. In some embodiments, the MYC inhibitor is MYCi361 (NUCC-0196361), MYCi975 (NUCC-0200975), Omomyc (dominant negative peptide), ZINC16293153 (Min9), 10058-F4, JKY-2-169, 7594-0035, or inhibitors of MYC/MAX dimerization and/or MYC/MAX/DNA complex formation.

In some embodiments, the anti-cancer therapy comprises a histone deacetylase (HDAC) inhibitor. In some embodiments, the methods provided herein comprise administering to the individual an HDAC inhibitor, e.g., in combination with another anti-cancer therapy. In some embodiments, the HDAC inhibitor is belinostat (PXD101, Beleodaq®), SAHA (vorinostat, suberoylanilide hydroxamine, Zolinza®), panobinostat (LBH589, LAQ-824), ACY1215 (Rocilinostat), quisinostat (JNJ-26481585), abexinostat (PCI-24781), pracinostat (SB939), givinostat (ITF2357), resminostat (4SC-201), trichostatin A (TSA), MS-275 (etinostat), Romidepsin (depsipeptide, FK228), MGCD0103 (mocetinostat), BML-210, CAY10603, valproic acid, MC1568, CUDC-907, CI-994 (Tacedinaline), Pivanex (AN-9), AR-42, Chidamide (CS055, HBI-8000), CUDC-101, CHR-3996, MPTOE028, BRD8430, MRLB-223, apicidin, RGFP966, BG45, PCI-34051, C149 (NCC149), TMP269, Cpd2, T247, T326, LMK235, CIA, HPOB, Nexturastat A, Befexamac, CBHA, Phenylbutyrate, MC1568, SNDX275, Scriptaid, Merck60, PX089344, PX105684, PX117735, PX117792, PX117245, PX105844, compound 12 as described by Li et al., Cold Spring Harb Perspect Med (2016) 6(10):a026831, or PX117445.

In some embodiments, the anti-cancer therapy comprises a VEGF inhibitor. In some embodiments, the methods provided herein comprise administering to the individual a VEGF inhibitor, e.g., in combination with another anti-cancer therapy. In some embodiments, the VEGF inhibitor is Bevacizumab (Avastin®), BMS-690514, ramucirumab, pazopanib, sorafenib, sunitinib, golvatinib, vandetanib, cabozantinib, levantinib, axitinib, cediranib, tivozanib, lucitanib, semaxanib, nindentanib, regorafinib, or aflibercept.

In some embodiments, the anti-cancer therapy comprises an integrin β3 inhibitor. In some embodiments, the methods provided herein comprise administering to the individual an integrin 03 inhibitor, e.g., in combination with another anti-cancer therapy. In some embodiments, the integrin β3 inhibitor is anti-avb3 (clone LM609), cilengitide (EMD121974, NSC, 707544), an siRNA, GLPG0187, MK-0429, CNTO95, TN-161, etaracizumab (MEDI-522), intetumumab (CNTO95) (anti-alphaV subunit antibody), abituzumab (EMD 525797/DI17E6) (anti-alphaV subunit antibody), JSM6427, SJ749, BCH-15046, SCH221153, or SC56631. In some embodiments, the anti-cancer therapy comprises an uIIbP3 integrin inhibitor. In some embodiments, the methods provided herein comprise administering to the individual an αIIbβ3 integrin inhibitor, e.g., in combination with another anti-cancer therapy. In some embodiments, the αIIbβ3 integrin inhibitor is abciximab, eptifibatide (Integrilin®), or tirofiban (Aggrastat®).

In some embodiments, the anti-cancer therapy comprises a statin or a statin-based agent. In some embodiments, the methods provided herein comprise administering to the individual a statin or a statin-based agent, e.g., in combination with another anti-cancer therapy. In some embodiments, the statin or statin-based agent is simvastatin, atorvastatin, fluvastatin, pitavastatin, pravastatin, rosuvastatin, or cerivastatin.

In some embodiments, the anti-cancer therapy comprises a MAPK inhibitor. In some embodiments, the methods provided herein comprise administering to the individual a MAPK inhibitor, e.g., in combination with another anti-cancer therapy. In some embodiments, the MAPK inhibitor is SB203580, SKF-86002, BIRB-796, SC-409, RJW-67657, BIRB-796, VX-745, R03201195, SB-242235, or MW181.

In some embodiments, the anti-cancer therapy comprises an EGFR inhibitor. In some embodiments, the methods provided herein comprise administering to the individual an EGFR inhibitor, e.g., in combination with another anti-cancer therapy. In some embodiments, the EGFR inhibitor is cetuximab, panitumumab, lapatinib, gefitinib, vandetanib, dacomitinib, icotinib, osimertinib (AZD9291), afatanib, olmutinib, EGF816 (nazartinib), avitinib (AC0010), rociletinib (CO-1686), BMS-690514, YH5448, PF-06747775, ASP8273, PF299804, AP26113, or erlotinib. In some embodiments, the EGFR inhibitor is gefitinib or cetuximab.

In some embodiments, the anti-cancer therapy comprises a cancer immunotherapy, such as a checkpoint inhibitor, cancer vaccine, cell-based therapy, T cell receptor (TCR)-based therapy, adjuvant immunotherapy, cytokine immunotherapy, and oncolytic virus therapy. In some embodiments, the methods provided herein comprise administering to the individual a cancer immunotherapy, such as a checkpoint inhibitor, cancer vaccine, cell-based therapy, T cell receptor (TCR)-based therapy, adjuvant immunotherapy, cytokine immunotherapy, and oncolytic virus therapy, e.g., in combination with another anti-cancer therapy. In some embodiments, the cancer immunotherapy comprises a small molecule, nucleic acid, polypeptide, carbohydrate, toxin, cell-based agent, or cell-binding agent. Examples of cancer immunotherapies are described in greater detail herein but are not intended to be limiting. In some embodiments, the cancer immunotherapy activates one or more aspects of the immune system to attack a cell (e.g., a tumor cell) that expresses a neoantigen, e.g., a neoantigen expressed by a cancer of the disclosure. The cancer immunotherapies of the present disclosure are contemplated for use as monotherapies, or in combination approaches comprising two or more in any combination or number, subject to medical judgement. Any of the cancer immunotherapies (optionally as monotherapies or in combination with another cancer immunotherapy or other therapeutic agent described herein) may find use in any of the methods described herein.

In some embodiments, the cancer immunotherapy comprises a cancer vaccine. A range of cancer vaccines have been tested that employ different approaches to promoting an immune response against a cancer (see, e.g., Emens L A, Expert Opin Emerg Drugs 13(2): 295-308 (2008) and US20190367613). Approaches have been designed to enhance the response of B cells, T cells, or professional antigen-presenting cells against tumors. Exemplary types of cancer vaccines include, but are not limited to, DNA-based vaccines, RNA-based vaccines, virus transduced vaccines, peptide-based vaccines, dendritic cell vaccines, oncolytic viruses, whole tumor cell vaccines, tumor antigen vaccines, etc. In some embodiments, the cancer vaccine can be prophylactic or therapeutic. In some embodiments, the cancer vaccine is formulated as a peptide-based vaccine, a nucleic acid-based vaccine, an antibody based vaccine, or a cell based vaccine. For example, a vaccine composition can include naked cDNA in cationic lipid formulations; lipopeptides (e.g., Vitiello, A. et ah, J. Clin. Invest. 95:341, 1995), naked cDNA or peptides, encapsulated e.g., in poly(DL-lactide-co-glycolide) (“PLG”) microspheres (see, e.g., Eldridge, et ah, Molec. Immunol. 28:287-294, 1991: Alonso et al, Vaccine 12:299-306, 1994; Jones et al, Vaccine 13:675-681, 1995); peptide composition contained in immune stimulating complexes (ISCOMS) (e.g., Takahashi et al, Nature 344:873-875, 1990; Hu et al, Clin. Exp. Immunol. 113:235-243, 1998); or multiple antigen peptide systems (MAPs) (see e.g., Tam, J. P., Proc. Natl Acad. Sci. U.S.A. 85:5409-5413, 1988; Tam, J. P., J. Immunol. Methods 196: 17-32, 1996). In some embodiments, a cancer vaccine is formulated as a peptide-based vaccine, or nucleic acid based vaccine in which the nucleic acid encodes the polypeptides. In some embodiments, a cancer vaccine is formulated as an antibody-based vaccine. In some embodiments, a cancer vaccine is formulated as a cell based vaccine. In some embodiments, the cancer vaccine is a peptide cancer vaccine, which in some embodiments is a personalized peptide vaccine. In some embodiments, the cancer vaccine is a multivalent long peptide, a multiple peptide, a peptide mixture, a hybrid peptide, or a peptide pulsed dendritic cell vaccine (see, e.g., Yamada et al, Cancer Sci, 104: 14-21), 2013). In some embodiments, such cancer vaccines augment the anti-cancer response.

In some embodiments, the cancer vaccine comprises a polynucleotide that encodes a neoantigen, e.g., a neoantigen expressed by a cancer of the disclosure. In some embodiments, the cancer vaccine comprises DNA that encodes a neoantigen, e.g., a neoantigen expressed by a cancer of the disclosure. In some embodiments, the cancer vaccine comprises RNA that encodes a neoantigen, e.g., a neoantigen expressed by a cancer of the disclosure. In some embodiments, the cancer vaccine comprises a polynucleotide that encodes a neoantigen, e.g., a neoantigen expressed by a cancer of the disclosure. In some embodiments, the cancer vaccine further comprises one or more additional antigens, neoantigens, or other sequences that promote antigen presentation and/or an immune response. In some embodiments, the polynucleotide is complexed with one or more additional agents, such as a liposome or lipoplex. In some embodiments, the polynucleotide(s) are taken up and translated by antigen presenting cells (APCs), which then present the neoantigen(s) via MHC class I on the APC cell surface.

In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, the cancer vaccine is selected from an oncolytic viral therapy such as pexastimogene devacirepvec (PexaVec/JX-594, SillaJen/formerly Jennerex Biotherapeutics), a thymidine kinase− (TK−) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543). prostate cancer (NCT01619813), head and neck squamous cell cancer (NCT01166542), pancreatic adenocarcinoma (NCT00998322), and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117), metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676), and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260), fallopian tube cancer, ovarian cancer (NCT 02759588); or CGO070 (Cold Genesys), an adenovirus engineered to express GM-CSF in bladder cancer (NCT02365818); anti-gp100; STINGVAX; GVAX; DCVaxL; and DNX-2401. In some embodiments, the cancer vaccine is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TGO1 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFu-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8+ T cell response. In some embodiments, the cancer vaccine comprises a vector-based tumor antigen vaccine. Vector-based tumor antigen vaccines can be used as a way to provide a steady supply of antigens to stimulate an anti-tumor immune response. In some embodiments, vectors encoding for tumor antigens are injected into an individual (possibly with pro-inflammatory or other attractants such as GM-CSF), taken up by cells in vivo to make the specific antigens, which then provoke the desired immune response. In some embodiments, vectors may be used to deliver more than one tumor antigen at a time, to increase the immune response. In addition, recombinant virus, bacteria or yeast vectors can trigger their own immune responses, which may also enhance the overall immune response.

In some embodiments, the cancer vaccine comprises a DNA-based vaccine. In some embodiments, DNA-based vaccines can be employed to stimulate an anti-tumor response. The ability of directly injected DNA that encodes an antigenic protein, to elicit a protective immune response has been demonstrated in numerous experimental systems. Vaccination through directly injecting DNA that encodes an antigenic protein, to elicit a protective immune response often produces both cell-mediated and humoral responses. Moreover, reproducible immune responses to DNA encoding various antigens have been reported in mice that last essentially for the lifetime of the animal (see, e.g., Yankauckas et al. (1993) DNA Cell Biol., 12: 771-776). In some embodiments, plasmid (or other vector) DNA that includes a sequence encoding a protein operably linked to regulatory elements required for gene expression is administered to individuals (e.g. human patients, non-human mammals, etc.). In some embodiments, the cells of the individual take up the administered DNA and the coding sequence is expressed. In some embodiments, the antigen so produced becomes a target against which an immune response is directed.

In some embodiments, the cancer vaccine comprises an RNA-based vaccine. In some embodiments, RNA-based vaccines can be employed to stimulate an anti-tumor response. In some embodiments, RNA-based vaccines comprise a self-replicating RNA molecule. In some embodiments, the self-replicating RNA molecule may be an alphavirus-derived RNA replicon. Self-replicating RNA (or “SAM”) molecules are well known in the art and can be produced by using replication elements derived from, e.g., alphaviruses, and substituting the structural viral proteins with a nucleotide sequence encoding a protein of interest. A self-replicating RNA molecule is typically a +−strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the antigen.

In some embodiments, the cancer immunotherapy comprises a cell-based therapy. In some embodiments, the cancer immunotherapy comprises a T cell-based therapy. In some embodiments, the cancer immunotherapy comprises an adoptive therapy, e.g., an adoptive T cell-based therapy. In some embodiments, the T cells are autologous or allogeneic to the recipient. In some embodiments, the T cells are CD8+ T cells. In some embodiments, the T cells are CD4+ T cells. Adoptive immunotherapy refers to a therapeutic approach for treating cancer or infectious diseases in which immune cells are administered to a host with the aim that the cells mediate either directly or indirectly specific immunity to (i.e., mount an immune response directed against) cancer cells. In some embodiments, the immune response results in inhibition of tumor and/or metastatic cell growth and/or proliferation, and in related embodiments, results in neoplastic cell death and/or resorption. The immune cells can be derived from a different organism/host (exogenous immune cells) or can be cells obtained from the subject organism (autologous immune cells). In some embodiments, the immune cells (e.g., autologous or allogeneic T cells (e.g., regulatory T cells, CD4+ T cells, CD8+ T cells, or gamma-delta T cells), NK cells, invariant NK cells, or NKT cells) can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In some embodiments, NK cells are engineered to express a TCR. The NK cells may be further engineered to express a CAR. Multiple CARs and/or TCRs, such as to different antigens, may be added to a single cell type, such as T cells or NK cells. In some embodiments, the cells comprise one or more nucleic acids/expression constructs/vectors introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g. chimeric). In some embodiments, a population of immune cells can be obtained from a subject in need of therapy or suffering from a disease associated with reduced immune cell activity. Thus, the cells will be autologous to the subject in need of therapy. In some embodiments, a population of immune cells can be obtained from a donor, such as a histocompatibility-matched donor. In some embodiments, the immune cell population can be harvested from the peripheral blood, cord blood, bone marrow, spleen, or any other organ/tissue in which immune cells reside in said subject or donor. In some embodiments, the immune cells can be isolated from a pool of subjects and/or donors, such as from pooled cord blood. In some embodiments, when the population of immune cells is obtained from a donor distinct from the subject, the donor may be allogeneic, provided the cells obtained are subject-compatible, in that they can be introduced into the subject. In some embodiments, allogeneic donor cells may or may not be human-leukocyte-antigen (HLA)-compatible. In some embodiments, to be rendered subject-compatible, allogeneic cells can be treated to reduce immunogenicity.

In some embodiments, the cell-based therapy comprises a T cell-based therapy, such as autologous cells, e.g., tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. Several approaches for the isolation, derivation, engineering or modification, activation, and expansion of functional anti-tumor effector cells have been described in the last two decades and may be used according to any of the methods provided herein. In some embodiments, the T cells are derived from the blood, bone marrow, lymph, umbilical cord, or lymphoid organs. In some embodiments, the cells are human cells. In some embodiments, the cells are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the cells may be allogeneic and/or autologous. In some embodiments, such as for off-the-shelf technologies, the cells are pluripotent and/or multipotent, such as stem cells, such as induced pluripotent stem cells (iPSCs).

In some embodiments, the T cell-based therapy comprises a chimeric antigen receptor (CAR)-T cell-based therapy. This approach involves engineering a CAR that specifically binds to an antigen of interest and comprises one or more intracellular signaling domains for T cell activation. The CAR is then expressed on the surface of engineered T cells (CAR-T) and administered to a patient, leading to a T-cell-specific immune response against cancer cells expressing the antigen. In some embodiments, the CAR specifically binds a neoantigen.

In some embodiments, the T cell-based therapy comprises T cells expressing a recombinant T cell receptor (TCR). This approach involves identifying a TCR that specifically binds to an antigen of interest, which is then used to replace the endogenous or native TCR on the surface of engineered T cells that are administered to a patient, leading to a T-cell-specific immune response against cancer cells expressing the antigen. In some embodiments, the recombinant TCR specifically binds a neoantigen.

In some embodiments, the T cell-based therapy comprises tumor-infiltrating lymphocytes (TILs). For example, TILs can be isolated from a tumor or cancer of the present disclosure, then isolated and expanded in vitro. Some or all of these TILs may specifically recognize an antigen expressed by the tumor or cancer of the present disclosure. In some embodiments, the TILs are exposed to one or more neoantigens, e.g., a neoantigen, in vitro after isolation. TILs are then administered to the patient (optionally in combination with one or more cytokines or other immune-stimulating substances).

In some embodiments, the cell-based therapy comprises a natural killer (NK) cell-based therapy. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells can be detected by specific surface markers, such as CD16, CD56, and CD8 in humans. NK cells do not express T-cell antigen receptors, the pan T marker CD3, or surface immunoglobulin B cell receptors. In some embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art.

In some embodiments, the cell-based therapy comprises a dendritic cell (DC)-based therapy, e.g., a dendritic cell vaccine. In some embodiments, the DC vaccine comprises antigen-presenting cells that are able to induce specific T cell immunity, which are harvested from the patient or from a donor. In some embodiments, the DC vaccine can then be exposed in vitro to a peptide antigen, for which T cells are to be generated in the patient. In some embodiments, dendritic cells loaded with the antigen are then injected back into the patient. In some embodiments, immunization may be repeated multiple times if desired. Methods for harvesting, expanding, and administering dendritic cells are known in the art; see, e.g., WO2019178081. Dendritic cell vaccines (such as Sipuleucel-T, also known as APC8015 and PROVENGE®) are vaccines that involve administration of dendritic cells that act as APCs to present one or more cancer-specific antigens to the patient's immune system. In some embodiments, the dendritic cells are autologous or allogeneic to the recipient.

In some embodiments, the cancer immunotherapy comprises a TCR-based therapy. In some embodiments, the cancer immunotherapy comprises administration of one or more TCRs or TCR-based therapeutics that specifically bind an antigen expressed by a cancer of the present disclosure. In some embodiments, the TCR-based therapeutic may further include a moiety that binds an immune cell (e.g., a T cell), such as an antibody or antibody fragment that specifically binds a T cell surface protein or receptor (e.g., an anti-CD3 antibody or antibody fragment).

In some embodiments, the immunotherapy comprises adjuvant immunotherapy. Adjuvant immunotherapy comprises the use of one or more agents that activate components of the innate immune system, e.g., HILTONOL® (imiquimod), which targets the TLR7 pathway.

In some embodiments, the immunotherapy comprises cytokine immunotherapy. Cytokine immunotherapy comprises the use of one or more cytokines that activate components of the immune system. Examples include, but are not limited to, aldesleukin (PROLEUKIN®; interleukin-2), interferon alfa-2a (ROFERON®-A), interferon alfa-2b (INTRON®-A), and peginterferon alfa-2b (PEGINTRON®).

In some embodiments, the immunotherapy comprises oncolytic virus therapy. Oncolytic virus therapy uses genetically modified viruses to replicate in and kill cancer cells, leading to the release of antigens that stimulate an immune response. In some embodiments, replication-competent oncolytic viruses expressing a tumor antigen comprise any naturally occurring (e.g., from a “field source”) or modified replication-competent oncolytic virus. In some embodiments, the oncolytic virus, in addition to expressing a tumor antigen, may be modified to increase selectivity of the virus for cancer cells. In some embodiments, replication-competent oncolytic viruses include, but are not limited to, oncolytic viruses that are a member in the family of myoviridae, siphoviridae, podpviridae, teciviridae, corticoviridae, plasmaviridae, lipothrixviridae, fuselloviridae, poxyiridae, iridoviridae, phycodnaviridae, baculoviridae, herpesviridae, adnoviridae, papovaviridae, polydnaviridae, inoviridae, microviridae, geminiviridae, circoviridae, parvoviridae, hcpadnaviridae, retroviridae, cyctoviridae, reoviridae, birnaviridae, paramyxoviridae, rhabdoviridae, filoviridae, orthomyxoviridae, bunyaviridae, arenaviridae, Leviviridae, picornaviridae, sequiviridae, comoviridae, potyviridae, caliciviridae, astroviridae, nodaviridae, tetraviridae, tombusviridae, coronaviridae, glaviviridae, togaviridae, and barnaviridae. In some embodiments, replication-competent oncolytic viruses include adenovirus, retrovirus, reovirus, rhabdovirus, Newcastle Disease virus (NDV), polyoma virus, vaccinia virus (VacV), herpes simplex virus, picornavirus, coxsackie virus and parvovirus. In some embodiments, a replicative oncolytic vaccinia virus expressing a tumor antigen may be engineered to lack one or more functional genes in order to increase the cancer selectivity of the virus. In some embodiments, an oncolytic vaccinia virus is engineered to lack thymidine kinase (TK) activity. In some embodiments, the oncolytic vaccinia virus may be engineered to lack vaccinia virus growth factor (VGF). In some embodiments, an oncolytic vaccinia virus may be engineered to lack both VGF and TK activity. In some embodiments, an oncolytic vaccinia virus may be engineered to lack one or more genes involved in evading host interferon (IFN) response such as E3L, K3L, B18R, or B8R. In some embodiments, a replicative oncolytic vaccinia virus is a Western Reserve, Copenhagen, Lister or Wyeth strain and lacks a functional TK gene. In some embodiments, the oncolytic vaccinia virus is a Western Reserve, Copenhagen, Lister or Wyeth strain lacking a functional B18R and/or B8R gene. In some embodiments, a replicative oncolytic vaccinia virus expressing a tumor antigen may be locally or systemically administered to a subject, e.g. via intratumoral, intraperitoneal, intravenous, intra-arterial, intramuscular, intradermal, intracranial, subcutaneous, or intranasal administration.

In some embodiments, the anti-cancer therapy comprises an immune checkpoint inhibitor. In some embodiments, the methods provided herein comprise administering to the individual an immune checkpoint inhibitor, e.g., in combination with another anti-cancer therapy. In some embodiments, the methods provided herein comprise administering to an individual an effective amount of an immune checkpoint inhibitor. As is known in the art, a checkpoint inhibitor targets at least one immune checkpoint protein to alter the regulation of an immune response. Immune checkpoint proteins include, e.g., CTLA4, PD-L1, PD-1, PD-L2, VISTA, B7-H2, B7-H3, B7-H4, B7-H6, 2B4, ICOS, HVEM, CEACAM, LAIR1, CD80, CD86, CD276, VTCN1, MHC class I, MHC class II, GALS, adenosine, TGFR, CSF1R, MICA/B, arginase, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, LAG-3, BTLA, IDO, OX40, and A2aR. In some embodiments, molecules involved in regulating immune checkpoints include, but are not limited to: PD-1 (CD279), PD-L1 (B7-H1, CD274), PD-L2 (B7-CD, CD273), CTLA-4 (CD152), HVEM, BTLA (CD272), a killer-cell immunoglobulin-like receptor (KIR), LAG-3 (CD223), TIM-3 (HAVCR2), CEACAM, CEACAM-1, CEACAM-3, CEACAM-5, GAL9, VISTA (PD-1H), TIGIT, LAIR1, CD160, 2B4, TGFRbeta, A2AR, GITR (CD357), CD80 (B7-1), CD86 (B7-2), CD276 (B7-H3), VTCNI (B7-H4), MHC class I, MHC class II, GALS, adenosine, TGFR, B7-Hi, OX40 (CD134), CD94 (KLRD1), CD137 (4-1BB), CD137L (4-1BBL), CD40, IDO, CSF1R, CD40L, CD47, CD70 (CD27L), CD226, HHLA2, ICOS (CD278), ICOSL (CD275), LIGHT (TNFSFi4, CD258), NKG2a, NKG2d, OX40L (CD134L), PVR (NECL5, CD155), SIRPa, MICA/B, and/or arginase. In some embodiments, an immune checkpoint inhibitor (i.e., a checkpoint inhibitor) decreases the activity of a checkpoint protein that negatively regulates immune cell function, e.g., in order to enhance T cell activation and/or an anti-cancer immune response. In other embodiments, a checkpoint inhibitor increases the activity of a checkpoint protein that positively regulates immune cell function, e.g., in order to enhance T cell activation and/or an anti-cancer immune response. In some embodiments, the checkpoint inhibitor is an antibody. Examples of checkpoint inhibitors include, without limitation, a PD-1 axis binding antagonist, a PD-L1 axis binding antagonist (e.g., an anti-PD-L1 antibody, e.g., atezolizumab (MPDL3280A)), an antagonist directed against a co-inhibitory molecule (e.g., a CTLA4 antagonist (e.g., an anti-CTLA4 antibody), a TIM-3 antagonist (e.g., an anti-TIM-3 antibody), or a LAG-3 antagonist (e.g., an anti-LAG-3 antibody)), or any combination thereof. In some embodiments, the immune checkpoint inhibitors comprise drugs such as small molecules, recombinant forms of ligand or receptors, or antibodies, such as human antibodies (see, e.g., International Patent Publication WO2015016718; Pardoll, Nat Rev Cancer, 12(4): 252-64, 2012; both incorporated herein by reference). In some embodiments, known inhibitors of immune checkpoint proteins or analogs thereof may be used, in particular chimerized, humanized or human forms of antibodies may be used.

In some embodiments, the checkpoint inhibitor is a PD-L1 axis binding antagonist, e.g., a PD-1 binding antagonist, a PD-L1 binding antagonist, or a PD-L2 binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1 LG1,” “CD274,” “B7-H,” and “PDL1.” An exemplary human PD-L1 is shown in UniProtKB/Swiss-Prot Accession No. Q9NZQ7.1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, PD-1, PD-L1, and PD-L2 are human PD-1, PD-L1 and PD-L2.

In some instances, the PD-1 binding antagonist is a molecule that inhibits the binding of PD-1 to its ligand binding partners. In a specific embodiment, the PD-1 ligand binding partners are PD-L1 and/or PD-L2. In another instance, a PD-L1 binding antagonist is a molecule that inhibits the binding of PD-L1 to its binding ligands. In a specific embodiment, PD-L1 binding partners are PD-1 and/or B7-1. In another instance, the PD-L2 binding antagonist is a molecule that inhibits the binding of PD-L2 to its ligand binding partners. In a specific embodiment, the PD-L2 binding ligand partner is PD-1. The antagonist may be an antibody, an antigen binding fragment thereof, an immunoadhesin, a fusion protein, or an oligopeptide. In some embodiments, the PD-1 binding antagonist is a small molecule, a nucleic acid, a polypeptide (e.g., antibody), a carbohydrate, a lipid, a metal, or a toxin.

In some instances, the PD-1 binding antagonist is an anti-PD-1 antibody (e.g., a human antibody, a humanized antibody, or a chimeric antibody), for example, as described below. In some instances, the anti-PD-1 antibody is one or more of MDX-1 106 (nivolumab), MK-3475 (pembrolizumab, Keytruda®), MEDI-0680 (AMP-514), PDR001, REGN2810, MGA-012, JNJ-63723283, BI 754091, or BGB-108. In other instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some instances, the PD-1 binding antagonist is AMP-224. Other examples of anti-PD-1 antibodies include, but are not limited to, MEDI-0680 (AMP-514; AstraZeneca), PDR001 (CAS Registry No. 1859072-53-9; Novartis), REGN2810 (LIBTAYO® or cemiplimab-rwlc; Regeneron), BGB-108 (BeiGene), BGB-A317 (BeiGene), BI 754091, JS-001 (Shanghai Junshi), STI-A1110 (Sorrento), INCSHR-1210 (Incyte), PF-06801591 (Pfizer), TSR-042 (also known as ANB011; Tesaro/AnaptysBio), AM0001 (ARMO Biosciences), ENUM 244C8 (Enumeral Biomedical Holdings), or ENUM 388D4 (Enumeral Biomedical Holdings). In some embodiments, the PD-1 axis binding antagonist comprises tislelizumab (BGB-A317), BGB-108, STI-A1110, AM0001, BI 754091, sintilimab (IB1I308), cetrelimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), pembrolizumab (MK-3475, SCH 900475, Keytruda®), PF-06801591, cemiplimab (REGN-2810, REGEN2810), dostarlimab (TSR-042, ANB011), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-001, BY-24.3, CB-201, IBI-319, ONCR-177, Max-1, CS-4100, JBI-426, CCC-0701, or CCX-4503, or derivatives thereof.

In some embodiments, the PD-L1 binding antagonist is a small molecule that inhibits PD-1. In some embodiments, the PD-L1 binding antagonist is a small molecule that inhibits PD-L1. In some embodiments, the PD-L1 binding antagonist is a small molecule that inhibits PD-L1 and VISTA or PD-L1 and TIM3. In some embodiments, the PD-L1 binding antagonist is CA-170 (also known as AUPM-170). In some embodiments, the PD-L1 binding antagonist is an anti-PD-L1 antibody. In some embodiments, the anti-PD-L1 antibody can bind to a human PD-L1, for example a human PD-L1 as shown in UniProtKB/Swiss-Prot Accession No. Q9NZQ7.1, or a variant thereof. In some embodiments, the PD-L1 binding antagonist is a small molecule, a nucleic acid, a polypeptide (e.g., antibody), a carbohydrate, a lipid, a metal, or a toxin.

In some instances, the PD-L1 binding antagonist is an anti-PD-L1 antibody, for example, as described below. In some instances, the anti-PD-L1 antibody is capable of inhibiting the binding between PD-L1 and PD-1, and/or between PD-L1 and B7-1. In some instances, the anti-PD-L1 antibody is a monoclonal antibody. In some instances, the anti-PD-L1 antibody is an antibody fragment selected from a Fab, Fab′-SH, Fv, scFv, or (Fab′)2 fragment. In some instances, the anti-PD-L1 antibody is a humanized antibody. In some instances, the anti-PD-L1 antibody is a human antibody. In some instances, the anti-PD-L1 antibody is selected from YW243.55.S70, MPDL3280A (atezolizumab), MDX-1 105, MEDI4736 (durvalumab), or MSB0010718C (avelumab). In some embodiments, the PD-L1 axis binding antagonist comprises atezolizumab, avelumab, durvalumab (imfinzi), BGB-A333, SHR-1316 (HTI-1088), CK-301, BMS-936559, envafolimab (KN035, ASC22), CS1001, MDX-1105 (BMS-936559), LY3300054, STI-A1014, FAZ053, CX-072, INCB086550, GNS-1480, CA-170, CK-301, M-7824, HTI-1088 (HTI-131, SHR-1316), MSB-2311, AK-106, AVA-004, BBI-801, CA-327, CBA-0710, CBT-502, FPT-155, IKT-201, IKT-703, 10-103, JS-003, KD-033, KY-1003, MCLA-145, MT-5050, SNA-02, BCD-135, APL-502 (CBT-402 or TQB2450), IMC-001, KD-045, INBRX-105, KN-046, IMC-2102, IMC-2101, KD-005, IMM-2502, 89Zr-CX-072, 89Zr-DFO-6E11, KY-1055, MEDI-1109, MT-5594, SL-279252, DSP-106, Gensci-047, REMD-290, N-809, PRS-344, FS-222, GEN-1046, BH-29xx, or FS-118, or a derivative thereof.

In some embodiments, the checkpoint inhibitor is an antagonist of CTLA4. In some embodiments, the checkpoint inhibitor is a small molecule antagonist of CTLA4. In some embodiments, the checkpoint inhibitor is an anti-CTLA4 antibody. CTLA4 is part of the CD28-B7 immunoglobulin superfamily of immune checkpoint molecules that acts to negatively regulate T cell activation, particularly CD28-dependent T cell responses. CTLA4 competes for binding to common ligands with CD28, such as CD80 (B7-1) and CD86 (B7-2), and binds to these ligands with higher affinity than CD28. Blocking CTLA4 activity (e.g., using an anti-CTLA4 antibody) is thought to enhance CD28-mediated costimulation (leading to increased T cell activation/priming), affect T cell development, and/or deplete Tregs (such as intratumoral Tregs). In some embodiments, the CTLA4 antagonist is a small molecule, a nucleic acid, a polypeptide (e.g., antibody), a carbohydrate, a lipid, a metal, or a toxin. In some embodiments, the CTLA-4 inhibitor comprises ipilimumab (IB1I310, BMS-734016, MDX010, MDX-CTLA4, MEDI4736), tremelimumab (CP-675, CP-675,206), APL-509, AGEN1884, CS1002, AGEN1181, Abatacept (Orencia, BMS-188667, RG2077), BCD-145, ONC-392, ADU-1604, REGN4659, ADG116, KN044, KN046, or a derivative thereof.

In some embodiments, the anti-PD-1 antibody or antibody fragment is MDX-1106 (nivolumab), MK-3475 (pembrolizumab, Keytruda®), MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, or ENUM 388D4. In some embodiments, the PD-1 binding antagonist is an anti-PD-1 immunoadhesin. In some embodiments, the anti-PD-1 immunoadhesin is AMP-224. In some embodiments, the anti-PD-L1 antibody or antibody fragment is YW243.55.S70, MPDL3280A (atezolizumab), MDX-1105, MEDI4736 (durvalumab), MSB0010718C (avelumab), LY3300054, STI-A1014, KN035, FAZ053, or CX-072.

In some embodiments, the immune checkpoint inhibitor comprises a LAG-3 inhibitor (e.g., an antibody, an antibody conjugate, or an antigen-binding fragment thereof). In some embodiments, the LAG-3 inhibitor comprises a small molecule, a nucleic acid, a polypeptide (e.g., an antibody), a carbohydrate, a lipid, a metal, or a toxin. In some embodiments, the LAG-3 inhibitor comprises a small molecule. In some embodiments, the LAG-3 inhibitor comprises a LAG-3 binding agent. In some embodiments, the LAG-3 inhibitor comprises an antibody, an antibody conjugate, or an antigen-binding fragment thereof. In some embodiments, the LAG-3 inhibitor comprises eftilagimod alpha (IMP321, IMP-321, EDDP-202, EOC-202), relatlimab (BMS-986016), GSK2831781 (IMP-731), LAG525 (IMP701), TSR-033, EVIP321 (soluble LAG-3 protein), BI 754111, IMP761, REGN3767, MK-4280, MGD-013, XmAb22841, INCAGN-2385, ENUM-006, AVA-017, AM-0003, iOnctura anti-LAG-3 antibody, Arcus Biosciences LAG-3 antibody, Sym022, a derivative thereof, or an antibody that competes with any of the preceding.

In some embodiments, the anti-cancer therapy comprises an immunoregulatory molecule or a cytokine. In some embodiments, the methods provided herein comprise administering to the individual an immunoregulatory molecule or a cytokine, e.g., in combination with another anti-cancer therapy. An immunoregulatory profile is required to trigger an efficient immune response and balance the immunity in a subject. Examples of suitable immunoregulatory cytokines include, but are not limited to, interferons (e.g., IFNα, IFNβ and IFNγ), interleukins (e.g., IL-1, IL-2, IL-3, IL-4, IL-5, IL-6, IL-7, IL-8, IL-9, IL-10, IL-12 and IL-20), tumor necrosis factors (e.g., TNFα and TNFβ), erythropoietin (EPO), FLT-3 ligand, gIp10, TCA-3, MCP-1, MIF, MIP-1α, MIP-1β, Rantes, macrophage colony stimulating factor (M-CSF), granulocyte colony stimulating factor (G-CSF), or granulocyte-macrophage colony stimulating factor (GM-CSF), as well as functional fragments thereof. In some embodiments, any immunomodulatory chemokine that binds to a chemokine receptor, i.e., a CXC, CC, C, or CX3C chemokine receptor, can be used in the context of the present disclosure. Examples of chemokines include, but are not limited to, MIP-3α (Lax), MIP-3β, Hcc-1, MPIF-1, MPIF-2, MCP-2, MCP-3, MCP-4, MCP-5, Eotaxin, Tarc, Elc, 1309, IL-8, GCP-2 Groα, Gro-β, Nap-2, Ena-78, Ip-10, MIG, I-Tac, SDF-1, or BCA-1 (Blc), as well as functional fragments thereof. In some embodiments, the immunoregulatory molecule is included with any of the treatments provided herein.

In some embodiments, the immune checkpoint inhibitor is monovalent and/or monospecific. In some embodiments, the immune checkpoint inhibitor is multivalent and/or multispecific.

In some embodiments, the anti-cancer therapy comprises a nucleic acid molecule, such as a dsRNA, an siRNA, or an shRNA. In some embodiments, the methods provided herein comprise administering to the individual a nucleic acid molecule, such as a dsRNA, an siRNA, or an shRNA, e.g., in combination with another anti-cancer therapy. As is known in the art, dsRNAs having a duplex structure are effective at inducing RNA interference (RNAi). In some embodiments, the anti-cancer therapy comprises a small interfering RNA molecule (siRNA). dsRNAs and siRNAs can be used to silence gene expression in mammalian cells (e.g., human cells). In some embodiments, a dsRNA of the disclosure comprises any of between about 5 and about 10 base pairs, between about 10 and about 12 base pairs, between about 12 and about 15 base pairs, between about 15 and about 20 base pairs, between about 20 and 23 base pairs, between about 23 and about 25 base pairs, between about 25 and about 27 base pairs, or between about 27 and about 30 base pairs. As is known in the art, siRNAs are small dsRNAs that optionally include overhangs. In some embodiments, the duplex region of an siRNA is between about 18 and 25 nucleotides, e.g., any of 18, 19, 20, 21, 22, 23, 24, or 25 nucleotides. siRNAs may also include short hairpin RNAs (shRNAs), e.g., with approximately 29-base-pair stems and 2-nucleotide 3′ overhangs. Methods for designing, optimizing, producing, and using dsRNAs, siRNAs, or shRNAs, are known in the art.

In some embodiments, the anti-cancer therapy comprises a chemotherapy. In some embodiments, the methods provided herein comprise administering to the individual a chemotherapy, e.g., in combination with another anti-cancer therapy. Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenishers such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO); retinoids, such as retinoic acid; capecitabine; carboplatin, procarbazine, plicomycin, gemcitabine, navelbine, famesyl-protein tansferase inhibitors, transplatinum, and pharmaceutically acceptable salts, acids, or derivatives of any of the above.

Some non-limiting examples of chemotherapeutic drugs which can be combined with anti-cancer therapies of the present disclosure are carboplatin (Paraplatin), cisplatin (Platinol, Platinol-AQ), cyclophosphamide (Cytoxan, Neosar), docetaxel (Taxotere), doxorubicin (Adriamycin), erlotinib (Tarceva), etoposide (VePesid), fluorouracil (5-FU), gemcitabine (Gemzar), imatinib mesylate (Gleevec), irinotecan (Camptosar), methotrexate (Folex, Mexate, Amethopterin), paclitaxel (Taxol, Abraxane), sorafinib (Nexavar), sunitinib (Sutent), topotecan (Hycamtin), vincristine (Oncovin, Vincasar PFS), and vinblastine (Velban).

In some embodiments, the anti-cancer therapy comprises a kinase inhibitor. In some embodiments, the methods provided herein comprise administering to the individual a kinase inhibitor, e.g., in combination with another anti-cancer therapy. Examples of kinase inhibitors include those that target one or more receptor tyrosine kinases, e.g., BCR-ABL, B-Raf, EGFR, HER-2/ErbB2, IGF-IR, PDGFR-a, PDGFR-β, cKit, Flt-4, Flt3, FGFR1, FGFR3, FGFR4, CSF1R, c-Met, RON, c-Ret, or ALK; one or more cytoplasmic tyrosine kinases, e.g., c-SRC, c-YES, Abl, or JAK-2; one or more serine/threonine kinases, e.g., ATM, Aurora A & B, CDKs, mTOR, PKCi, PLKs, b-Raf, S6K, or STK11/LKB1; or one or more lipid kinases, e.g., PI3K or SKI. Small molecule kinase inhibitors include PHA-739358, nilotinib, dasatinib, PD166326, NSC 743411, lapatinib (GW-572016), canertinib (CI-1033), semaxinib (SU5416), vatalanib (PTK787/ZK222584), sutent (SU1 1248), sorafenib (BAY 43-9006), or leflunomide (SU101). Additional non-limiting examples of tyrosine kinase inhibitors include imatinib (Gleevec/Glivec) and gefitinib (Iressa).

In some embodiments, the anti-cancer therapy comprises an anti-angiogenic agent. In some embodiments, the methods provided herein comprise administering to the individual an anti-angiogenic agent, e.g., in combination with another anti-cancer therapy. Angiogenesis inhibitors prevent the extensive growth of blood vessels (angiogenesis) that tumors require to survive. Non-limiting examples of angiogenesis-mediating molecules or angiogenesis inhibitors which may be used in the methods of the present disclosure include soluble VEGF (for example: VEGF isoforms, e.g., VEGF121 and VEGF165; VEGF receptors, e.g., VEGFR1, VEGFR2; and co-receptors, e.g., Neuropilin-1 and Neuropilin-2), NRP-1, angiopoietin 2, TSP-1 and TSP-2, angiostatin and related molecules, endostatin, vasostatin, calreticulin, platelet factor-4, TIMP and CDAI, Meth-1 and Meth-2, IFNα, IFN-β and IFN-γ, CXCL10, IL-4, IL-12 and IL-18, prothrombin (kringle domain-2), antithrombin III fragment, prolactin, VEGI, SPARC, osteopontin, maspin, canstatin, proliferin-related protein, restin and drugs such as bevacizumab, itraconazole, carboxyamidotriazole, TNP-470, CM101, IFN-α platelet factor-4, suramin, SU5416, thrombospondin, VEGFR antagonists, angiostatic steroids and heparin, cartilage-derived angiogenesis inhibitory factor, matrix metalloproteinase inhibitors, 2-methoxyestradiol, tecogalan, tetrathiomolybdate, thalidomide, thrombospondin, prolactina v β3 inhibitors, linomide, or tasquinimod. In some embodiments, known therapeutic candidates that may be used according to the methods of the disclosure include naturally occurring angiogenic inhibitors, including without limitation, angiostatin, endostatin, or platelet factor-4. In another embodiment, therapeutic candidates that may be used according to the methods of the disclosure include, without limitation, specific inhibitors of endothelial cell growth, such as TNP-470, thalidomide, and interleukin-12. Still other anti-angiogenic agents that may be used according to the methods of the disclosure include those that neutralize angiogenic molecules, including without limitation, antibodies to fibroblast growth factor, antibodies to vascular endothelial growth factor, antibodies to platelet derived growth factor, or antibodies or other types of inhibitors of the receptors of EGF, VEGF or PDGF. In some embodiments, anti-angiogenic agents that may be used according to the methods of the disclosure include, without limitation, suramin and its analogs, and tecogalan. In other embodiments, anti-angiogenic agents that may be used according to the methods of the disclosure include, without limitation, agents that neutralize receptors for angiogenic factors or agents that interfere with vascular basement membrane and extracellular matrix, including, without limitation, metalloprotease inhibitors and angiostatic steroids. Another group of anti-angiogenic compounds that may be used according to the methods of the disclosure includes, without limitation, anti-adhesion molecules, such as antibodies to integrin alpha v beta 3. Still other anti-angiogenic compounds or compositions that may be used according to the methods of the disclosure include, without limitation, kinase inhibitors, thalidomide, itraconazole, carboxyamidotriazole, CM101, IFN-α, IL-12, SU5416, thrombospondin, cartilage-derived angiogenesis inhibitory factor, 2-methoxyestradiol, tetrathiomolybdate, thrombospondin, prolactin, and linomide. In one particular embodiment, the anti-angiogenic compound that may be used according to the methods of the disclosure is an antibody to VEGF, such as Avastin®/bevacizumab (Genentech).

In some embodiments, the anti-cancer therapy comprises an anti-DNA repair therapy. In some embodiments, the methods provided herein comprise administering to the individual an anti-DNA repair therapy, e.g., in combination with another anti-cancer therapy. In some embodiments, the anti-DNA repair therapy is a PARP inhibitor (e.g., talazoparib, rucaparib, olaparib), a RAD51 inhibitor (e.g., RI-1), or an inhibitor of a DNA damage response kinase, e.g., CHCK1 (e.g., AZD7762), ATM (e.g., KU-55933, KU-60019, NU7026, or VE-821), and ATR (e.g., NU7026).

In some embodiments, the anti-cancer therapy comprises a radiosensitizer. In some embodiments, the methods provided herein comprise administering to the individual a radiosensitizer, e.g., in combination with another anti-cancer therapy. Exemplary radiosensitizers include hypoxia radiosensitizers such as misonidazole, metronidazole, and trans-sodium crocetinate, a compound that helps to increase the diffusion of oxygen into hypoxic tumor tissue. The radiosensitizer can also be a DNA damage response inhibitor interfering with base excision repair (BER), nucleotide excision repair (NER), mismatch repair (MMR), recombinational repair comprising homologous recombination (HR) and non-homologous end-joining (NHEJ), and direct repair mechanisms. Single strand break (SSB) repair mechanisms include BER, NER, or MMR pathways, while double stranded break (DSB) repair mechanisms consist of HR and NHEJ pathways. Radiation causes DNA breaks that, if not repaired, are lethal. SSBs are repaired through a combination of BER, NER and MMR mechanisms using the intact DNA strand as a template. The predominant pathway of SSB repair is BER, utilizing a family of related enzymes termed poly-(ADP-ribose) polymerases (PARP). Thus, the radiosensitizer can include DNA damage response inhibitors such as PARP inhibitors.

In some embodiments, the anti-cancer therapy comprises an anti-inflammatory agent. In some embodiments, the methods provided herein comprise administering to the individual an anti-inflammatory agent, e.g., in combination with another anti-cancer therapy. In some embodiments, the anti-inflammatory agent is an agent that blocks, inhibits, or reduces inflammation or signaling from an inflammatory signaling pathway In some embodiments, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23; interferons (IFNs), e.g., IFNα, IFNβ, IFNγ, IFN-γ inducing factor (IGIF); transforming growth factor-β (TGF-β); transforming growth factor-α (TGF-α); tumor necrosis factors, e.g., TNF-α, TNF-β, TNF-RI, TNF-RII; CD23; CD30; CD40L; EGF; G-CSF; GDNF; PDGF-BB; RANTES/CCL5; IKK; NF-κB; TLR2; TLR3; TLR4; TL5; TLR6; TLR7; TLR8; TLR8; TLR9; and/or any cognate receptors thereof. In some embodiments, the anti-inflammatory agent is an IL-1 or IL-1 receptor antagonist, such as anakinra (Kineret®), rilonacept, or canakinumab. In some embodiments, the anti-inflammatory agent is an IL-6 or IL-6 receptor antagonist, e.g., an anti-IL-6 antibody or an anti-IL-6 receptor antibody, such as tocilizumab (ACTEMRA®), olokizumab, clazakizumab, sarilumab, sirukumab, siltuximab, or ALX-0061. In some embodiments, the anti-inflammatory agent is a TNF-α antagonist, e.g., an anti-TNFα antibody, such as infliximab (Remicade®), golimumab (Simponi®), adalimumab (Humira®), certolizumab pegol (Cimzia®) or etanercept. In some embodiments, the anti-inflammatory agent is a corticosteroid. Exemplary corticosteroids include, but are not limited to, cortisone (hydrocortisone, hydrocortisone sodium phosphate, hydrocortisone sodium succinate, Ala-Cort®, Hydrocort Acetate®, hydrocortone phosphate Lanacort®, Solu-Cortef®), decadron (dexamethasone, dexamethasone acetate, dexamethasone sodium phosphate, Dexasone®, Diodex®, Hexadrol®, Maxidex®), methylprednisolone (6-methylprednisolone, methylprednisolone acetate, methylprednisolone sodium succinate, Duralone®, Medralone®, Medrol®, M-Prednisol®, Solu-Medrol®), prednisolone (Delta-Cortef®, ORAPRED®, Pediapred®, Prezone®), and prednisone (Deltasone®, Liquid Pred®, Meticorten®, Orasone®), and bisphosphonates (e.g., pamidronate (Aredia®), and zoledronic acid (Zometac®).

In some embodiments, the anti-cancer therapy comprises an anti-hormonal agent. In some embodiments, the methods provided herein comprise administering to the individual an anti-hormonal agent, e.g., in combination with another anti-cancer therapy. Anti-hormonal agents are agents that act to regulate or inhibit hormone action on tumors. Examples of anti-hormonal agents include anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGACE® megestrol acetate, AROMASIN® exemestane, formestanie, fadrozole, RIVISOR® vorozole, FEMARA® letrozole, and ARIMIDEX® (anastrozole); anti-androgens such as flutamide, nilutamide, bicalutamide, leuprolide, and goserelin; troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

In some embodiments, the anti-cancer therapy comprises an antimetabolite chemotherapeutic agent. In some embodiments, the methods provided herein comprise administering to the individual an antimetabolite chemotherapeutic agent, e.g., in combination with another anti-cancer therapy. Antimetabolite chemotherapeutic agents are agents that are structurally similar to a metabolite, but cannot be used by the body in a productive manner. Many antimetabolite chemotherapeutic agents interfere with the production of RNA or DNA. Examples of antimetabolite chemotherapeutic agents include gemcitabine (GEMZAR®), 5-fluorouracil (5-FU), capecitabine (XELODA™), 6-mercaptopurine, methotrexate, 6-thioguanine, pemetrexed, raltitrexed, arabinosylcytosine ARA-C cytarabine (CYTOSAR-U®), dacarbazine (DTIC-DOMED), azocytosine, deoxycytosine, pyridmidene, fludarabine (FLUDARA®), cladrabine, and 2-deoxy-D-glucose. In some embodiments, an antimetabolite chemotherapeutic agent is gemcitabine. Gemcitabine HCl is sold by Eli Lilly under the trademark GEMZAR®.

In some embodiments, the anti-cancer therapy comprises a platinum-based chemotherapeutic agent. In some embodiments, the methods provided herein comprise administering to the individual a platinum-based chemotherapeutic agent, e.g., in combination with another anti-cancer therapy. Platinum-based chemotherapeutic agents are chemotherapeutic agents that comprise an organic compound containing platinum as an integral part of the molecule. In some embodiments, a chemotherapeutic agent is a platinum agent. In some such embodiments, the platinum agent is selected from cisplatin, carboplatin, oxaliplatin, nedaplatin, triplatin tetranitrate, phenanthriplatin, picoplatin, or satraplatin.

In some aspects, provided herein are therapeutic formulations comprising an anti-cancer therapy provided herein, and a pharmaceutically acceptable carrier, excipient, or stabilizer. A formulation provided herein may contain more than one active compound, e.g., an anti-cancer therapy provided herein and one or more additional agents (e.g., anti-cancer agents).

Acceptable carriers, excipients, or stabilizers are non-toxic to recipients at the dosages and concentrations employed, and include, for example, one or more of: buffers such as phosphate, citrate, and other organic acids; antioxidants, including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, or m-cresol; low molecular weight polypeptides (e.g., less than about 10 residues); proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); surfactants such as non-ionic surfactants; or polymers such as polyethylene glycol (PEG).

The active ingredients may be entrapped in microcapsules. Such microcapsules may be prepared, for example, by coacervation techniques or by interfacial polymerization, for example, hydroxymethylcellulose or gelatin-microcapsules and poly-(methylmethacylate) microcapsules, respectively; in colloidal drug delivery systems (for example, liposomes, albumin microspheres, microemulsions, nano-particles and nano-capsules); or in macroemulsions. Such techniques are known in the art.

Sustained-release compositions may be prepared. Suitable examples of sustained-release compositions include semi-permeable matrices of solid hydrophobic polymers containing an anti-cancer therapy of the disclosure. Such matrices may be in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

A formulation provided herein may also contain more than one active compound, for example, those with complementary activities that do not adversely affect each other. The type and effective amounts of such medicaments depend, for example, on the amount and type of active compound(s) present in the formulation, and clinical parameters of the subjects.

For general information concerning formulations, see, e.g., Gilman et al. (eds.) The Pharmacological Bases of Therapeutics, 8th Ed., Pergamon Press, 1990; A. Gennaro (ed.), Remington's Pharmaceutical Sciences, 18th Edition, Mack Publishing Co., Pennsylvania, 1990; Avis et al. (eds.) Pharmaceutical Dosage Forms: Parenteral Medications Dekker, New York, 1993; Lieberman et al. (eds.) Pharmaceutical Dosage Forms: Tablets Dekker, New York, 1990; Lieberman et al. (eds.), Pharmaceutical Dosage Forms: Disperse Systems Dekker, New York, 1990; and Walters (ed.) Dermatological and Transdermal Formulations (Drugs and the Pharmaceutical Sciences), Vol 1 19, Marcel Dekker, 2002.

Formulations to be used for in vivo administration are sterile. This is readily accomplished by filtration through sterile filtration membranes or other methods known in the art.

In some embodiments, the anti-cancer therapy is administered as a monotherapy. In some embodiments, the anti-cancer therapy is administered in combination with one or more additional anti-cancer therapies or treatments. In some embodiments, the one or more additional anti-cancer therapies or treatments include one or more anti-cancer therapies described herein. In some embodiments, the methods of the present disclosure comprise administration of any combination of any of the anti-cancer therapies provided herein. In some embodiments, the additional anti-cancer therapy comprises one or more of surgery, radiotherapy, chemotherapy, anti-angiogenic therapy, anti-DNA repair therapy, and anti-inflammatory therapy. In some embodiments, the additional anti-cancer therapy comprises an anti-neoplastic agent, a chemotherapeutic agent, a growth inhibitory agent, an anti-angiogenic agent, a radiation therapy, a cytotoxic agent, or combinations thereof. In some embodiments, an anti-cancer therapy may be administered in conjunction with a chemotherapy or chemotherapeutic agent. In some embodiments, the chemotherapy or chemotherapeutic agent is a platinum-based agent (including, without limitation cisplatin, carboplatin, oxaliplatin, and staraplatin). In some embodiments, an anti-cancer therapy may be administered in conjunction with a radiation therapy. In some embodiments, the anti-cancer therapy for use in any of the methods described herein (e.g., as monotherapy or in combination with another therapy or treatment) is an anti-cancer therapy or treatment described by Pietrantonio et al., J Natl Cancer Inst (2017) 109(12) and/or by Wang et al., Cancers (2020) 12(2):426, which are hereby incorporated by reference.

### Kits

Also provided herein are kits for improving sequencing analysis and/or extracting nucleic acids according to any one of the methods described herein.

In some embodiments, the kit includes reagents and instructions for performing the methods of the present disclosure.

Also provided herein are kits for detecting a biomarker nucleic acid molecule of the disclosure, e.g., in the RNA and/or DNA sample extracted from an embedded sample, as described herein. In some embodiments, a kit provided herein comprises a reagent (e.g., one or more oligonucleotides, primers, probes or baits of the present disclosure) for detecting a biomarker nucleic acid molecule provided herein. In some embodiments, the kit comprises a reagent (e.g., one or more oligonucleotides, primers, probes or baits of the present disclosure) for detecting a wild-type counterpart of a biomarker nucleic acid molecule provided herein. In some embodiments, the reagent comprises one or more oligonucleotides, primers, probes or baits of the present disclosure capable of hybridizing to a biomarker nucleic acid molecule provided herein, or to a wild-type counterpart of a biomarker nucleic acid molecule provided herein. In some embodiments, the reagent comprises one or more oligonucleotides, primers, probes or baits of the present disclosure capable of distinguishing a biomarker nucleic acid molecule provided herein from a wild-type counterpart of the biomarker nucleic acid molecule provided herein. In some embodiments, the kit is for use according to any method of detecting biomarker nucleic acid molecules known in the art or described herein, such as sequencing, PCR, in situ hybridization methods, a nucleic acid hybridization assay, an amplification-based assay, a PCR-RFLP assay, real-time PCR, sequencing, next-generation sequencing, a screening analysis, FISH, spectral karyotyping, MFISH, comparative genomic hybridization, in situ hybridization, sequence-specific priming (SSP) PCR, HPLC, and mass-spectrometric genotyping. In some embodiments, a kit provided herein further comprises instructions for detecting a biomarker nucleic acid molecule of the disclosure, e.g., using one or more oligonucleotides, primers, probes or baits of the present disclosure.

### EXEMPLARY EMBODIMENTS

The following exemplary embodiments are representative of some aspects of the invention:

Embodiment 1. A method of detecting alterations in RNA and/or DNA, wherein the method comprises: a) providing a paraffin-embedded sample; b) removing paraffin from the paraffin-embedded sample, thereby generating a deparaffinized sample, wherein the paraffin is removed by inducing a phase transition in the paraffin and separating the paraffin from the sample; c) extracting RNA and/or DNA from the deparaffinized sample; and d) analyzing the RNA and/or DNA to detect alterations in the RNA and/or DNA.

Embodiment 2. The method of embodiment 1, wherein the detection of alterations in the RNA and/or DNA is improved relative to a method wherein the paraffin is dissolved with a miscible solvent, optionally wherein the miscible solvent is xylene, ethyl acetate, CitriSolv™, or UltraClear™.

Embodiment 3. The method of embodiment 1, wherein the detection of alterations in the RNA and/or DNA is improved relative to a method wherein the paraffin is removed by cutting the paraffin from the paraffin-embedded sample.

Embodiment 4. The method of embodiment 1, wherein the detection of alterations in the RNA and/or DNA is improved relative to a method wherein the paraffin-embedded sample is not deparaffinized.

Embodiment 5. The method of any one of embodiments 1-4, wherein the alteration in the RNA and/or DNA is selected from the group consisting of: a) a copy number alteration; b) a point mutation; c) an in-frame deletion of one or more codons; d) an intragenic deletion; e) an intragenic insertion; f) a deletion of a full gene; g) an inversion; h) an interchromosomal translocation; i) a tandem duplication; j) a gene fusion; k) a genomic rearrangement that comprises an intron sequence; and/or 1) a gene amplification or duplication.

Embodiment 6. The method of any one of embodiments 1-5, wherein the alteration in the RNA and/or the DNA is a copy number alteration.

Embodiment 7. A method of extracting RNA and/or DNA, wherein the method comprises: a) providing a paraffin-embedded sample; b) removing paraffin from the paraffin-embedded sample, thereby generating a deparaffinized sample, wherein the paraffin is removed by inducing a phase transition in the paraffin and separating the paraffin from the sample; and c) extracting RNA and/or DNA from the deparaffinized sample.

Embodiment 8. The method of embodiment 7, wherein the extraction of the RNA and/or DNA is improved relative to a method wherein the paraffin is dissolved with a miscible solvent, optionally wherein the miscible solvent is xylene, ethyl acetate, CitriSolv™ or UltraClear™.

Embodiment 9. The method of embodiment 7, wherein the extraction of the RNA and/or DNA is improved relative to a method wherein the paraffin is removed by cutting the paraffin from the paraffin-embedded sample.

Embodiment 10. A method of improving library construction for nucleic acid sequencing, wherein the method comprises: a) providing a paraffin-embedded sample; b) removing paraffin from the paraffin-embedded sample, thereby generating a deparaffinized sample, wherein the paraffin is removed by inducing a phase transition in the paraffin and separating the paraffin from the sample; c) extracting RNA and/or DNA from the deparaffinized sample; and d) preparing a sequencing library for sequencing the RNA and/or the DNA.

Embodiment 11. The method of embodiment 10, wherein the preparation of the sequencing library is improved relative to a method wherein the paraffin is dissolved with a miscible solvent, optionally wherein the miscible solvent is xylene, ethyl acetate, CitriSolv™ or UltraClear™.

Embodiment 12. The method of embodiment 10, wherein the preparation of the sequencing library is improved relative to a method wherein the paraffin is removed by cutting the paraffin from the paraffin-embedded sample.

Embodiment 13. A method of reducing the level of paraffin in an RNA or DNA sample extracted from a paraffin-embedded sample, wherein the method comprises: a) providing a paraffin-embedded sample; b) removing paraffin from the paraffin-embedded sample, thereby generating a deparaffinized sample, wherein the paraffin is removed by inducing a phase transition in the paraffin and separating the paraffin from the sample; c) extracting RNA and/or DNA from the deparaffinized sample; and d) purifying the extracted RNA and/or DNA to provide an RNA and/or DNA sample.

Embodiment 14. The method of embodiment 13, wherein the level of paraffin in the RNA and/or DNA sample is reduced relative to a method wherein the paraffin is dissolved with a miscible solvent, optionally wherein the miscible solvent is xylene, ethyl acetate, CitriSolv™, or UltraClear™.

Embodiment 15. The method of embodiment 13, wherein the level of paraffin in the RNA and/or DNA sample is reduced relative to a method wherein the paraffin is removed by cutting the paraffin from the paraffin-embedded sample.

Embodiment 16. A method of improving separation of paraffin from a paraffin-embedded sample, wherein the method comprises: a) providing a paraffin-embedded sample; b) removing paraffin from the paraffin-embedded sample, thereby generating a deparaffinized sample, wherein the paraffin is removed by inducing a phase transition in the paraffin and separating the paraffin from the sample; and c) extracting RNA and/or DNA from the deparaffinized sample.

Embodiment 17. The method of embodiment 16, wherein the separation of paraffin from the paraffin-embedded sample is improved relative to a method wherein the paraffin is dissolved with a miscible solvent, optionally wherein the miscible solvent is xylene, ethyl acetate, CitriSolv™, or UltraClear™.

Embodiment 18. The method of embodiment 16, wherein the separation of paraffin from the paraffin-embedded sample is improved relative to a method wherein the paraffin is removed by cutting the paraffin from the paraffin-embedded sample.

Embodiment 19. The method of any one of embodiments 16-18, wherein the method is performed in a liquid handling robot comprising a filter, wherein the improved separation of paraffin results in less clogging of the filter relative to a method wherein the paraffin is dissolved with a miscible solvent, optionally wherein the miscible solvent is xylene, ethyl acetate, CitriSolv™, or UltraClear™.

Embodiment 20. The method of any one of embodiments 16-19, wherein the method is performed in a liquid handling robot comprising a filter, wherein the improved separation of paraffin results in less clogging of the filter relative to a method wherein the paraffin is removed by cutting the paraffin from the paraffin-embedded sample.

Embodiment 21. The method of embodiment 19 or embodiment 20, wherein the filter is a filter in a spin column.

Embodiment 22. The method of any one of the preceding embodiments, wherein step b) does not comprise dissolving the paraffin with a miscible solvent, optionally wherein the miscible solvent is xylene, ethyl acetate, CitriSolv™, or UltraClear™.

Embodiment 23. The method of any one of the preceding embodiments, wherein the phase transition is melting.

Embodiment 24. The method of any one of the preceding embodiments, wherein step b) comprises heating and centrifuging the paraffin-embedded sample.

Embodiment 25. The method of embodiment 24, wherein the paraffin-embedded sample is heated to about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., or about 75° C.

Embodiment 26. The method of embodiment 24 or embodiment 25, wherein the paraffin-embedded sample is heated for about 5 minutes, about 10 minutes, about 15 minutes, about 20 minutes, about 25 minutes, about 30 minutes, or about 35 minutes.

Embodiment 27. The method of any one of the preceding embodiments, wherein step b) comprises centrifuging and filtering the paraffin-embedded sample.

Embodiment 28. The method of embodiment 27, wherein the paraffin-embedded sample is centrifuged at about 1,700 rcf, about 1,750 rcf, about 1,800 rcf, about 1,850 rcf, or about 1,900 rcf.

Embodiment 29. The method of embodiment 27 or embodiment 28, wherein the paraffin-embedded sample is centrifuged at 1,811 rcf or greater.

Embodiment 30. The method of any one of embodiments 27-29, wherein the paraffin-embedded sample is centrifuged at 1,811 rcf.

Embodiment 31. The method of any one of embodiments 1-28, wherein the paraffin-embedded sample is centrifuged at 1600 to 2000 rcf.

Embodiment 32. The method of embodiment 31, wherein the paraffin-embedded sample is heated to about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., or about 75° C.

Embodiment 33. The method of embodiment 31 or embodiment 32, wherein the paraffin-embedded sample is heated for about 5 minutes, about 10 minutes, about 15 minutes, about 20 minutes, about 25 minutes, about 30 minutes, or about 35 minutes.

Embodiment 34. The method of any one embodiments 31-33, wherein the paraffin-embedded sample is centrifuged at about 1,700 rcf, about 1,750 rcf, about 1,800 rcf, about 1,850 rcf, or about 1,900 ref.

Embodiment 35. The method of any one embodiments 31-34, wherein the paraffin-embedded sample is centrifuged at 1,811 ref or greater.

Embodiment 36. The method of any one embodiments 31-35, wherein the paraffin-embedded sample is centrifuged at 1,811 ref.

Embodiment 37. The method of embodiment 24 or 27-31, wherein the paraffin-embedded sample is heated to about 50° C. to about 80° C.

Embodiment 38. A method of detecting alterations in RNA and/or DNA, wherein the method comprises: a) providing a paraffin-embedded sample; b) removing paraffin from the paraffin-embedded sample, thereby generating a deparaffinized sample, wherein the paraffin is removed by contacting the paraffin-embedded sample with an immiscible solvent and separating the paraffin from the sample; c) extracting RNA and/or DNA from the deparaffinized sample; and d) analyzing the RNA and/or DNA to detect alterations in the RNA and/or DNA.

Embodiment 39. The method of embodiment 38, wherein the detection of alterations in the RNA and/or DNA is improved relative to a method wherein the paraffin is dissolved with a miscible solvent, optionally wherein the miscible solvent is xylene, ethyl acetate, CitriSolv™, or UltraClear™.

Embodiment 40. The method of embodiment 38, wherein the detection of alterations in the RNA and/or DNA is improved relative to a method wherein the paraffin is removed by cutting the paraffin from the paraffin-embedded sample.

Embodiment 41. The method of embodiment 38, wherein the detection of alterations in the RNA and/or DNA is improved relative to a method wherein the paraffin-embedded sample is not deparaffinized.

Embodiment 42. The method of any one of embodiments 38-41, wherein the alteration in the RNA and/or DNA is selected from the group consisting of: a) a copy number alteration; b) a point mutation; c) an in-frame deletion of one or more codons; d) an intragenic deletion; e) an intragenic insertion; f) a deletion of a full gene; g) an inversion; h) an interchromosomal translocation; i) a tandem duplication; j) a gene fusion; k) a genomic rearrangement that comprises an intron sequence; and/or 1) a gene amplification or duplication.

Embodiment 43. The method of any one of embodiments 38-42, wherein the alteration in the RNA and/or the DNA is a copy number alteration.

Embodiment 44. A method of extracting RNA and/or DNA, wherein the method comprises: a) providing a paraffin-embedded sample; b) removing paraffin from the paraffin-embedded sample, thereby generating a deparaffinized sample, wherein the paraffin is removed by contacting the paraffin-embedded sample with an immiscible solvent and separating the paraffin from the sample; and c) extracting RNA and/or DNA from the deparaffinized sample.

Embodiment 45. The method of embodiment 44, wherein the extraction of the RNA and/or DNA is improved relative to a method wherein the paraffin is dissolved with a miscible solvent, optionally wherein the miscible solvent is xylene, ethyl acetate, CitriSolv™ or UltraClear™.

Embodiment 46. The method of embodiment 44, wherein the extraction of the RNA and/or DNA is improved relative to a method wherein the paraffin is removed by cutting the paraffin from the paraffin-embedded sample.

Embodiment 47. A method of improving library construction for nucleic acid sequencing, wherein the method comprises: a) providing a paraffin-embedded sample; b) removing paraffin from the paraffin-embedded sample, thereby generating a deparaffinized sample, wherein the paraffin is removed by contacting the paraffin-embedded sample with an immiscible solvent and separating the paraffin from the sample; c) extracting RNA and/or DNA from the deparaffinized sample; and d) preparing a sequencing library for sequencing the RNA and/or the DNA.

Embodiment 48. The method of embodiment 47, wherein the preparation of the sequencing library is improved relative to a method wherein the paraffin is dissolved with a miscible solvent, optionally wherein the miscible solvent is xylene, ethyl acetate, CitriSolv™ or UltraClear™.

Embodiment 49. The method of embodiment 47, wherein the preparation of the sequencing library is improved relative to a method wherein the paraffin is removed by cutting the paraffin from the paraffin-embedded sample.

Embodiment 50. A method of reducing the level of paraffin in an RNA or DNA sample extracted from a paraffin-embedded sample, wherein the method comprises: a) providing a paraffin-embedded sample; b) removing paraffin from the paraffin-embedded sample, thereby generating a deparaffinized sample, wherein the paraffin is removed by contacting the paraffin-embedded sample with an immiscible solvent and separating the paraffin from the sample; c) extracting RNA and/or DNA from the deparaffinized sample; and d) purifying the extracted RNA and/or DNA to provide an RNA and/or DNA sample.

Embodiment 51. The method of embodiment 50, wherein the level of paraffin in the RNA and/or DNA sample is reduced relative to a method wherein the paraffin is dissolved with a miscible solvent, optionally wherein the miscible solvent is xylene, ethyl acetate, CitriSolv™, or UltraClear™.

Embodiment 52. The method of embodiment 50, wherein the level of paraffin in the RNA and/or DNA sample is reduced relative to a method wherein the paraffin is removed by cutting the paraffin from the paraffin-embedded sample.

Embodiment 53. A method of improving separation of paraffin from a paraffin-embedded sample, wherein the method comprises: a) providing a paraffin-embedded sample; b) removing paraffin from the paraffin-embedded sample, thereby generating a deparaffinized sample, wherein the paraffin is removed by contacting the paraffin-embedded sample with an immiscible solvent and separating the paraffin from the sample; and c) extracting RNA and/or DNA from the deparaffinized sample.

Embodiment 54. The method of embodiment 53, wherein the separation of paraffin from the paraffin-embedded sample is improved relative to a method wherein the paraffin is dissolved with a miscible solvent, optionally wherein the miscible solvent is xylene, ethyl acetate, CitriSolv™, or UltraClear™.

Embodiment 55. The method of embodiment 53, wherein the separation of paraffin from the paraffin-embedded sample is improved relative to a method wherein the paraffin is removed by cutting the paraffin from the paraffin-embedded sample.

Embodiment 56. The method of any one of embodiments 53-55, wherein the method is performed in a liquid handling robot comprising a filter, wherein the improved separation of paraffin results in less clogging of the filter relative to a method wherein the paraffin is dissolved with a miscible solvent, optionally wherein the miscible solvent is xylene, ethyl acetate, CitriSolv™, or UltraClear™.

Embodiment 57. The method of any one of embodiments 53-56, wherein the method is performed in a liquid handling robot comprising a filter, wherein the improved separation of paraffin results in less clogging of the filter relative to a method wherein the paraffin is removed by cutting the paraffin from the paraffin-embedded sample.

Embodiment 58. The method of embodiment 56 or embodiment 57, wherein the filter is a filter in a spin column.

Embodiment 59. The method of any one of embodiments 38-57, wherein step b) does not comprise dissolving the paraffin with a miscible solvent, optionally wherein the miscible solvent is xylene, ethyl acetate, CitriSolv™, or UltraClear™.

Embodiment 60. The method of any one of embodiments 38-59, wherein the immiscible solvent is mineral oil.

Embodiment 61. The method of embodiment 60, wherein the paraffin-embedded sample is contacted with about 300, 350, 400, 450, 500, or 550 μL of mineral oil.

Embodiment 62. The method of embodiment 60 or embodiment 61, wherein the paraffin-embedded sample is contacted with mineral oil at a mineral oil to paraffin-embedded sample ratio of about 1:4, 1:2, 1:1, 2:1, 4:1, 5:1, 6:1, 7:1, 8:1, 9:1, 10:1, 15:1, 20:1, 30:1, 40:1, or 50:1.

Embodiment 63. The method of any one of embodiments 60-62, wherein step b) comprises incubating the paraffin-embedded sample in mineral oil at about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., or about 75° C.

Embodiment 64. The method of any one of embodiments 60-63, wherein the mineral oil contacts the paraffin-embedded sample for about 5 minutes, about 10 minutes, about 15 minutes, about 20 minutes, about 25 minutes, about 30 minutes, or about 35 minutes.

Embodiment 65. The method of any one of embodiments 60-64, wherein the mineral oil contacts the paraffin-embedded sample while shaking at about 25 rpm, about 50 rpm, about 100 rpm, about 200 rpm, about 300 rpm, about 400 rpm, about 500 rpm, about 600 rpm, about 700 rpm, about 800 rpm, about 900 rpm, about 1000 rpm, or about 1100 rpm.

Embodiment 66. The method of any one of embodiments 38-65, wherein step b) comprises contacting the sample with an immiscible solvent, centrifuging, and filtering the paraffin-embedded sample.

Embodiment 67. The method of embodiment 66, wherein the paraffin-embedded sample is centrifuged at about 1,700 rcf, about 1,750 rcf, about 1,800 rcf, about 1,850 rcf, or about 1,900 ref.

Embodiment 68. The method of embodiment 66 or embodiment 67, wherein the paraffin-embedded sample is centrifuged at 1,811 ref or greater.

Embodiment 69. The method of any one of embodiments 66-68, wherein the paraffin-embedded sample is centrifuged at 1,811 ref.

Embodiment 70. The method of any one of the preceding embodiments, wherein the separation of the paraffin from the sample is automated.

Embodiment 71. The method of any one of the preceding embodiments, wherein step b) is automated.

Embodiment 72. The method of any one of the preceding embodiments, wherein the method is automated.

Embodiment 73. The method of any one of the preceding embodiments, wherein two or more paraffin-embedded samples are processed in parallel.

Embodiment 74. The method of embodiment 73, wherein 12, 24, 48, or 96 paraffin-embedded samples are processed in parallel.

Embodiment 75. The method of any one of the preceding embodiments, wherein the method is performed using a liquid handling robot.

Embodiment 76. The method of embodiment 75, wherein the liquid handling robot is an Agilent, BioTek, Hamilton, Tecan, or ThermoFisher Scientific liquid handling robot, optionally wherein the liquid handling robot is a Hamilton AutoLys STAR, a Hamilton STAR, a BioTek Dispenser, or a KingFisher Flex.

Embodiment 77. The method of embodiment 75 or 76, wherein the liquid handling robot is a Hamilton AutoLys STAR.

Embodiment 78. The method of any one of the preceding embodiments, wherein step c) comprises extracting RNA and DNA from the deparaffinized sample.

Embodiment 79. The method of any one of the preceding embodiments, wherein step a) comprises: i) identifying a target region comprising tumor cells of interest in a paraffin-embedded tissue; ii) extracting the paraffin-embedded sample from the paraffin-embedded tissue; iii) identifying the location of the paraffin-embedded sample in the paraffin-embedded tissue; and iv) if the location of the paraffin-embedded sample overlaps with the target region comprising tumor cells of interest, extracting RNA and/or DNA from the sample or proceeding with step b.

Embodiment 80. The method of embodiment 79, wherein if the location of the paraffin-embedded sample does not overlap with the target region comprising tumor cells of interest, steps ii) and iii) are repeated.

Embodiment 81. The method of embodiment 79 or embodiment 80, wherein step ii) comprises extracting the paraffin-embedded sample using a needle.

Embodiment 82. The method of embodiment 81, wherein the needle is punched through the paraffin-embedded tissue, thereby extracting the paraffin-embedded sample.

Embodiment 83. The method of embodiment 81 or embodiment 82, wherein the needle is a disposable needle.

Embodiment 84. The method of any one of embodiments 81-83, wherein the needle is a 13 gauge needle, 14 gauge needle, a 15 gauge needle, a 16 gauge needle, a 17 gauge needle, a 18 gauge needle, a 19 gauge needle, a 20 gauge needle, or a 21 gauge needle.

Embodiment 85. The method of any one of embodiments 79-84, wherein the paraffin-embedded sample extracted from the paraffin-embedded tissue is about 0.8, 0.9, 1.0, 1.2, 1.4, 1.6, 1.8, 2.0, or 2.3 mm in diameter.

Embodiment 86. The method of embodiment 79 or embodiment 80, wherein step ii) comprises extracting the paraffin-embedded sample using laser microdissection (LMD) or a razor blade.

Embodiment 87. The method of any one of embodiments 79-86, wherein step iii) comprises preparing a slide of a section of the paraffin-embedded tissue.

Embodiment 88. The method of embodiment 87, wherein the section of the paraffin-embedded tissue is stained.

Embodiment 89. The method of embodiment 87 or embodiment 88, wherein the section of the paraffin-embedded tissue is Haematoxylin and Eosin (H&E) stained.

Embodiment 90. The method of any one of embodiments 79-89, wherein step iii) is performed by visual inspection.

Embodiment 91. The method of any one of embodiments 79-89, wherein step iii) is performed by a computer system.

Embodiment 92. The method of any one of embodiments 79-89, wherein step iii) is performed using an image analysis system.

Embodiment 93. The method of any one of the preceding embodiments, wherein the paraffin-embedded sample is from an individual known to have cancer or suspected of having cancer.

Embodiment 94. The method of any one of the preceding embodiments, wherein the paraffin-embedded sample is from a biopsy, optionally a tumor biopsy.

Embodiment 95. The method of any one of the preceding embodiments, wherein the paraffin-embedded sample is a fixed paraffin-embedded sample.

Embodiment 96. The method of embodiment 95, wherein the fixed paraffin-embedded sample is selected from the group consisting of a formalin-fixed sample, an ethanol-fixed sample, and a methanol-fixed sample.

Embodiment 97. The method of any one of the preceding embodiments, wherein the paraffin-embedded sample is derived from a formalin-fixed paraffin-embedded (FFPE) tissue.

Embodiment 98. The method of any one of the preceding embodiments, wherein the paraffin-embedded sample is derived from a cryopreserved tissue.

Embodiment 99. The method of any one of the preceding embodiments, wherein the paraffin-embedded sample is derived from a fresh-frozen tissue.

Embodiment 100. The method of embodiment 99, wherein the fresh-frozen tissue is frozen in an optimal cutting temperature (OCT) compound.

Embodiment 101. The method of any one of the preceding embodiments, wherein the RNA is extracted before the DNA is extracted.

Embodiment 102. The method of embodiment 101, wherein the method further comprises digesting the paraffin-embedded sample before step b).

Embodiment 103. The method of embodiment 102, wherein the paraffin-embedded sample is digested using a proteinase.

Embodiment 104. The method of embodiment 103, wherein the proteinase is proteinase K.

Embodiment 105. The method of embodiment 104, wherein the paraffin-embedded sample is digested using about 10 μL, about 15 μL, about 20 μL, about 25 μL, about 30 μL, or about 35 μL of 20 mg/mL proteinase K.

Embodiment 106. The method of any one of embodiments 103-105, wherein the paraffin-embedded sample is incubated with the proteinase at about 50° C., about 51° C., about 52° C., about 53° C., about 54° C., about 55° C., about 56° C., about 57° C., about 58° C., about 59° C., about 60° C., about 61° C., or about 62° C.

Embodiment 107. The method of any one of embodiments 103-106, wherein the paraffin-embedded sample is incubated with the proteinase for about 10 minutes, about 11 minutes, about 12 minutes, about 13 minutes, about 14 minutes, about 15 minutes, about 16 minutes, about 17 minutes, about 18 minutes, about 19 minutes, or about 20 minutes.

Embodiment 108. The method of any one of embodiments 103-107, wherein the paraffin-embedded sample is incubated with the proteinase shaking at about 500 rpm, 600 rpm, 700 rpm, 800 rpm, 900 rpm, 1000 rpm, 1100 rpm, 1200 rpm, 1300 rpm, 1400 rpm, or 1500 rpm.

Embodiment 109. The method of any one of embodiments 102-108, wherein the paraffin-embedded sample is partially digested or completely digested.

Embodiment 110. The method of any one of embodiments 102-109, wherein the method further comprises de-crosslinking the digested sample after step b).

Embodiment 111. The method of embodiment 110, wherein de-crosslinking comprises heating the digested sample to 80-90° C.

Embodiment 112. The method of embodiment 111, wherein the digested sample is heated for about 10 minutes, about 11 minutes, about 12 minutes, about 13 minutes, about 14 minutes, about 15 minutes, about 16 minutes, about 17 minutes, about 18 minutes, about 19 minutes, or about 20 minutes.

Embodiment 113. The method of any one of embodiments 102-112, wherein step c) comprises collecting a sample lysate comprising RNA from the digested paraffin-embedded sample, and purifying the RNA from the sample lysate comprising RNA.

Embodiment 114. The method of embodiment 113, wherein the method further comprises completely digesting the digested paraffin-embedded sample.

Embodiment 115. The method of embodiment 114, wherein the complete digestion is performed using a proteinase.

Embodiment 116. The method of embodiment 115, wherein the proteinase is proteinase K.

Embodiment 117. The method of embodiment 116, wherein the digested paraffin-embedded sample is digested using about 10 μL, about 15 μL, about 20 μL, about 25 μL, about 30 μL, or about 35 μL of 20 mg/mL proteinase K.

Embodiment 118. The method of any one of embodiments 114-117, wherein the digested paraffin-embedded sample is incubated with the proteinase at about 65° C., about 66° C., about 67° C., about 68° C., about 69° C., about 70° C., about 71° C., about 72° C., about 73° C., about 74° C., or about 75° C.

Embodiment 119. The method of any one of embodiments 114-118, wherein the digested paraffin-embedded sample is incubated with the proteinase for about 6 hours, about 7 hours, about 8 hours, about 9 hours, about 10 hours, about 11 hours, 12 hours, about 13 hours, about 14 hours, about 15 hours, about 16 hours, about 17 hours, about 18 hours, about 19 hours, about 20 hours, about 21 hours, or about 22 hours.

Embodiment 120. The method of any one of embodiments 114-119, wherein the digested paraffin-embedded sample is incubated with the proteinase shaking at about 500 rpm, 600 rpm, 700 rpm, 800 rpm, 900 rpm, 1000 rpm, 1100 rpm, 1200 rpm, 1300 rpm, 1400 rpm, or 1500 rpm.

Embodiment 121. The method of any one of embodiments 114-120, wherein the method further comprises collecting a sample lysate comprising DNA from the completely digested paraffin-embedded sample.

Embodiment 122. The method of any one of embodiments 114-121, wherein the method further comprises purifying the DNA from the sample lysate comprising DNA.

Embodiment 123. The method of any one of embodiments 1-100, wherein the DNA is extracted before the RNA is extracted.

Embodiment 124. The method of embodiment 123, wherein the method further comprises completely digesting the paraffin-embedded sample after step b).

Embodiment 125. The method of embodiment 124, wherein the complete digestion is performed using a proteinase.

Embodiment 126. The method of embodiment 125, wherein the proteinase is proteinase K.

Embodiment 127. The method of embodiment 126, wherein the paraffin-embedded sample is digested using about 10 μL, about 15 μL, about 20 μL, about 25 μL, about 30 μL, or about 35 μL of 20 mg/mL proteinase K.

Embodiment 128. The method of any one of embodiments 125-127, wherein the paraffin-embedded sample is incubated with the proteinase at about 65° C., about 66° C., about 67° C., about 68° C., about 69° C., about 70° C., about 71° C., about 72° C., about 73° C., about 74° C., or about 75° C.

Embodiment 129. The method of any one of embodiments 125-128, wherein the paraffin-embedded sample is incubated with the proteinase for about 6 hours, about 7 hours, about 8 hours, about 9 hours, about 10 hours, about 11 hours, 12 hours, about 13 hours, about 14 hours, about 15 hours, about 16 hours, about 17 hours, about 18 hours, about 19 hours, about 20 hours, about 21 hours, or about 22 hours.

Embodiment 130. The method of any one of embodiments 124-129, wherein the method further comprises extracting the DNA from the completely digested paraffin-embedded sample.

Embodiment 131. The method of embodiment 130, wherein the method further comprises extracting the RNA from the completely digested paraffin-embedded sample.

Embodiment 132. The method of any one of the preceding embodiments, wherein the method further comprises measuring the amount of RNA and/or DNA extracted from the paraffin-embedded sample.

Embodiment 133. The method of embodiment 132, wherein the amount of RNA and/or DNA extracted from the paraffin-embedded sample is about 5 ng, about 10 ng, about 20 ng, about 30 ng, about 40 ng, about 50 ng, about 60 ng, about 70 ng, about 80 ng, about 100 ng, about 50 g, or about 50 mg.

Embodiment 134. The method of any one of the preceding embodiments, wherein the method further comprises analyzing the RNA and/or the DNA extracted from the paraffin-embedded sample by one or more methods selected from the group consisting of a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), and mass-spectrometric genotyping.

Embodiment 135. The method of any one of the preceding embodiments, wherein the method further comprises analyzing the RNA and/or the DNA extracted from the paraffin-embedded sample by next-generation sequencing.

Embodiment 136. The method of any one of the preceding embodiments, wherein the method further comprises: e) optionally, ligating one or more adaptors onto the RNA and/or DNA extracted from the paraffin-embedded sample, thereby generating ligated nucleic acids; d) optionally, amplifying nucleic acids from the ligated nucleic acids; f) optionally, capturing a plurality of nucleic acids corresponding to one or more genes of interest; g) sequencing, by a sequencer, the plurality of nucleic acids to obtain a plurality of sequence reads corresponding to the one or more genes of interest; h) analyzing the plurality of sequence reads; and i) based on the analysis, detecting one or more mutations in a gene of interest.

Embodiment 137. The method of embodiment 136, wherein the plurality of nucleic acids corresponding to the one or more genes of interest is captured from the amplified nucleic acids by hybridization with a bait molecule.

Embodiment 138. The method of embodiment 136, wherein the plurality of nucleic acids corresponding to the one or more genes of interest is enriched from the amplified nucleic acids, optionally wherein the plurality of nucleic acids corresponding to the one or more genes of interest is enriched from the amplified nucleic acids by biotin/streptavidin tagging.

Embodiment 139. The method of any one of embodiments 136-138, wherein prior to step e) the RNA and/or DNA extracted from the paraffin-embedded sample are fragmented, optionally wherein the RNA and/or DNA extracted from the paraffin-embedded sample are fragmented by sonication.

Embodiment 140. The method of embodiment 139, wherein the fragmented RNA and/or DNA extracted from the paraffin-embedded sample are end-repaired.

Embodiment 141. The method of embodiment 140, wherein the end-repaired, fragmented RNA and/or DNA extracted from the sample are dA-tailed or dT-tailed.

Embodiment 142. The method of any one of the preceding embodiments, wherein the method further comprises analyzing the DNA extracted from the paraffin-embedded sample to detect a somatic mutation selected from the group consisting of: i) a point mutation; ii) an in-frame deletion of one or more codons; iii) an intragenic deletion; iv) an intragenic insertion; v) a deletion of a full gene; vi) an inversion; vii) an interchromosomal translocation; viii) a tandem duplication; ix) a gene fusion; x) a genomic rearrangement that comprises an intron sequence; and/or xi) a gene amplification or duplication.

Embodiment 143. The method of embodiment 142, wherein the somatic mutation is in a gene, and wherein the gene is ABL1, AKT1, AKT2, AKT3, ALK, APC, AR, BRAF, CCND1, CDK4, CDKN2A, CEBPA, CTNNB1, EGFR, ERBB2, ESR1, FGFR1, FGFR2, FGFR3, FLT3, HRAS, JAK2, KIT, KRAS, MAP2K1, MAP2K2, MET, MLL, MYC, NF1, NOTCH1, NPM1, NRAS, NTRK3, PDGFRA, PIK3CA, PIK3CG, PIK3R1, PTCH1, PTCH2, PTEN, RB1, RET, SMO, STK11, SUFU, or TP53.

Embodiment 144. The method of embodiment 142, wherein the somatic mutation is in a gene, and wherein the gene is ABL2, ARAF, ARFRP1, ARID1A, ATM, ATR, AURKA, AURKB, BAP1, BCL2, BCL2A1, BCL2L1, BCL2L2, BCL6, BRCA1, BRCA2, CBL, CARD11, CBL, CCND2, CCND3, CCNE1, CD79A, CD79B, CDH1, CDH2, CDH20, CDH5, CDK6, CDK8, CDKN2B, CDKN2C, CHEK1, CHEK2, CRKL, CRLF2, DNMT3A, DOT1L, EPHA3, EPHA5, EPHA6, EPHA7, EPHB1, EPHB4, EPHB6, ERBB3, ERBB4, ERG, ETV1, ETV4, ETV5, ETV6, EWSR1, EZH2, FANCA, FBXW7, FGFR4, FLT1, FLT4, FOXP4, GATA1, GNA11, GNAQ, GNAS, GPR124, GUCY1A2, HOXA3, HSP90AA1, IDH1, IDH2, IGF1R, IGF2R, IKBKE, IKZF1, INHBA, IRS2, JAK1, JAK3, JUN, KDM6A, KDR, LRP1B, LRP6, LTK, MAP2K4, MCL1, MDM2, MDM4, MEN1, MITF, MLH1, MPL, MRE11A, MSH2, MSH6, MTOR, MUTYH, MYCL1, MYCN, NF2, NKX2-1, NTRK1, NTRK2, PAK3, PAX5, PDGFRB, PKHD1, PLCG1, PRKDC, PTPN11, PTPRD, RAF1, RARA, RICTOR, RPTOR, RUNX1, SMAD2, SMAD3, SMAD4, SMARCA4, SMARCB1, SOX10, SOX2, SRC, TBX22, TET2, TGFBR2, TMPRSS2, TNFAIP3, TNK, TNKS2, TOP1, TSC1, TSC2, USP9X, VHL, or WT1.

Embodiment 145. The method of any one of the preceding embodiments, wherein the method further comprises analyzing the RNA extracted from the paraffin-embedded sample to detect an alternation selected from the group consisting of: i) gene fusions; ii) exon-skipping events; iii) splice variants; and/or iv) altered gene expression.

Embodiment 146. The method of any one of the preceding embodiments, wherein the method further comprises detecting loss-of-heterozygosity (LOH) of one or more genes of interest in the paraffin-embedded sample.

Embodiment 147. The method of embodiment 146, wherein the method further comprises detecting LOH of ST7/RAY1, ARH1/NOEY2, TSLC1, RB, PTEN, SMAD2, SMAD4, DCC, TP53, ATM, miR-15a, miR-16-1, NAT2, BRCA1, BRCA2, hOGG1, CDH1, IGF2, CDKN1C/P57, MEN1, PRKAR1A, H19, KRAS, BAP1, PTCH1, SMO, SUFU, NOTCH1, PPP6C, LATS1, CASP8, PTPN14, ARID1A, FBXW7, M6P/IGF2R, IFN-alpha, an olfactory receptor gene, CBFA2T3, DUTT1, FHIT, APC, P16, FCMD, TSC2, miR-34, c-MPL, RUNX3, DIRAS3, NRAS, miR-9, FAM50B, PLAGL1, ER, FLT3, ZDBF2, GPR1, c-KIT, NAP1L5, GRB10, EGFR, PEG10, BRAF, MEST, JAK2, DAPK1, LIT1, WT1, NF-1, PR, c-CBL, DLK1, AKT1, SNURF, a cytochrome P450 gene (CYP), ZNF587, SOCS1, TIMP2, RUNX1, AR, CEBPA, C19MC, EMP3, ZNF331, CDKN2A, PEG3, NNAT, GNAS, and/or GATA5 in the paraffin-embedded sample.

Embodiment 148. The method of embodiment 146, wherein the method further comprises detecting LOH of a human leukocyte antigen (HLA) gene in the paraffin-embedded sample.

Embodiment 149. The method of embodiment 148, wherein the method further comprises: ligating one or more adaptors onto one or more of the RNA and/or DNA extracted from the paraffin-embedded sample, thereby generating ligated nucleic acids; amplifying the ligated nucleic acids; capturing a plurality of nucleic acids corresponding to the HLA gene from the amplified nucleic acids using a bait molecule; sequencing the captured nucleic acids to obtain a plurality of sequence reads corresponding to the HLA gene; fitting, by one or more processors, one or more values associated with one or more of the plurality of sequence reads to a model; and based on the model, detecting LOH of the HLA gene and a relative binding propensity for an HLA allele of the HLA gene.

Embodiment 150. The method of embodiment 149, wherein LOH of the HLA gene and relative binding propensity for an HLA allele of the HLA gene are detected by: a) obtaining an observed allele frequency for an HLA allele, wherein observed allele frequency corresponds to frequency of nucleic acid(s) encoding at least a portion of the HLA allele as detected among the plurality of sequence reads corresponding to the HLA gene; b) obtaining a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles; c) applying an objective function to measure a difference between the relative binding propensity and the observed allele frequency of the HLA allele; d) applying an optimization model to minimize the objective function; e) determining an adjusted allele frequency of the HLA allele based on the optimization model and the observed allele frequency; and f) determining that LOH has occurred when the adjusted allele frequency of the HLA allele is less than a predetermined threshold.

Embodiment 151. The method of embodiment 149 or embodiment 150, wherein the paraffin-embedded sample is from an individual known to have cancer or suspected of having cancer, further comprising, based at least in part on detection of LOH of the HLA gene, administering an effective amount of a treatment other than an immune checkpoint inhibitor (ICI) to the individual.

Embodiment 152. The method of embodiment 149 or embodiment 150, further comprising, based at least in part on detection of LOH of the HLA gene, recommending a treatment other than an immune checkpoint inhibitor (ICI).

Embodiment 153. The method of embodiment 149 or embodiment 150, further comprising: detecting, or acquiring knowledge of, a high tumor mutational burden (TMB) in the paraffin-embedded sample.

Embodiment 154. The method of embodiment 153, wherein the paraffin-embedded sample is from an individual known to have cancer or suspected of having cancer, further comprising, based at least in part on detection of LOH of the HLA gene and high TMB, administering an effective amount of an immune checkpoint inhibitor (ICI) to the individual.

Embodiment 155. The method of embodiment 153, wherein the paraffin-embedded sample is from an individual known to have cancer or suspected of having cancer, further comprising, based at least in part on detection of LOH of the HLA gene and high TMB, recommending a treatment comprising an immune checkpoint inhibitor (ICI) to the individual.

Embodiment 156. The method of any one of embodiments 148-155, wherein the HLA gene is a human HLA-A, HLA-B, or HLA-C gene.

Embodiment 157. The method of any one of embodiments 153-156, wherein the TMB is determined based on a number of non-driver somatic coding mutations per megabase of genome sequenced.

Embodiment 158. The method of any one of embodiments 1-144, wherein the method further comprises detecting a loss-of-function mutation in a phosphatase and tensin homolog (PTEN) gene in the paraffin-embedded sample.

Embodiment 159. The method of embodiment 158, wherein the loss-of-function mutation in a PTEN gene comprises one or more of an insertion, deletion or substitution of one or more nucleotides, a genomic rearrangement, an alteration in a promoter, a gene fusion, or a copy number alteration.

Embodiment 160. The method of embodiment 158 or embodiment 159, wherein the loss-of-function mutation in a PTEN gene is detected in the paraffin-embedded sample by one or more of: a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping.

Embodiment 161. The method of any one of embodiments 1-144, wherein the method further comprises measuring the level of tumor mutational burden (TMB) in the paraffin-embedded sample.

Embodiment 162. The method of embodiment 161, wherein a TMB of at least about 10 mutations/megabase (Mb) or at least about 20 mut/Mb is detected.

Embodiment 163. The method of embodiment 161 or embodiment 162, wherein TMB is measured in the RNA and/or DNA from the paraffin-embedded sample by whole exome sequencing, whole genome sequencing, or gene-targeted sequencing.

Embodiment 164. The method of embodiment 163, wherein TMB is measured on about 0.80 Mb of sequenced DNA.

Embodiment 165. The method of embodiment 163, wherein TMB is measured on between about 0.83 Mb and about 1.14 Mb of sequenced DNA.

Embodiment 166. The method of embodiment 163, wherein TMB is measured on about 1.1 Mb of sequenced DNA.

Embodiment 167. The method of embodiment 163, wherein TMB is measured on up to about 1.1 Mb of sequenced DNA.

Embodiment 168. The method of any one of embodiments 1-144, wherein the method further comprises detecting homozygous single exon loss in the paraffin-embedded sample.

Embodiment 169. The method of embodiment 168, wherein the homozygous single exon loss is detected in the RNA and/or DNA from the paraffin-embedded sample by whole exome sequencing, whole genome sequencing, or gene-targeted sequencing.

Embodiment 170. A method of detecting an analyte, wherein the method comprises: a) providing an embedded sample comprising an analyte, wherein the sample is embedded in an embedding agent; b) removing the embedding agent from the embedded sample, thereby generating a de-embedded sample, wherein the embedding agent is removed by inducing a phase transition in the embedding agent and separating the embedding agent from the sample; c) extracting the analyte from the de-embedded sample; and d) analyzing the analyte to detect the analyte.

Embodiment 171. The method of embodiment 170, wherein the detection the analyte is improved relative to a method wherein the embedding agent is dissolved with a miscible solvent, optionally wherein the miscible solvent is xylene, ethyl acetate, CitriSolv™, or UltraClear™.

Embodiment 172. The method of embodiment 170, wherein the detection the analyte is improved relative to a method wherein the embedding agent is removed by cutting the embedding agent from the embedded sample.

Embodiment 173. The method of embodiment 170, wherein the detection the analyte is improved relative to a method wherein the embedded sample is not de-embedded.

Embodiment 174. A method of extracting an analyte, wherein the method comprises: a) providing an embedded sample comprising an analyte, wherein the sample is embedded in an embedding agent; b) removing the embedding agent from the embedded sample, thereby generating a de-embedded sample, wherein the embedding agent is removed by inducing a phase transition in the embedding agent and separating the embedding agent from the sample; and c) extracting the analyte from the de-embedded sample.

Embodiment 175. The method of embodiment 174, wherein the extraction of the analyte is improved relative to a method wherein the embedding agent is dissolved with a miscible solvent, optionally wherein the miscible solvent is xylene, ethyl acetate, CitriSolv™ or UltraClear™.

Embodiment 176. The method of embodiment 174, wherein the extraction of the analyte is improved relative to a method wherein the embedding agent is removed by cutting the embedding agent from the embedded sample.

Embodiment 177. A method of reducing the level of embedding agent in an analyte sample extracted from an embedded sample, wherein the method comprises: a) providing an embedded sample comprising an analyte, wherein the sample is embedded in an embedding agent; b) removing the embedding agent from the embedded sample, thereby generating a de-embedded sample, wherein the embedding agent is removed by inducing a phase transition in the embedding agent and separating the embedding agent from the sample; c) extracting the analyte from the de-embedded sample; and d) purifying the extracted analyte to provide an analyte sample.

Embodiment 178. The method of embodiment 177, wherein the level of embedding agent in the analyte sample is reduced relative to a method wherein the embedding agent is dissolved with a miscible solvent, optionally wherein the miscible solvent is xylene, ethyl acetate, CitriSolv™, or UltraClear™.

Embodiment 179. The method of embodiment 177, wherein the level of embedding agent in the analyte sample is reduced relative to a method wherein the embedding agent is removed by cutting the embedding agent from the embedded sample.

Embodiment 180. A method of improving separation of an embedding agent from an embedded sample, wherein the method comprises: a) providing an embedded sample comprising an analyte, wherein the sample is embedded in an embedding agent; b) removing the embedding agent from the embedded sample, thereby generating a de-embedded sample, wherein the embedding agent is removed by inducing a phase transition in the embedding agent and separating the embedding agent from the sample; and c) extracting an analyte from the de-embedded sample.

Embodiment 181. The method of embodiment 180, wherein the separation of embedding agent from the embedded sample is improved relative to a method wherein the embedding agent is dissolved with a miscible solvent, optionally wherein the miscible solvent is xylene, ethyl acetate, CitriSolv™, or UltraClear™.

Embodiment 182. The method of embodiment 180, wherein the separation of embedding agent from the embedded sample is improved relative to a method wherein the embedding agent is removed by cutting the embedding agent from the embedded sample.

Embodiment 183. The method of any one of embodiments 180-182, wherein the method is performed in a liquid handling robot comprising a filter, wherein the improved separation of embedding agent results in less clogging of the filter relative to a method wherein the embedding agent is dissolved with a miscible solvent, optionally wherein the miscible solvent is xylene, ethyl acetate, CitriSolv™, or UltraClear™.

Embodiment 184. The method of any one of embodiments 180-183, wherein the method is performed in a liquid handling robot comprising a filter, wherein the improved separation of embedding agent results in less clogging of the filter relative to a method wherein the embedding agent is removed by cutting the embedding agent from the embedded sample.

Embodiment 185. The method of embodiment 183 or embodiment 184, wherein the filter is a filter in a spin column.

Embodiment 186. The method of any one of embodiments 170-184, wherein step b) does not comprise dissolving the embedding agent with a miscible solvent, optionally wherein the miscible solvent is xylene, ethyl acetate, CitriSolv™, or UltraClear™.

Embodiment 187. The method of any one of embodiments 170-186, wherein the phase transition is selected from the group consisting of melting, freezing, vaporization, condensation, sublimation, and deposition.

Embodiment 188. The method of any one of embodiments 170-187, wherein the phase transition is melting.

Embodiment 189. The method of any one of embodiments 170-188, wherein step b) comprises heating, cooling, increasing the pressure, or decreasing the pressure of the embedded sample.

Embodiment 190. The method of any one of embodiments 170-189, wherein step b) comprises heating the embedded sample.

Embodiment 191. The method of embodiment 190, wherein the embedded sample is heated to about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., or about 75° C.

Embodiment 192. The method of embodiment 190 or embodiment 191, wherein the embedded sample is heated for about 5 minutes, about 10 minutes, about 15 minutes, about 20 minutes, about 25 minutes, about 30 minutes, or about 35 minutes.

Embodiment 193. The method of any one of embodiments 170-192, wherein step b) comprises centrifuging and filtering the embedded sample to separate the embedding from the sample.

Embodiment 194. The method of embodiment 193, wherein the embedded sample is centrifuged at about 1,700 rcf, about 1,750 rcf, about 1,800 rcf, about 1,850 rcf, or about 1,900 ref.

Embodiment 195. The method of embodiment 193 or embodiment 194, wherein the embedded sample is centrifuged at 1,811 rcf or greater.

Embodiment 196. The method of any one of embodiments 193-195, wherein the embedded sample is centrifuged at 1,811 rcf.

Embodiment 197. The method of any one of embodiments 170-196, wherein step b) comprises heating, centrifuging, and filtering the embedded sample to separate the embedding agent from the sample, thereby generating a de-embedded sample.

Embodiment 198. The method of any one of embodiments 170-196, wherein step b) comprises heating and centrifuging the embedded sample to separate the embedding agent from the sample, thereby generating a de-embedded sample.

Embodiment 199. A method of detecting an analyte, wherein the method comprises: a) providing an embedded sample comprising an analyte, wherein the sample is embedded in an embedding agent; b) removing the embedding agent from the embedded sample, thereby generating a de-embedded sample, wherein the embedding agent is removed by contacting the embedded sample with an immiscible solvent and separating the embedding agent from the sample; c) extracting the analyte from the de-embedded sample; and d) analyzing the analyte to detect the analyte.

Embodiment 200. The method of embodiment 199, wherein the detection of the analyte is improved relative to a method wherein the embedding agent is dissolved with a miscible solvent, optionally wherein the miscible solvent is xylene, ethyl acetate, CitriSolv™ or UltraClear™.

Embodiment 201. The method of embodiment 199, wherein the detection of the analyte is improved relative to a method wherein the embedding agent is removed by cutting the embedding agent from the embedded sample.

Embodiment 202. The method of embodiment 199, wherein the detection the analyte is improved relative to a method wherein the embedded sample is not de-embedded.

Embodiment 203. A method of extracting an analyte, wherein the method comprises: a) providing an embedded sample comprising an analyte, wherein the sample is embedded in an embedding agent; b) removing the embedding agent from the embedded sample, thereby generating a de-embedded sample, wherein the embedding agent is removed by contacting the embedded sample with an immiscible solvent and separating the embedding agent from the sample; and c) extracting the analyte from the de-embedded sample.

Embodiment 204. The method of embodiment 203, wherein the extraction of the analyte is improved relative to a method wherein the embedding agent is dissolved with a miscible solvent, optionally wherein the miscible solvent is xylene, ethyl acetate, CitriSolv™ or UltraClear™.

Embodiment 205. The method of embodiment 203, wherein the extraction of the analyte is improved relative to a method wherein the embedding agent is removed by cutting the embedding agent from the embedded sample.

Embodiment 206. A method of reducing the level of embedding agent in an analyte sample extracted from an embedded sample, wherein the method comprises: a) providing an embedded sample comprising an analyte, wherein the sample is embedded in an embedding agent; b) removing the embedding agent from the embedded sample, thereby generating a de-embedded sample, wherein the embedding agent is removed by contacting the embedded sample with an immiscible solvent and separating the embedding agent from the sample; c) extracting the analyte from the de-embedded sample; and d) purifying the extracted analyte to provide an analyte sample.

Embodiment 207. The method of embodiment 206, wherein the level of embedding agent in the analyte sample is reduced relative to a method wherein the embedding agent is dissolved with a miscible solvent, optionally wherein the miscible solvent is xylene, ethyl acetate, CitriSolv™, or UltraClear™.

Embodiment 208. The method of embodiment 206, wherein the level of embedding agent in the analyte sample is reduced relative to a method wherein the embedding agent is removed by cutting the embedding agent from the embedded sample.

Embodiment 209. A method of improving separation of embedding agent from an embedded sample, wherein the method comprises: a) providing an embedded sample comprising an analyte, wherein the sample is embedded in an embedding agent; b) removing the embedding agent from the embedded sample, thereby generating a de-embedded sample, wherein the embedding agent is removed by contacting the embedded sample with an immiscible solvent and separating the embedding agent from the sample; and c) extracting the analyte from the de-embedded sample.

Embodiment 210. The method of embodiment 209, wherein the separation of embedding agent from the embedded sample is improved relative to a method wherein the embedding agent is dissolved with a miscible solvent, optionally wherein the miscible solvent is xylene, ethyl acetate, CitriSolv™, or UltraClear™.

Embodiment 211. The method of embodiment 209, wherein the separation of embedding agent from the embedded sample is improved relative to a method wherein the embedding agent is removed by cutting the embedding agent from the embedded sample.

Embodiment 212. The method of any one of embodiments 209-211, wherein the method is performed in a liquid handling robot comprising a filter, wherein the improved separation of embedding agent results in less clogging of the filter relative to a method wherein the embedding agent is dissolved with a miscible solvent, optionally wherein the miscible solvent is xylene, ethyl acetate, CitriSolv™, or UltraClear™.

Embodiment 213. The method of any one of embodiments 209-212, wherein the method is performed in a liquid handling robot comprising a filter, wherein the improved separation of embedding agent results in less clogging of the filter relative to a method wherein the embedding agent is removed by cutting the embedding agent from the embedded sample.

Embodiment 214. The method of embodiment 212 or embodiment 213, wherein the filter is a filter in a spin column.

Embodiment 215. The method of any one of embodiments 199-213, wherein step b) does not comprise dissolving the embedding agent with a miscible solvent, optionally wherein the miscible solvent is xylene, ethyl acetate, CitriSolv™, or UltraClear™.

Embodiment 216. The method of any one of embodiments 199-215, wherein the density of the immiscible solvent is lighter than water and heavier than the embedding agent when the embedding agent is in liquid form.

Embodiment 217. The method of embodiment 216, wherein the density of the immiscible solvent is heavier than liquid paraffin.

Embodiment 218. The method of any one of embodiments 199-217, wherein the immiscible solvent is vegetable oil.

Embodiment 219. The method of any one of embodiments 199-217, wherein the immiscible solvent is mineral oil.

Embodiment 220. The method of embodiment 219, wherein the embedded sample is contacted with about 300, 350, 400, 450, 500, or 550 μL of mineral oil.

Embodiment 221. The method of embodiment 219 or embodiment 220, wherein the embedded sample is contacted with mineral oil at a mineral oil to embedded sample ratio of about 1:4, 1:2, 1:1, 2:1, 4:1, 5:1, 6:1, 7:1, 8:1, 9:1, 10:1, 15:1, 20:1, 30:1, 40:1, or 50:1.

Embodiment 222. The method of any one of embodiments 219-221, wherein the mineral oil contacts the embedded sample at about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., or about 75° C.

Embodiment 223. The method of any one of embodiments 219-222, wherein the mineral oil contacts the embedded sample for about 5 minutes, about 10 minutes, about 15 minutes, about 20 minutes, about 25 minutes, about 30 minutes, or about 35 minutes.

Embodiment 224. The method of any one of embodiments 219-223, wherein the mineral oil contacts the embedded sample while shaking at about 25 rpm, about 50 rpm, about 100 rpm, about 200 rpm, about 300 rpm, about 400 rpm, about 500 rpm, about 600 rpm, about 700 rpm, about 800 rpm, about 900 rpm, about 1000 rpm, or about 1100 rpm.

Embodiment 225. The method of any one of embodiments 199-224, wherein step b) comprises contacting the sample with an immiscible solvent, centrifuging, and filtering the embedded sample.

Embodiment 226. The method of embodiment 225, wherein the embedded sample is centrifuged at about 1,700 rcf, about 1,750 rcf, about 1,800 rcf, about 1,850 rcf, or about 1,900 rcf.

Embodiment 227. The method of embodiment 225 or embodiment 226, wherein the embedded sample is centrifuged at 1,811 rcf or greater.

Embodiment 228. The method of any one of embodiments 225-227, wherein the embedded sample is centrifuged at 1,811 rcf.

Embodiment 229. The method of any one of embodiments 170-228, wherein the analyte is selected from the group consisting of a polypeptide, RNA, DNA, a small molecule, a lipid, a polysaccharide, an exosome, a mitochondria, and a nucleus.

Embodiment 230. The method of any one of embodiments 170-229, wherein the embedding agent is selected from the group consisting of paraffin, resin, celloidin, Paraplast®, gelatin, ester wax, wax, polyethylene glycol, and nitrocellulose.

Embodiment 232. A method of extracting RNA from a paraffin-embedded sample, wherein the method comprises: a) incubating the sample with a protease; b) incubating the sample at 50° C. to 75° C. for 1 to 40 minutes; c) centrifuging the sample at a high speed to separate the paraffin from a lysate comprising the RNA; and d) aspirating the lysate comprising the RNA.

Embodiment 233. The method of embodiment 232, further comprising cooling the sample to room temperature after step c) and before step d).

Embodiment 234. The method of embodiment 233, further comprising centrifuging the sample to filter the lysate following cooling the sample to room temperature.

Embodiment 235. The method of any one of embodiments 232-234, wherein incubating the sample with the protease comprises incubation at from 50° C. to 60° C.

Embodiment 236. The method of any one of embodiments 232-235, wherein incubating the sample with the protease comprises incubation for 1-20 minutes.

Embodiment 237. The method of any one of embodiments 232-236, wherein centrifuging the sample at high speed comprises centrifuging the sample at about 1,700 rcf, about 1,750 rcf, about 1,800 rcf, about 1,850 rcf, or about 1,900 rcf.

Embodiment 238. The method of any one of embodiments 232-237, wherein the RNA is isolated from the lysate.

Embodiment 239. The method of any one of embodiments 232-238, wherein the method of extracting RNA is used in a high throughput method.

Embodiment 240. The method of any one of embodiments 232-239, further comprising analyzing the RNA.

Embodiment 241. The method of any one of embodiments 232-240, wherein the incubation with the protease is performed with shaking between 500 and 2000 rpm.

Embodiment 242. The method of any one of embodiments 232-241, wherein the incubation at 50° C. to 80° C. is performed without shaking.

Embodiment 243. The method of any one of embodiments 232-242 further comprising centrifuging the sample at from 250 RCF to 750 RCF following step c).

Embodiment 244. The method of any one of embodiments 232, further comprising preparing cDNA from the RNA.

Embodiment 245. A method of extracting DNA from a sample, wherein the method comprises: a) incubating the sample with a protease; b) incubating the sample at 50° C. to 80° C. for 2-48 hours; c) centrifuging the sample at high speed to produce a lysate comprising the DNA; and d) aspirating the lysate comprising the DNA.

Embodiment 246. The method of embodiment 245, wherein incubating the sample at 50° C. to 80° C. for 2-48 hours is performed while shaking at between 500 and 2000 rpm.

Embodiment 247. The method of embodiment 245 or 246, wherein immediately following step b), the sample is centrifuged for 2 to 20 minutes at from 1000 RCF to 4000 RCF.

Embodiment 248. The method of any embodiment 247, wherein the sample is refrigerated for at least 40 minutes following centrifugation for 2 to 20 minutes at from 1000 RCF to 4000 RCF.

Embodiment 249. The method of embodiment 248, wherein step c) is carried out after refrigerating the sample.

Embodiment 250. The method of any one of embodiments 245-249, wherein centrifuging the sample at high speed comprises centrifuging the sample at about 1,700 rcf, about 1,750 rcf, about 1,800 rcf, about 1,850 rcf, or about 1,900 rcf.

Embodiment 251. A method of extracting RNA and DNA from a paraffin-embedded sample, wherein the method comprises: a) incubating the sample with a protease; b) incubating the sample at 50° C. to 80° C. for 1 to 40 minutes; c) centrifuging the sample at a high speed to separate the paraffin from a first lysate comprising the RNA; d) aspirating the first lysate comprising the RNA; e) isolating the RNA from the first lysate; f) incubating the sample from step c) with a protease; g) incubating the sample from step f) at 50° C. to 80° C. for 2-48 hours; h) centrifuging the sample at high speed; i) aspirating a second lysate comprising the DNA; j) isolating the DNA from the second lysate.

Embodiment 252. The method of embodiment 251, wherein centrifuging the sample at high speed comprises centrifuging the sample at about 1,700 rcf, about 1,750 rcf, about 1,800 rcf, about 1,850 rcf, or about 1,900 rcf.

Embodiment 253. The method of embodiments 251 or 252, wherein incubation with the protease is performed while shaking at between 500 and 2000 rpm in step a and/or step f.

Embodiment 254. The method of any one of embodiments 251-253, wherein incubating the sample with the protease comprises incubation at from 50° C. to 60° C.

Embodiment 255. The method of any one of embodiments 251-254, wherein incubating the sample with the protease comprises incubation for 1-20 minutes.

Embodiment 256. The method of any one of embodiments 251-255, wherein incubating the sample at 50° C. to 80° C. for 1 to 40 minutes is performed without shaking.

Embodiment 257. A method of extracting RNA from a paraffin-embedded sample, wherein the method comprises: a) adding mineral oil to the sample; b) incubating the sample at 50° C. to 80° C. for 1 to 40 minutes; c) cooling the sample to room temperature; d) incubating the sample with a protease; f) incubating the sample at 50° C. to 80° C. for 1 to 40 minutes; g) centrifuging the sample at a high speed to separate the paraffin and the mineral oil from a lysate comprising the RNA; and h) aspirating the lysate comprising the RNA.

Embodiment 258. The method of embodiment 257, wherein the incubation at 50° C. to 80° C. for 1 to 20 minutes is performed with shaking at from 500 RPM to 2000 RPM.

Embodiment 259. The method of any one of embodiments 257-258, wherein centrifuging the sample at high speed comprises centrifuging the sample at about 1,700 rcf, about 1,750 rcf, about 1,800 rcf, about 1,850 rcf, or about 1,900 rcf.

Embodiment 260. The method of any one of embodiments 256-259, wherein the sample is incubated at room temperature following centrifugation at high speed.

Embodiment 261. The method of embodiment 260, further comprising centrifuging the sample after incubation at room temperature.

Embodiment 262. A method of extracting DNA from a paraffin-embedded sample, wherein the method comprises: a) adding mineral oil to the sample; b) incubating the sample with a protease; c) incubating the sample at 50° C. to 80° C. for 2-48 hours; d) centrifuging the sample at a high speed to separate the paraffin from a lysate comprising the DNA; and e) aspirating the lysate comprising the DNA.

Embodiment 263. The method of embodiment 262, wherein the mineral oil is removed prior to step b).

Embodiment 264. The method of any one of embodiments 262-263, wherein centrifuging the sample at high speed comprises centrifuging the sample at about 1,700 rcf, about 1,750 rcf, about 1,800 rcf, about 1,850 rcf, or about 1,900 rcf.

Embodiment 265. The method of any one of embodiments 262-264, wherein incubating the sample at 50° C. to 80° C. for 2-48 hours is performed with shaking at from 500 RPM to 2000 RPM.

Embodiment 266. The method of any one of embodiments 262-265, wherein following incubating the sample at 50° C. to 80° C. for 2-48 hours, the sample is immediately centrifuged at 2000 to 5000 RCF prior to step d).

Embodiment 267. A method of extracting RNA and DNA from a paraffin-embedded sample, wherein the method comprises: a) adding mineral oil to the sample; b) melting the paraffin by incubating the sample at 50° C. to 80° C. for 1 to 40 minutes; c) cooling the sample to room temperature; d) incubating the sample with a protease; e) incubating the sample at 50° C. to 80° C. for 1 to 40 minutes; f) centrifuging the sample at a high speed to separate the paraffin and the mineral oil from a lysate comprising the RNA; g) aspirating the lysate comprising the RNA; h) isolating the RNA from the lysate; i) centrifuging the lysate from step g) at a high speed to separate the lysate from the mineral oil; j) incubating the sample from step i) with a protease; k) incubating the sample from step j) at 50° C. to 80° C. for 2-48 hours; 1) centrifuging the sample at high speed; and m) aspirating a second lysate comprising the DNA.

Embodiment 268. The method of embodiment 267, wherein centrifuging the sample at high speed comprises centrifuging the sample at about 1,700 rcf, about 1,750 rcf, about 1,800 rcf, about 1,850 rcf, or about 1,900 rcf.

Embodiment 269. The method of any one of embodiments 267-268, wherein incubating the sample at 50° C. to 80° C. for 2-48 hours is performed with shaking at from 500 RPM to 2000 RPM.

Embodiment 270. The method of any one of embodiments 232-269, wherein the protease is proteinase K.

Embodiment 271. The method of any one of embodiments 1-270, wherein the sample is less than about 30 μm3 in size.

Embodiment 272. The method of any one of embodiments 1-271, wherein the sample is about 0.3 μm3 to about 5.5 μm3 in size.

Embodiment 273. The method of any one of embodiments 232-272, wherein the method further comprises analyzing the RNA and/or the DNA extracted from the sample by one or more methods selected from the group consisting of a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), and mass-spectrometric genotyping.

Embodiment 274. The method of embodiment 273, wherein analyzing the RNA and/or DNA comprises next generation sequencing.

Embodiment 275. The method of any one of embodiments 232-274, further comprising preparing a sequencing library for sequencing the RNA and/or the DNA.

Embodiment 276. The method of any one of embodiments 232-275, further comprising sequencing the DNA and/or RNA using hybrid capture based sequencing.

Embodiment 277. The method of any one of embodiments 232-276, wherein the sample is from an individual known to have cancer or suspected of having cancer.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1. Co-Extraction Heat/Creaming “Warm Lysate Spin” Method

This example describes RNA and/or DNA co-extraction as outlined in FIG. 3A (right column) and FIG. 3B (right column), termed the “warm lysis spin,” the “warm lysate method,” or the “warm lysate process.” The method may be compared to the “standard lysis” process outlined in FIG. 3A (left column) and FIG. 3B (left column).

1A. Protocols for RNA AutoLys co-extraction and purification. Reagents are obtained from Omega MAG-BIND® FFPE RNA Kit (Omega Bio-tek, Norcross, Georgia, USA; catalogue number M2551), including RML buffer, proteinase K solution, MFB buffer, MAG-BIND® particles SC, RNA wash buffer II, GFC buffer, DNase digestion buffer, and MAG-BIND® DNase I.

(1) Formalin-fixed paraffin-embedded (FFPE) curls are placed into AutoLys tubes. Up to 200 μm FFPE paraffined curls can be extracted in a single tube. For cytosmears, the coverslip is removed (as in the method described in Example 3, below) and tissue is scraped into the AutoLys tube. (2) The AutoLys tubes are capped and it is verified that the AutoLys tubes are in the “down” position (with the inner tube fully lowered into the outer tube) before proceeding to tissue digestion. (3) The tubes are arrayed into an AutoLys rack. (4) The rack containing the AutoLys tubes is centrifuged for 3 minutes at 3,000 rpm to pull tissue away from the top of tube. (5) An RNA digestion mix is prepared by combining 315 μL of Omega RML buffer and 35 μL of Omega Proteinase Solution. 350 μL total mix is used per sample. The mix is inverted at least 15 times to mix. (6) The tubes are uncapped at 350 μL of the RNA digestion mix is pipetted into each tube. The tubes are recapped with the lid. (7) The rack containing the AutoLys tubes is incubated using a VorTemp incubator for 15 minutes at 56° C. (the VorTemp is preheated to 56° C.) with 1,000 RPM shaking. (8) After step (7), the rack is moved to a different VorTemp preheated to 70° C. and incubated for 5 minutes with no shaking. This step ensures all of the paraffin has melted, leaving an emulsion. (9) Once the incubation of (8) is complete, the rack is removed and immediately the samples are centrifuged for 3 minutes at 1,811 RCF with inner tubes in lower position. This step ensures phase separation and allows for the melted paraffin to float to the top of the liquid in the AutoLys tube. (10) After centrifugation, the tube rack is removed from the centrifuge and cooled for 5 minutes at room temperature. This step allows the melted paraffin to form a ring at the top of the liquid column. (11) The rack containing tubes is removed from the centrifuge. (12) The inner AutoLys tubes are lifted and locked and returned to the rack. (13) The rack is centrifuged for 2 minutes at 500 RCF. (14) Using a pipette, the lysate is aspirated from collection tubes and dispensed into an empty 96 deep-well KingFisher block labeled “Lysate.” If samples are not proceeding immediately to DNA digestion, the AutoLys tubes with inner tubes returned to their bottom position are stored at 4° C. for up to 20 hours. (15) The 96 deep-well KingFisher block is sealed (using MicroAMP Clear adhesive film) for immediate de-crosslinking. (16) The KingFisher block is placed on a VorTemp. (17) The RNA is de-crosslinked at 80° C. for 60 minutes with 1,000 RPM shaking. (18) During the incubation, the KingFisher reagent plates are prepared. (19) When the de-crosslinking is complete, the KingFisher block is placed on ice for at least 5 minutes to cool the lysate. (20) The process then proceeds directly to RNA Extraction on KingFisher.

(21) Omega MAG-BIND® Particles SC are removed from their 4° C. storage. (22) The cooled RNA lysate plate is centrifuged for 1 minute at 2,000 RPM. (23) The MFB Buffer and MAG-BEAD® Master Mix is prepared. Before adding the MAG-BIND Particles SC, the bottle is inverted 10 times and vortexed for 10 seconds to ensure the bead solution is fully homogenized. If beads are not fully homogenized after 10 seconds, the mix is repeated as needed. (24) 350 μL Omega MFB Buffer, 35 μL Mag beads SC, and 650 μL 100% ethanol are added to wells of lysate block. The solution is mixed 15-20 times using a pipette. (25) The solution is split by pipetting half of the volume into a second 96 deep-well KingFisher block labeled “Lysate 2”. [0557](26) The remaining reagent blocks are labeled, prepared, and briefly centrifuged as follows: (27) “Wash 1” 800 μL RNA Wash Buffer II, deep well; (28) “Wash 2” 400 μL RNA Wash Buffer II; (29) “Wash 3” 400 μL RNA Wash Buffer II, deep well; (30) “DNase mixture” 73.5 μL DNase Buffer with 1.5 μL DNase, deep well; (31) KingFisher Tip Comb, deep well (no need to centrifuge); (32) “Elution” 65 μL DEPC water, KingFisher 96 KF microplate (200 μL).

(33) The KingFisher blocks are loaded in the KingFisher machine and the KingFisher RNA extraction is run using a custom protocol.

(34) Once complete, RNA eluate from microplate is transferred into individual tubes and stored at −80° C. If samples are not proceeding immediately to DNA Digestion, AL-racks may be stored at 4° C. for up to 20 hours.

1B Protocols for DNA AutoLys co-extraction and purification. Reagents used include Promega Incubation Buffer, Promega Proteinase K (ProK) Lyophilized, nuclease-free water, Omega DNA Wash Buffer, ethyl alcohol 200 proof, isopropyl alcohol, Omega MAG-BIND® Particles CH, Omega MB4 Buffer, Omega MPW Buffer, and 1×Tris-EDTA buffer (pH 8.0).

(35) AutoLys tubes are removed from 4° C. storage. The tubes are centrifuged for 2 minutes at 800 RCF to pull tissue and/or condensation away from the top of the tube. (36) Master mix is prepared with 315 μL Promega Incubation Buffer and 35 μL of Promega ProK (after hydrating a new proteinase K with 5 mL nuclease free water). 350 μL is pipetted into each AutoLys tube with the inner tube in the lower position. (37) The AutoLys tubes are incubated for 8-20 hours at 70° C. with 1,000 RPM shaking in a VorTemp. (38) After the VorTemp incubation, the rack(s) are removed from the VorTemp and immediately centrifuged for 10 minutes at 3,000 RCF. (39) The rack(s) are removed from the centrifuge and stored in a 4° C. refrigerator for at least 40 minutes. (40) The AutoLys tubes are lifted and locked and returned to the rack(s). (41) The racks are centrifuged for 2 minutes at 1,811 RCF. (42) Using a pipette, the lysate is aspirated from the collection tube and dispensed into an empty 96 deep-well KingFisher block labeled “Lysate”. Then proceed directly to DNA purification on the KingFisher. (43) 50 μL of MagBind CH beads are added. (44) 875 μL MB4 buffer is added to the 50 μL MagBind Beads and mixed 15-20×using a pipette. The solution is split by pipetting half of the volume (about 640 μL) into second 96 deep-well Kingfisher block labeled “Lysate 2”.

(45) Remaining reagent blocks are labeled, prepared, and briefly centrifuged as follows: 1. “Wash 1” 800 μL DNA Wash Buffer (Omega DNA FFPE Kit), deep-well; 2. “Wash 2” 400 μL DNA Wash Buffer (Omega DNA FFPE kit), deep-well; 3. “MPW” 400 μL MPW Buffer (Omega DNA FFPE Kit), deep-well; 4. KingFisher Tip Comb, deep-well (no need to centrifuge); and 5. “Elution” 75 μL 1×TE, KingFisher 96 KF microplate (200 μL).

(46) A custom protocol is used on the KingFisher, the blocks are loaded, and the program is run. Once complete, the DNA eluate from the microplate is transferred into matrix tubes and stored at 20° C.

### Example 2. Co-Extraction Mineral Oil Methodology (FFPE Tissue)

This example describes RNA and/or DNA co-extraction using an oil method, as outlined in FIG. 3A (middle column) and FIG. 3B (middle column).

A. Protocols for RNA AutoLys co-extraction and purification. (1) FFPE 10-200 μm curls are plated into AutoLys tubes. Up to 200 μm FFPE paraffined curls can be extracted in a single tube. (2) The AutoLys tubes are capped and the AutoLys tube is confirmed in the “down” position (with the inner tube fully lowered into the outer tube) before proceeding to tissue digestion. (3) The tubes are arrayed in a AutoLys rack. (4) The rack containing the AutoLys tubes is centrifuged for 2 minutes at 800 RCF to pull tissue away from the top of the tube. (5) The tubes are uncapped. (6) 300 μL mineral oil is added to each tube. (7) The tubes are recapped. (8) The rack containing the AutoLys tubes is incubated using a preheated VorTemp for 20 minutes at 65° C. with 1,000 rpm shaking to melt the paraffin. This step ensures that all paraffin has melted. (9) When incubation is complete, the rack is removed from the VorTemp and incubated at room temperature for 10 minutes. (10) While the sample is cooling off, the RNA digestion mix is prepared by combining 315 μL of Omega RML Buffer and 35 μL of Omega Proteinase Solution. 350 μL RNA digestion mix is prepared per sample. The RNA digestion mix is inverted at least 15 times to mix, but not vortexed. (11) The tubes are uncapped and 350 μL of RNA digestion mix is pipetted into each tube. The tubes are capped with their lids. (12) The rack is spun at 1,811 RCF for 2 minutes. (13) The rack containing the AutoLys tubes is incubated using the VorTemp for 15 minutes at 56° C. with 1,000 rpm shaking. (14) Once the incubation is completed, the rack containing the AutoLys tubes is removed and immediately proceed to centrifuge the samples for 8 minutes at 1,811 RCF with inner tube in lower position. This step allows for phase separation and for the melted paraffin and oil to float to the top of the liquid in the AutoLys tube. (15) The tubes are left for 5 minutes at room temperature. (16) The inner AutoLys tubes are lifted and locked and the tubes are returned to the rack. (17) The racks are centrifuged for 2 minutes at 500 RCF. (18) Using a pipette, the lysate is aspirated from the collection tube and dispensed into empty 96 deep-well Kingfisher block labeled “Lysate”. If samples are not proceeding immediately to DNA Digestion, the AutoLys tubes with inner tubes returned to their bottom position can be stored at 4° C. for up to 20 hours. (19) The 96 deep-well KingFisher block containing the RNA lysate is sealed (using MicroAMP Clear adhesive film) and proceeds immediately to the de-crosslinking step. (20) The KingFisher block is placed on the VorTemp. (21) The RNA is de-crosslinked at 80° C. for 60 minutes with 1,000 rpm shaking. (22) During the incubation, Kingfisher reagent plates are prepared. (23) When the de-crosslinking is complete, the KingFisher plate is placed on ice for at least 5 minutes to cool the lysate. (24) RNA Extraction on KingFisher is performed as described in the preceding Example.

B. Protocols for DNA AutoLys co-extraction and purification. (1) AutoLys tubes containing partially digested tissue from RNA extraction are removed from 4° C. storage. (2) The inner tube is lifted and locked. (3) The AutoLys tubed are centrifuged for 2 minutes at 1,811 RCF. (4) The oil is removed from the outer tube. (5) The inner tube is placed in the “down” position (with the inner tube fully lowered into the outer tube) before proceeding to tissue digestion. (6) Master mix is prepared containing 315 μL Promega incubation buffer and 35 μL of Promega ProK (a new ProK is hydrated with 5 mL nuclease free water before use). 350 μL of mix is pipetted into each AutoLys tube with the inner tube in the “down” position. (7) the AutoLys tubes are incubated for 8-20 hours at 70° C. with 1,000 rpm shaking in a VorTemp. (8) Once the centrifuge is ready, the VorTemp incubation is stopped. The rack(s) are removed from the VorTemp and immediately centrifuged for 10 minutes at 3,000 RCF. (9) The rack(s) are removed from the centrifuge and stored in a 4° C. refrigerator for at least 40 minutes. (10) The inner AutoLys tubes are lifted and locked and returned to the rack(s). (11) The rack(s) are centrifuged for 2 minutes at 1,811 RCF. (12) Using a pipette, the lysate is aspirated from the collection tubes and dispensed into an empty 96 deep-well KingFisher block labeled “Lysate”. The samples then proceed directly to DNA purification on the KingFisher.

(13) 50 μL MAG-BIND® CH beads are added. (14) 875 μL MB4 buffer (Omega DNA FFPE Kit) is added to 50 μL MAG-BIND beads and the solution is mixed 15-20×using a pipette. The solution is split by pipetting half of the volume (about 640 μL) into a second 96 deep-well KingFisher block labeled “Lysate 2”.

(15) Remaining reagent blocks are labeled, prepared, and briefly centrifuged as follows: i. “Wash 1” 800 μL DNA Wash Buffer (Omega DNA FFPE Kit), deep-well; ii. “Wash 2” 400 μL DNA Wash Buffer (Omega DNA FFPE Kit), deep-well; iii. “MPW” 400 μL MPW Buffer (Omega DNA FFPE Kit), deep-well; iv. KingFisher Tip Comb, deep-well (no need to centrifuge); v. “Elution” 75 μL 1×TE, KingFisher 96 KF microplate (200 μL).

(16) The KingFisher is run using a custom program. Once complete, the DNA eluate is transferred into matrix tubes and stored at 20° C.

### Example 3. De-Coverslipping Method

(1) A Coplin jar is filled with fresh xylene. The slide to be de-coverslipped is inserted in the jar and submerged in the solution for at least 30 minutes for the coverslip to fall off. (2) The slide without coverslip is re-submerged in xylene for an additional 30 minutes to remove any residual mounting media from the specimen surface. (3) The slide is removed from the jar and laid on a fresh, clean absorbent pad. (4) The Coplin jar is emptied and filled with 95% EtOH to remove any residual mounting media/xylene. (5) The jar is incubated for 15-30 seconds and then the solution is discarded. (6) Fresh 95% EtOH is added to the jar and the slide is immersed. The material is allowed to incubate for at least 5 minutes but no more than 15 minutes. (7) The slide is removed from the jar and placed on a clean KimWipe, with sample side up. (8) The slide is covered with a second KimWipe. (9) The tissue is scraped into an AutoLys tube for RNA/DNA co-extraction.

### Example 4. Comparison of Standard and Warm Lysate Process

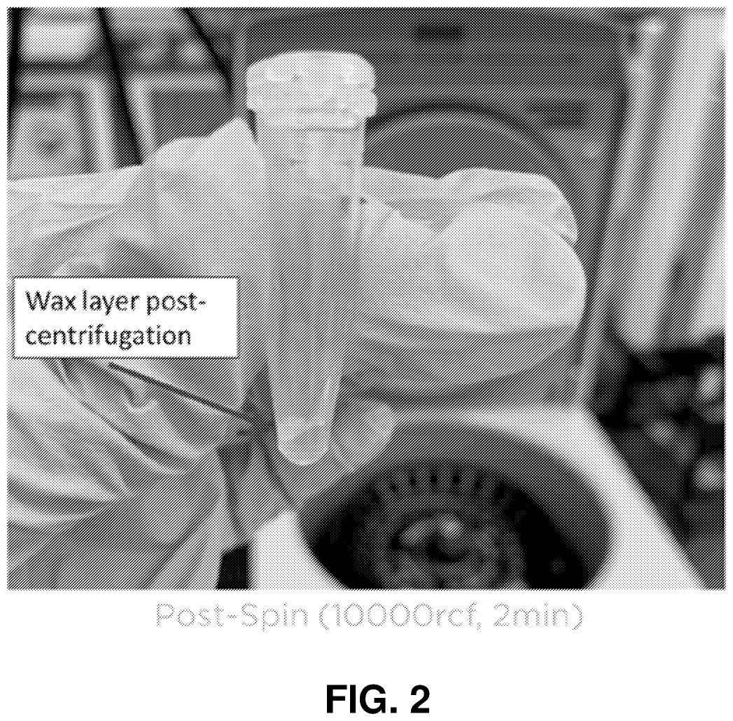

The standard AutoLys method was found to produce a wax layer post-centrifugation (see FIG. 2 and description of process in left column of FIG. 3A and left column of FIG. 3B). The standard method was compared with the warm lysate method described in Example 1, above, and as outlined in the right column of FIG. 3A and right column of FIG. 3B.

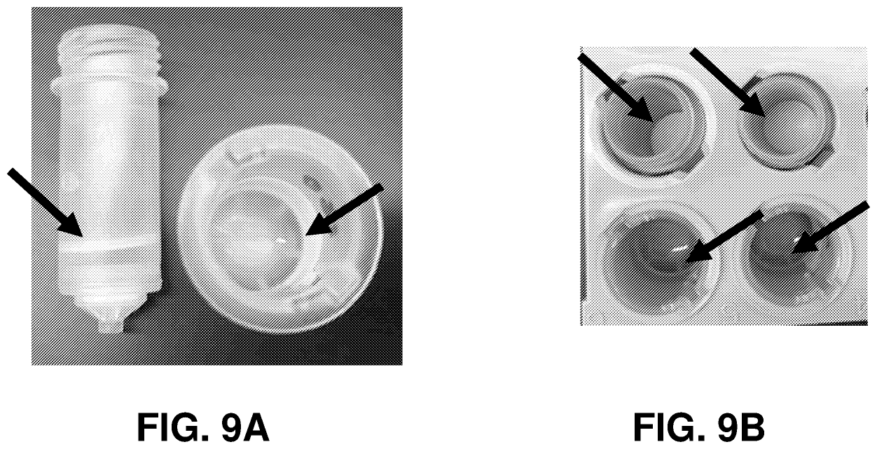

As shown in FIG. 9A and FIG. 9B, the warm lysate process produced a paraffin layer that remains in the inner tube (indicated by the white arrows in both FIG. 9A and FIG. 9B; compare to standard process results in FIG. 2). The warm lysate process left a clear eluate with minimal residual paraffin in the lysate collection tubes (indicated by black arrows in both FIG. 9A and FIG. 9B). The results show that the improved warm lysate process can effectively separate the paraffin portion from the lysate portion, in contrast to the standard process (as shown in FIG. 2) which leaves a wax (paraffin) layer post-centrifugation.

RNA Lab Metrics were collected for RNA prepared by the standard process (arm 2), the warm lysate process (arm 4), and for a baseline extraction method (arm 6, a manual RNA extraction process; which is not automated like the AutoLys processes). The results are shown in Table 1, below.

TABLE 1
RNA Lab Metrics Summary for Standard vs. Warm Lysate Extraction

| | | Median | RNAx | Median | LC Pass | Median | HC Pass |
|---|---|---|---|---|---|---|---|
| | Clogging | RNA | Pass Rate | LC Yield | Rate | HC Yield | Rate |
| Process | (%) | yield (ng) | (>192 ng) | (ng) | (>750 ng) | (ng) | (>0.1 ng) |
| Manual | N/A | 2,099 | 95.55% | 3,811 | 93% | 139.31 | 100% |
| Arm 6 | | | (86/90) | (N = 86) | (80/86) | | (80/80) |
| Standard | 9.4% | 5,971 | 98.9% | 2,255 | 74.74% | 118.82 | 100% |
| Arm 2 | | | (95/96) | (N = 95) | (71/95) | | (71/71) |
| Warm | 0% | 7,789 | 98.9% | 2,957 | 85.26% | 134.36 | 100% |
| Lysate | | | (95/96) | (N = 95) | (81/95) | | (81/81) |
| Arm 4 | | | | | | | |

The percent clogging is a measure of how often the inner AutoLys tube was clogged by paraffin, which impairs the recovery of sufficient lysate. RNA extraction (RNAx) is a measure of how often >192 ng of RNA is collected. Library construction (LC) pass rate is a measure of how often a cDNA library with >750 ng cDNA is generated. Hybrid Capture (HC) is a measure of how often a capture library with >0.1 ng DNA for sequencing is obtained. The warm lysate process demonstrated improvements over the standard process with significant improvement in clogging (0% versus 9.4%) and improved LC pass rate (85.26% versus 74.74%). Further, compared to the manual extraction process, the warm lysate process is automated and had significantly improved overall RNA yield. The difference in LC pass rate between the manual extraction and warm lysate process was not statistically significant.

RNA Sequencing Metrics were collected, as well, for RNA prepared by the standard process, the warm lysate process, and by the baseline manual RNA extraction process. The results are shown in Table 2, below.

TABLE 2
RNA Sequencing Metrics Summary for Standard vs. Warm Lysate Extraction

| | | | | On-target | | Insert | |
|---|---|---|---|---|---|---|---|
| | | | Median | Distinct | | Size | |
| | Pre-dup | On-target | On-Target | Pairs Pass | Median | Pass | |
| | % | % | Distinct | Rate (>3 | Insert | Rate | % |
| Process | Selected | duplication | Pairs | million) | Size | (>70 nt) | Chimeras |
| Manual Arm 6 | 94.39 | 71.86 | 5,543,808 | 87.5% | 76.64 | 73.7% | 0.058 |
| | | | | (70/80) | | (59/80) | |
| Standard Arm 2 | 94.90 | 75.36 | 5,126,311 | 87.32% | 74.64 | 73.24% | 0.059 |
| | | | | (62/71) | | (52/71) | |
| Warm | 94.86 | 75.30 | 5,461,822 | 82.72% | 76.44 | 71.60% | 0.053 |
| Lysate | | | | (67/81) | | (58/81) | |
| Arm 4 | | | | | | | |

Pre-dup % Selected is the percent of reads mapping to the regions targeted by hybrid capture baits. On-target % duplication is the percent of on-target distinct pairs. On-target distinct pairs pass rate is the percent of distinct pairs with >3 million. Median insert size is the size of nucleic acid fragments between the adapters. Insert size pass rate is the percent of inserts with >70 nt in length. % Chimeras is the percentage of read chimeras. Sequencing metrics performance (pre-dup % selected, on target % selected, median on-target distinct pairs, on-target distinct pairs pass rate, median insert size, insert size pass rate, and % chimeras) was comparable across all processes. These data indicate that the warm lysate process maintains high performance for sequencing metrics compared to the manual process extraction method.

The ability to detect known fusions from RNA extracted by each process was assessed.

TABLE 3
Fusion Detection comparison between
Standard vs. Warm Lysate Extraction

| Process | ALK-EML4 | RET-CCDC6 | ROS-SLC34A2 |
|---|---|---|---|
| Manual | 71 | 240 | 240 |
| Arm 6 | | | |
| Standard | 99 | 560 | 493 |
| Arm 2 | | | |
| Warm Lysate | 120 | 681 | 433 |
| Arm 4 | | | |

Table 3 shows assessment of the ability to detect known fusions from RNA extracted by each process. Three known fusions, ALK-EML4, RET-CCDC6, and ROS-SLC34A2, were assessed. The numbers for each result indicate the number reads detected supporting each fusion. The warm lysate process was able to detect fusions at comparable levels to the standard method and was able to detect fusions at a higher level than the manual RNA extraction.

DNA Lab Metrics were collected for DNA prepared by the standard process (arm 2), the warm lysate process (arm 4), and by a manual DNA extraction process (arm 1, which was performed in replicate, and which is not automated like the AutoLys processes). The results are shown in Table 4, below.

TABLE 4
DNA Lab Metrics Summary for Standard vs. Warm Lysate Extraction

| | Percent | | | | | | |
|---|---|---|---|---|---|---|---|
| | Clogging | Median | | | | | |
| | (<250 uL | DNA | DNAx | Median | LC Pass | Median | HC Pass |
| | Lysate | Yield | Pass Rate | LC Yield | Rate | HC Yield | Rate |
| Process | recovered) | (ng) | (>55 ng) | (ng) | (>545 ng) | (ng) | (>140 ng) |
| Manual | N/A | 4,370 | 100% | 7,085 | 100% | 330.74 | 95.8% |
| | | | (96/96) | | (96/96) | | (92/96) |
| Manual | N/A | 3,491 | 100% | 7,188 | 98.9% | 320.3 | 96.8% |
| | | | (96/96) | | (95/96) | | (92/95) |
| Standard | 2% | 3,289 | 98.96% | 7,262 | 98.94% | 342.72 | 93.6% |
| | | | (95/96) | | (94/95) | | (88/94) |
| Warm | 0% | 2,740 | 100% | 6,600 | 100% | 357.9 | 92.7% |
| Lysate | | | (96/96) | | (96/96) | | (89/96) |

% clogging is a measure of how often the inner AutoLys tube was clogged by paraffin, which impairs recovery such that <250 μL of eluate is recovered. DNA extraction (DNAx) pass rate is a measure of how often >55 ng of DNA is collected. Library Construction (LC) pass rate is a measure of how often a DNA library with >545 ng DNA is generated. Hybrid Capture (HC) pass rate is a measure of how often a captured library with >140 ng DNA for sequencing is obtained. The warm lysate process demonstrated improvements over the standard process, with improvement in clogging (0% versus 2%). Further, the warm lysate process had a high sample success rate (>90% for each category) and did not perform significantly different (DNAx pass rate, LC pass rate, and HC pass rate) than the two manual extraction experiments.

DNA Sequencing Metrics were collected, as well, for DNA prepared by the standard process, the warm lysate process, and by the manual DNA extraction processes. The results are shown in Table 5, below.

TABLE 5
DNA Sequencing Metrics Summary for Standard vs. Warm Lysate Process

| Process | Manual | Manual | Standard | Warm Lysate |
|---|---|---|---|---|
| Pre-dup % | 70.64 | 71.09 | 70.29 | 70.79 |
| Selected | | | | |
| Median Insert Size | 135 | 134 | 139 | 138 |
| % Chimeras | 0.738 | 0.698 | 0.825 | 0.696 |
| Median Exon | 840 | 828 | 844 | 860 |
| Coverage | | | | |
| Exon Coverage | 97.83% (90/92) | 98.91% (91/92) | 98.86% (87/88) | 100% (89/89) |
| Pass Rate (>250X) | | | | |
| Exon Coverage | 86.96% (80/92) | 88.04% (81/92) | 93.18% (82/88) | 89.89% (80/89) |
| Pass Rate (>500X) | | | | |
| CNA Noise Score | 15 | 19.5 | 16 | 13 |
| (median) | | | | |
| CNA Noise | 0.22 | 0.23 | 0.22 | 0.21 |
| (median) | | | | |
| CNA Noise >100 | 19.23% (35/182) | | 26.44% (23/87) | 17.24% (15/87) |
| Qualified for CNA | 9.89% (18/182) | | 17.24% (15/87) | 11.36% (10/88) |
| Noise | | | | |

CNA stands for copy number alterations, which are normalized target coverage compared to a process-matched normal reference sample. Pre-dup % selected is the percent of reads mapping to the regions targeted by hybrid capture baits. Median insert size is the size of nucleic acid fragments between the adapters. % chimeras is the percentage of read chimeras. For median exon coverage, sequence coverage is the number of times each base in the exon regions is covered in the sequence data; the average sequence coverage is calculated in each exon, and then the median (i.e., the median exon coverage) of these values is used. Exon coverage pass rate was assessed at >250× coverage and >500 coverage. CNA noise score is a score to measure noise in copy number plots. For “Qualified for CNA noise,” if the GC bias is high or the data are noisy, the CNA calls cannot be resolved because they are within the level of noise; therefore, the CNA is marked as “Qualified” and the non-equivocal amplifications or losses are removed due to noise. The sequencing metrics data indicate that performance (pre-dup % selected, median insert size, % chimeras, CNA noise, and CNA noise score) was comparable across the processes. Sample success rate for coverage (>250×) was greater than 98% for the automated methods (standard and warm lysate). There was lower copy number noise and a lower number of qualified samples for CNA observed with the warm lysate process compared to the standard AutoLys process.

Statistical analyses. Concordance analysis for variant types (substitution, indel, copy number alteration, fusion/rearrangement) was conducted after sample curation per standard SOPs (QSR-LAB-OP-058). Any discordances were investigated to ensure differences were not dependent on curation. Additional binning for CNA concordance analysis was performed. The concordance of variant calls was assessed for the two manual process replicates pair, standard process vs. each manual process replicate and warm lysate vs. each standard process replicate.

Four metrics, positive percent agreement (PPA), negative percent agreement (NPA), positive predictive value (PPV) and negative predictive value (NPV) were used to evaluate the concordance of variant calls by variant classes Short Nucleotide variants (SNV), Copy Number of Alterations (CNA) and Rearrangements (RE) and also complex biomarkers. The concordance of CNA was further evaluated by copy number bins. The four metrics were defined as:

$$PPA = \frac{\# of variants called positive by both the reference and comparing tests}{\# of variants called positive by the reference test}$$

$$PPV = \frac{\# of variants by both the reference and comparing tests}{\# of variants called positive by the comparing test}$$

$$NPA = \frac{\# of variants called negative by both the reference and comparing tests}{\# of variants called negative by the reference test}$$

$$NPV = \frac{\# of variants called negative by both the reference and comparing tests}{\# of variants called negative by the comparing test}$$

To calculate NPA and NPV, a universal set, including all variants detected in any previous AV studies and new variants in the study, was used for each variant class (SNV, RE, CNA).

The results are summarized in Tables 6-10, below.

TABLE 6
Manual process replicate A vs. Manual process replicate B

| Variant Types | | PPA | NPA | PPV | NPV |
|---|---|---|---|---|---|
| SV | Base | 98.93 | 99.998 | 96.98 | 99.999 |
| | Substitution | (834/843) | (1464847/1464873) | (834/860) | (1464847/1464856) |
| | Indels | 96.30 | 99.997 | 93.69 | 99.999 |
| | | (104/108) | (209381/209388) | (104/111) | (209381/209385) |
| | Overall | 98.63 | 99.999 | 96.60 | 99.999 |
| | | (938/951) | (1676076/1676109) | (938/971) | (1676076/1676089) |

TABLE 7
Standard process vs. Manual process replicate A

| Variant Types | | PPA | NPA | PPV | NPV |
|---|---|---|---|---|---|
| SV | Base | 98.77 | 99.99 | 97.81 | 99.99 |
| | Substitution | (805/815) | (1412536/1412554) | (805/823) | (1412536/1412546) |
| | Indels | 96.23 | 99.99 | 96.23 | 99.99 |
| | | (102/106) | (201904/201908) | (102/106) | (201904/201908) |
| | Overall | 98.48 | 99.99 | 97.63 | 99.99 |
| | | (907/921) | (1616222/1616244) | (907/929) | (1616222/1616236) |

TABLE 8
Standard process vs. Manual process replicate B

| Variant Types | | PPA | NPA | PPV | NPV |
|---|---|---|---|---|---|
| SV | Base | 96.91 | 99.99 | 98.15 | 99.99 |
| | Substitution | (847/874) | (1429928/1429944) | (847/863) | (1429928/1429955) |
| | Indels | 92.62 | 99.99 | 96.58 | 99.99 |
| | | (113/122) | (204382/204386) | (113/117) | (204382/204391) |
| | Overall | 96.39 | 99.99 | 97.96 | 99.99 |
| | | (960/996) | (1636114/1636134) | (960/980) | (1636114/1636150) |

TABLE 9
Warm lysate process vs. Manual process replicate A

| Variant Types | | PPA | NPA | PPV | NPV |
|---|---|---|---|---|---|
| SV | Base | 97.70 | 99.99 | 97.93 | 99.99 |
| | Substitution | (806/825) | (1429976/1429993) | (806/823) | (1429976/1429995) |
| | Indels | 96.23 | 99.99 | 98.08 | 99.99 |
| | | (102/106) | (204400/204402) | (102/104) | (204400/204404) |
| | Overall | 97.53 | 99.99 | 97.95 | 99.99 |
| | | (908/931) | (1636180/1636199) | (908/927) | (1636180/1636203) |

TABLE 10
Warm lysate process vs. Manual process replicate B

| Variant Types | | PPA | NPA | PPV | NPV |
|---|---|---|---|---|---|
| SV | Base | 96.17 | 99.99 | 98.27 | 99.99 |
| | Substitution | (854/888) | (1447364/1447379) | (854/869) | (1447364/1447398) |
| | Indels | 91.06 | 99.999 | 95.73 | 99.99 |
| | | (112/123) | (206874/206879) | (112/117) | (206874/206885) |
| | Overall | 95.55 | 99.99 | 97.97 | 99.99 |
| | | (966/1011) | (1656064/1656084) | (966/986) | (1656064/1656109) |

The percentage agreements (PPA and PPV values) for indels and subs (no filters) between DNA extraction by manual process replicates and for standard AutoLys process and warm lysate process with each replicate was found to be higher than 90%. This demonstrates high concordance for SV between the automated extraction methods and the manual DNA extraction method. The NPA and NPV for SNV between the manual DNA extraction replicates and between the automated co-extraction methods and manual DNA extraction method was greater than 99.99%.

TABLE 11
Concordance: Rearrangements (RE)

| Processes | | | | |
|---|---|---|---|---|
| compared | PPA | NPA | PPV | NPV |
| Manual replicate | 81.82 | 99.99 | 81.82 | 99.99 |
| B vs. manual | (18/22) | (51802/51806) | (18/22) | (51802/51806) |
| replicate A | | | | |
| Standard vs. | 90.48 | 99.99 | 90.48 | 99.99 |
| Manual replicate | (19/21) | (49954/49956) | (19/21) | (49954/49956) |
| A | | | | |
| Standard vs. | 85.71 | 99.99 | 85.71 | 99.99 |
| Manual replicate | (18/21) | (50570/50573) | (18/21) | (50570/50573) |
| B | | | | |
| Warm lysate vs. | 90.91 | 99.99 | 83.33 | 99.99 |
| Manual replicate | (20/22) | (50568/50572) | (20/24) | (50568/50570) |
| A | | | | |
| Warm lysate vs. | 81.82 | 99.99 | 75.00 | 99.99 |
| Manual replicate | (18/22) | (51183/51189) | (18/24) | (51183/51187) |
| B | | | | |

As shown in Table 11, above, the rearrangement PPA for the standard extraction process (90.48%) and warm lysate process (90.91%) with manual DNA extraction process replicate A was greater than the PPA between the manual DNA process replicates (81.82%). The PPA for the standard process (85.71%) and warm lysate process (81.82%) with manual DNA extraction replicate B was similar to the PPA between manual DNA extraction replicates (81.82%). The PPV for standard and warm lysate with manual DNA extraction replicates was higher (>83%) than the agreement between manual DNA extraction replicates (81.82%), except for warm lysate agreement with manual DNA extraction replicate B that was found to be 75%. The low percent agreement for one of the warm lysate processes could be attributed to low statistical power due to the small number of rearrangements found in the sample set. NPA and NPV for rearrangements between manual DNA extraction replicates and between the automated co-extraction methods (standard and warm lysate) and manual DNA extraction was greater than 99.99%.

TABLE 12
Concordance: Copy Number Alterations-Manual DNA extraction
replicate B vs. replicate A (non-equivocal, non-VUS CNAs)

| Variant Types | | PPA | NPA | PPV | NPV |
|---|---|---|---|---|---|
| CNA | Amplification | 86.84 | 99.92 | 83.90 | 99.94 |
| | | (99/114) | (23922/23941) | (99/118) | (23922/23937) |
| | Loss | 84.21 | 99.95 | 82.05 | 99.95 |
| | | (32/38) | (12796/12803) | (32/39) | (12796/12802) |
| | Overall | 86.18 | 99.93 | 83.44 | 99.94 |
| | | (131/152) | (36718/36744) | (131/157) | (36718/36739) |

TABLE 13
Concordance: Copy Number Alterations-Standard extraction process vs.
manual DNA extraction replicate A (non-equivocal, non-VUS CNAs)

| Variant Types | | PPA | NPA | PPV | NPV |
|---|---|---|---|---|---|
| CNA | Amplification | 89.90 | 99.93 | 84.76 | 99.96 |
| | | (89/99) | (23086/23102) | (89/105) | (23086/23096) |
| | Loss | 70.27 | 99.96 | 83.87 | 99.91 |
| | | (26/37) | (12349/12354) | (26/31) | (12349/12360) |
| | Overall | 84.56 | 99.94 | 84.56 | 99.94 |
| | | (115/136) | (35435/35456) | (115/136) | (35435/35456) |

TABLE 14
Concordance: Copy Number Alterations-Standard extraction process vs.
manual DNA extraction replicate B (non-equivocal, non-VUS CNAs)

| Variant Types | | PPA | NPA | PPV | NPV |
|---|---|---|---|---|---|
| CNA | Amplification | 91.67 | 99.98 | 95.19 | 99.96 |
| | | (99/108) | (23392/23397) | (99/104) | (23392/23401) |
| | Loss | 63.16 | 99.94 | 77.42 | 99.89 |
| | | (24/38) | (12490/12497) | (24/31) | (12490/12504) |
| | Overall | 84.25 | 99.97 | 91.11 | 99.94 |
| | | (123/146) | (35882/35894) | (123/135) | (35882/35905) |

For non-equivocal and non-VUS copy number amplification, PPA of standard process and warm lysate process compared to each manual DNA extraction replicate is higher than 88%, better than the PPA between two manual DNA extraction replicates. But the warm lysate extraction has lower PPV because of more non-equivocal and non-VUS copy number amplifications detected from the warm lysate process (>130) than the standard process (about 105) and manual DNA extraction (about 118). The standard process PPA of non-equivocal and non-VUS copy number loss is only 68.42% (26/38) compared to manual DNA extraction replicate A and 61.54% (24/39) manual DNA extraction process replicate B, while the PPA is over 80% between the two manual DNA extraction replicates and between warm lysate process and manual DNA extraction. A few more copy number losses were missed in the standard process but detected in manual DNA extraction and warm lysate process. Overall, combining copy number amplifications and loss, warm lysate had higher PPA than standard and two manual DNA extraction replicates, but standard process exhibited higher PPV than warm lysate and two manual DNA extraction replicates.

In conclusion, the warm lysate method (arm 4) had higher overall PPA than standard AutoLys method (arm 2), alleviated clogging for both DNA and RNA extraction, and resulted in an increased LC pass rate for RNA. The warm lysate method is an improvement to the standard AutoLys method as it alleviates clogging, while maintaining comparably high sequencing and variant detection performance to baseline RNA and baseline DNA methods. The automated warm lysate method can be used to extract RNA and DNA, from a single 40 micron FFPE curl, at comparable levels to manual DNA only extraction (DNA baseline method) and RNA only extraction (RNA baseline method) from two 40 micron FFPE curls (one 40 micron curl for DNA extraction and one 40 micron curl for RNA extraction).

### Example 5. Comparison of Standard and Mineral Oil Extraction Process

The standard AutoLys method, as discussed in the preceding section, was found to produce a wax layer post-centrifugation. The standard method was compared with the mineral oil extraction process described in Example 2, above, and as outlined in the middle column of FIG. 3A and middle column of FIG. 3B.

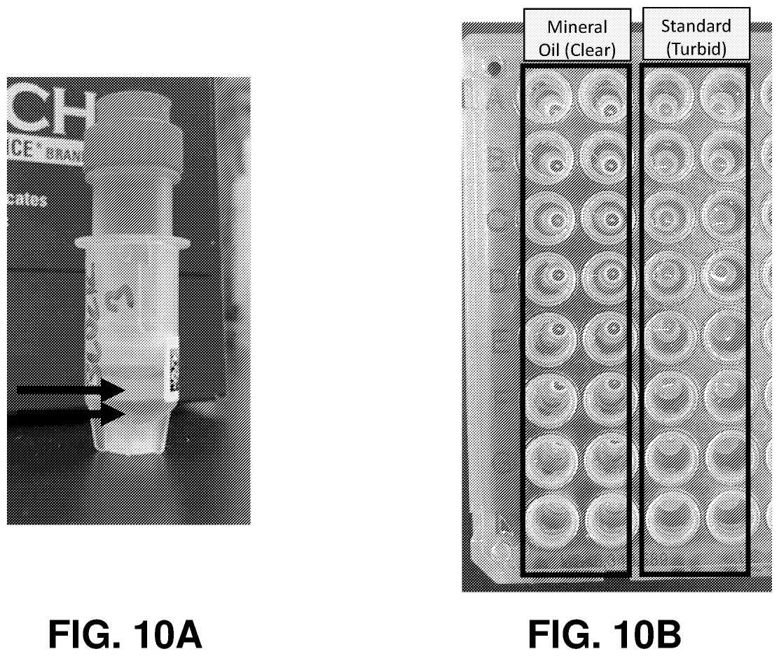

As shown in FIG. 10A, the mineral oil extraction process produced a paraffin layer that remains in the inner tube (indicated by the white arrow in FIG. 10A) and left a clear eluate with minimal residual paraffin in the lysate tube (see black arrow in FIG. 10A). As shown in the left two columns of FIG. 10A, each of the tested samples extracted with the mineral oil process had a clear eluate. In contrast, using the standard process (right two columns of FIG. 10B), a turbid eluate was produced, indicating the presence of paraffin in the eluate. The results show that the improved mineral oil extraction process can effectively separate the paraffin portion from the lysate portion, in contrast to the standard process (as shown in FIG. 2 and the right two columns of FIG. 10B), which leaves a wax (paraffin) layer post-centrifugation.

### Example 6. Low Tissue Volume Samples with Warm Lysate Method

This experiment was conducted in order to assess the feasibility of processing low tissue volume samples with the warm lysate co-extraction method described in Example 1 (see also right columns of FIG. 3A and FIG. 3B). Three different sample types were prepared, standard FFPE tissue curls (2×20 um curls for a total of 40 um), FFPE tissue slides (8×5 um slides for a total of 40 μm; 2 replicates), and low volume 14 gauge needle punches (1×1 mm depth) from 20 unique FFPE blocks representing 10 different tumor types (bladder, lung, skin, breast, ovary, kidney, prostate, pancreas, liver, & colon). All samples were extracted using the warm lysate co-extraction method and put through the standard library prep, hybrid capture, and sequencing processes. Table 15 below, shows the median tissue volume, lowest tissue volume, and maximum tissue volume for each input type.

TABLE 15
Tissue volumes

| | Median Tissue | Minimal Tissue | Maximal Tissue |
|---|---|---|---|
| Input Type | Volume (mm³) | volume (mm³) | volume (mm³) |
| Large Volume | 5.36 | 1.44 | 24 |
| Curls | | | |
| Large Volume | 5.36 | 1.44 | 24 |
| Slides | | | |
| Needle Punches | 1 | 1 | 1 |
| (14 ga) | | | |

TABLE 16
DNA Lab Metrics Summary

| | | Extraction | | | | | |
|---|---|---|---|---|---|---|---|
| | | per unit | | | | | |
| | Total | tissue vol | | LC | | HC | HC Pass |
| | Extraction | Median | DNAx | Median | LC Pass | Median | Rate |
| Input | Median | Yield | Pass Rate | Yield | Rate | Yield | (140 ng- |
| type | Yield (ng) | (ng/mm³) | (>55 ng) | (ng) | (>545.45 ng) | (ng) | 1782 ng) |
| Control | 9,397 | 1,146 | 100% | 10,901 | 100% | 297 | 100% |
| curl | | | | | | | |
| N = 20 | | | | | | | |
| Needle | 2,301 | 2,301 | 100% | 10,262 | 100% | 336 | 100% |
| Punch | | | | | | | |
| N = 20 | | | | | | | |
| Slide 1 | 8,327 | 1,324 | 100% | 10,054 | 100% | 274 | 100% |
| N = 20 | | | | | | | |
| Slide 2 | 11,117 | 1,395 | 100% | 11,686 | 100% | 281 | 100% |
| N = 20 | | | | | | | |

Table 16, above, shows a summary of the DNA Lab Metrics for extraction from different tissue inputs. Extraction per unit tissue volume is a measure of how many ng of DNA were extracted per mm³ of tissue. DNA extraction (DNAx) pass rate is a measure of how often >55 ng of DNA is collected. Library Construction (LC) pass rate is a measure of how often a DNA library with >545.45 ng DNA is generated. Hybrid Capture (HC) pass rate is a measure of how often a captured library with 140-1782 ng DNA for sequencing is obtained. Using the warm lysate process, there was a high success rate for DNAx, LC yield, and HC yield for all tissue input amounts. As expected, total DNA yield for the low tissue volume needle punches is lower than the 40 μm control curls and slides, however, the yield per unit of input tissue is comparable. The analyses demonstrates that the warm lysate process can be used to effectively extract nucleic acids from small amounts of tissue.

TABLE 17
DNA Sequencing Metrics Summary

| | | | | | | | Median |
|---|---|---|---|---|---|---|---|
| | | | | | | | Copy |
| | Mean | Pre | Median | Exon | | Medina | Number |
| | Insert | dup % | Exon | Coverage | | Error | Noise |
| Input type | Size | selected | Coverage | >250X | Median % Chimera | Rate | Score |
| Control | 142 | 71% | 1,054 | 99.8% | 0.79 | 0.28 | 5 |
| curl | | | | | | | |
| N = 20 | | | | | | | |
| Needle | 143 | 72% | 1,068 | 99.8% | 0.72 | 0.27 | 5.5 |
| Punch | | | | | | | |
| N = 20 | | | | | | | |
| Slide 1 | 147 | 72% | 1,039 | 99.6% | 0.74 | 0.28 | 4.5 |
| N = 20 | | | | | | | |
| Slide 2 | 145 | 71.5% | 1,049 | 99.7% | 0.84 | 0.27 | 5 |
| N = 20 | | | | | | | |

Table 17, above, shows a summary of the DNA sequencing metrics for each extraction method. Pre-dup % selected is the percent of reads mapping to the regions targeted by hybrid capture baits. Median insert size is the size of nucleic acid fragments between the adapters. % chimeras is the percentage of read chimeras. For median exon coverage, sequence coverage is number of times each base in the exon regions is covered in the sequence data; the average sequence coverage is calculated for each exon, and then the median of these values is used. Exon coverage pass rate was assessed at >250× coverage>500× coverage. CNA noise score is a score to measure the noise in cupy number plots. These analyses demonstrate that the sequencing metrics performance was comparable across all sample input types and that sample success rate for coverage (>250×) was greater than 99% for all samples. The warm lysate method can be used to effectively extract high quality nucleic acids from small amounts of tissue.

### Example 7. Additional Low Tissue Volume Samples with Warm Lysate Method

This experiment was conducted in order to assess the feasibility of processing low tissue volume samples with the warm lysate co-extraction method (described above including in Example 1 and right columns of FIG. 3A and FIG. 3B). Two different low volume sample types were prepared: 0.625 mm³ FFPE tissue volume slides (2 replicates) and 0.377 mm³ FFPE tissue volume curls (2 replicates) from 10 unique FFPE blocks representing 8 different tumor types (bladder, lung, breast, ovary, kidney, prostate, pancreas, and liver). All samples were extracted using the warm lysate co-extraction method and put through the standard library prep, hybrid capture, and sequencing processes. Table 18, below, shows the median tissue volume, lowest tissue volume, and maximum tissue volume for each input type.

TABLE 18
Tissue volumes.

| | Median Tissue | Minimal Tissue | Maximal Tissue |
|---|---|---|---|
| Input Type | Volume (mm\hat{ }3) | volume (mm\hat{ }3) | volume (mm\hat{ }3) |
| Small Volume | 0.625 | 0.625 | 0.625 |
| Slides | | | |
| Small Volume | 0.377 | 0.377 | 0.377 |
| Curls | | | |

TABLE 19
DNA Lab Metrics Summary

| | DNAx | Median | | LC Pass | | |
|---|---|---|---|---|---|---|
| | Pass rate | DNA | LC | rate | | HC Pass |
| Input type | >50 ng | Yield | Yield | >545 ng | HC | rate >140 |
| Curl | 100% | 1,045 | 8,200 | 100% | 307 | 100% |
| (0.377 mm3) | | | | | | |
| N = 20 | | | | | | |
| Slide | 100% | 506 | 7,248 | 100% | 309 | 100% |
| (0.625 mm3) | | | | | | |
| N = 20 | | | | | | |

Table 19, above, shows a summary of the DNA Lab Metrics for extraction from different tissue inputs. DNA extraction (DNAx) pass rate is a measure of how often >50 ng of DNA is collected. Library Construction (LC) pass rate is a measure of how often a DNA library with >545.ng DNA is generated. Hybrid Capture (HC) pass rate is a measure of how often a captured library with >140 ng DNA for sequencing is obtained. Using the warm lysate process, there was a high sample success rate for DNA extraction, LC and HC yields (100% pass rate). The analyses demonstrate that the warm lysate method can be used to effectively extract nucleic acids from small amounts of tissue.

TABLE 20
DNA Sequencing Metrics Summary

| | | | | | | | | Median |
|---|---|---|---|---|---|---|---|---|
| | | | | | | | | Copy |
| | Est | Median | Pre-dup | | | Median | Exon | Number |
| | Library | Insert | % | % | Error | Exon | Coverage | Noise |
| Input type | Size | size | selected | Chimera | rate | Coverage | ≥250X | Score |
| Curl | 3.35e+7 | 154 | 70% | 0.57 | 0.27 | 861 | 99.1% | 6 |
| (0.377 mm3) | | | | | | | | |
| Slide | 2.86e+7 | 151 | 71% | 0.45 | 0.27 | 778 | 99.2% | 7 |
| (0.625 mm3) | | | | | | | | |

Table 20, above, shows a summary of the DNA Sequencing Metrics for each extraction method. Estimated library size is the number of unique fragments in the library. Pre-dup % selected is the percent of reads mapping to the regions targeted by hybrid capture baits. Median insert size is the size of nucleic acid fragments between the adapters. % chimeras is the percentage of read chimeras. For median exon coverage, sequence coverage is the number of times each base in the exon regions is covered in the sequence data; the average sequence coverage is calculated in each exon, and then the median of these values is used. Exon coverage pass rate was assessed at >250× coverage and >500× coverage. CAN noise score is a score to measure the noise in copy number plots. These analyses demonstrate that high sequencing performance (coverage, insert size, % pre-dup selected, error rate, chimeras, CN noise) was observed for both 0.377 m³ curls and 0.625 mm³ slides. More than 99% samples had >250×. These results demonstrate that the warm lysate process can be used to effectively extract nucleic acids from small amounts of tissue.

1-277. (canceled)

278. A method of extracting RNA and/or DNA, wherein the method comprises:
a) providing a paraffin-embedded sample;
b) removing paraffin from the paraffin-embedded sample, thereby generating a deparaffinized sample, wherein the paraffin is removed by
i) inducing a phase transition in the paraffin and separating the paraffin from the sample or
ii) contacting the paraffin-embedded sample with an immiscible solvent and separating the paraffin from the sample; and
c) extracting RNA and/or DNA from the deparaffinized sample.

279. A method of detecting alterations in RNA and/or DNA comprising:
a) extracting RNA and/or DNA from the deparaffinized sample by the method of claim 278; and
b) analyzing the extracted RNA and/or DNA to detect alterations in the RNA and/or DNA.

280. A method of reducing the level of paraffin in a sample comprising RNA or DNA extracted from a paraffin embedded sample, wherein the method comprises
i) extracting RNA and/or DNA by the method of claim 278; and
ii) purifying the extracted RNA and/or DNA to provide a purified RNA and/or DNA sample.

281. A method of improving separation of paraffin from a paraffin embedded sample, wherein the method comprises extracting RNA and/or DNA by the method of claim 278.

282. A method of improving library construction for nucleic acid sequencing, wherein the method comprises
i) extracting RNA and/or DNA by the method of claim 278 to produced extracted RNA and/or DNA; and
ii) preparing a sequencing library from the extracted RNA and/or DNA for sequencing the RNA and/or the DNA.

283. The method of claim 279, wherein the alteration in the RNA and/or DNA is selected from the group consisting of:
a) a copy number alteration;
b) a point mutation;
c) an in-frame deletion of one or more codons;
d) an intragenic deletion;
e) an intragenic insertion;
f) a deletion of a full gene;
g) an inversion;
h) an interchromosomal translocation;
i) a tandem duplication;
j) a gene fusion;
k) a genomic rearrangement that comprises an intron sequence; and
l) a gene amplification or duplication.

284. The method of claim 279, wherein the alteration in the RNA and/or the DNA is a copy number alteration.

285. The method of claim 281, wherein the method is performed in a liquid handling robot comprising a filter.

286. The method of claim 285, wherein the filter is a spin filter.

287. The method of claim 278, wherein contacting the paraffin-embedded sample with an immiscible solvent and separating the paraffin from the sample does not comprise dissolving the paraffin with a miscible solvent.

288. The method of claim 278, wherein the phase transition is melting.

289. The method of claim 278, wherein inducing a phase transition in the paraffin and separating the paraffin from the sample comprises heating and centrifuging the paraffin-embedded sample.

290. The method of claim 289, wherein the paraffin-embedded sample is heated to about 50° C. to about 80° C.

291. The method of claim 289, wherein the paraffin-embedded sample is heated for about 5 minutes, about 10 minutes, about 15 minutes, about 20 minutes, about 25 minutes, about 30 minutes, or about 35 minutes.

292. The method of claim 278, wherein inducing a phase transition in the paraffin and separating the paraffin from the sample comprises centrifuging and filtering the paraffin-embedded sample.

293. The method of claim 292, wherein the paraffin-embedded sample is centrifuged at 1600 to 2000 rcf.

294. The method of claim 278, wherein the immiscible solvent is mineral oil.

295. The method of claim 278, wherein the paraffin-embedded sample is contacted with about 300, 350, 400, 450, 500, or 550 μL of mineral oil.

296. The method of claim 278, wherein contacting the paraffin-embedded sample with an immiscible solvent and separating the paraffin from the sample comprises incubating the paraffin-embedded sample in mineral oil at about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., or about 75° C.

297. The method of claim 294, wherein the mineral oil contacts the paraffin-embedded sample for about 5 minutes, about 10 minutes, about 15 minutes, about 20 minutes, about 25 minutes, about 30 minutes, or about 35 minutes.

298. The method of claim 294, wherein the mineral oil contacts the paraffin-embedded sample while shaking at about 25 rpm, about 50 rpm, about 100 rpm, about 200 rpm, about 300 rpm, about 400 rpm, about 500 rpm, about 600 rpm, about 700 rpm, about 800 rpm, about 900 rpm, about 1000 rpm, or about 1100 rpm.

299. The method of claim 278, wherein step b) of removing paraffin from the paraffin-embedded sample is automated.

300. The method of claim 278, wherein the method is automated.

301. The method of claim 278, wherein 12, 24, 48, or 96 paraffin-embedded samples are processed in parallel.

302. The method of claim 278, wherein the method is performed using a liquid handling robot.

303. The method of claim 278, wherein the fixed paraffin-embedded sample is selected from the group consisting of a formalin-fixed sample, an ethanol-fixed sample, and a methanol-fixed sample.

304. The method claim 278, wherein the paraffin-embedded sample is derived from a formalin-fixed paraffin-embedded (FFPE) tissue, from a cryopreserved tissue, or from a fresh-frozen tissue.

305. A method of extracting an analyte, wherein the method comprises:
a) providing an embedded sample comprising an analyte, wherein the sample is embedded in an embedding agent;
b) removing the embedding agent from the embedded sample, thereby generating a de-embedded sample, wherein the embedding agent is removed by inducing a phase transition in the embedding agent and separating the embedding agent from the sample;
i) inducing a phase transition in the embedding agent and separating the embedding agent from the sample, or
ii) contacting the embedded sample with an immiscible solvent and separating the embedding agent from the sample; and
c) extracting the analyte from the de-embedded sample.

306. A method of detecting an analyte, wherein the method comprises:
a) extracting the analyte by the method of claim 305; and
d) analyzing the extracted analyte to detect the analyte.

307. A method of reducing the level of embedding agent in an analyte sample extracted from an embedded sample, wherein the method comprises:
a) extracting an analyte by the method of claim 305; and
d) purifying the extracted analyte to provide a purified analyte sample.

308. A method of improving separation of an embedding agent from an embedded sample, wherein the method comprises extracting an analyte by the method of claim 305.

309. A method of extracting RNA and DNA from a paraffin-embedded sample, wherein the method comprises:
a) incubating the sample with a protease;
b) incubating the sample at 50° C. to 80° C. for 1 to 40 minutes;
c) centrifuging the sample at a high speed to separate the paraffin from a first lysate comprising the RNA;
d) aspirating the first lysate comprising the RNA;
e) isolating the RNA from the first lysate;
f) incubating the sample from step c) with a protease;
g) incubating the sample from step f) at 50° C. to 80° C. for 2-48 hours;
h) centrifuging the sample at high speed;
i) aspirating a second lysate comprising the DNA; and
j) isolating the DNA from the second lysate.

310. A method of extracting RNA and DNA from a paraffin-embedded sample, wherein the method comprises:
a) adding mineral oil to the paraffin-embedded sample;
b) melting the paraffin by incubating the sample at 50° C. to 80° C. for 1 to 40 minutes;
c) cooling the sample to room temperature; and
d) extracting the RNA and DNA according to the method of claim 309.
